(19) United States
(12) Patent Application Publication
(10) Pub. No.: US 2022/0154280 A1
(43) Pub. Date: May 19, 2022

(54) A DIAGNOSTIC AND PROGNOSTIC TEST FOR MULTIPLE CANCER TYPES BASED ON TRANSCRIPT PROFILING

(71) Applicants: Edward Victor PROCHOWNIK, Pittsburgh, PA (US); James Matthew DOLEZAL, Chicago, IL (US); UNIVERSITY OF PITTSBURGH-OF THE COMMONWEALTH SYSTEM OF HIGER EDUCATION, Pittsburgh, PA (US)

(72) Inventors: Edward Victor PROCHOWNIK, Pittsburgh, PA (US); James Matthew DOLEZAL, Chicago, IL (US)

(21) Appl. No.: 17/423,648

(22) PCT Filed: Jan. 17, 2020
(86) PCT No.: PCT/US2020/014011

§ 371 Date: Jul. 16, 2021

Related U.S. Application Data
(60) Provisional application No. 62/793,722, filed on Jan. 17, 2019.

(51) Int. Cl. C12Q 1/6886

(52) U.S. Cl. CPC C12Q 1/6886; C12Q 2600/158; C12Q 2600/118; C12Q 2600/106

(57) ABSTRACT

Disclosed herein t-SNE-assisted clustering revealed that the expression of certain cancer pathway transcripts are correlated with certain cancer types. In one aspect, disclosed herein are methods for diagnosis and prognosis of a cancer using cancer pathway transcript expression.

This application claims the benefit of U.S. Provisional Application No. 62/793,722, filed on Jan. 17, 2019, which is incorporated herein by reference in its entirety.

This invention was made with government support under Grant no. CA174713 awarded by the National Institutes of Health. The government has certain rights in the invention.

### I. BACKGROUND

Next-generation DNA and RNA sequencing have identified recurrent mutations, rearrangements and altered gene expression in many cancers. These changes are often associated with novel tumor subtypes, behaviors and prognoses not appreciated using traditional pathological assessments. An example of the clinical utility of such molecular testing is the MammaPrint assay, which relies on the differential expression of 70 transcripts in stage I and stage II breast cancer to identify those individuals most likely to benefit from adjuvant chemotherapy. Another example is THYROSEQ®, which utilizes a combination of DNA and transcript analyses to detect copy number variations, mutations, fusions and expression differences of 114 genes to classify thyroid tumors, particularly those of indeterminant histology. Despite their utility, these and other such tests focus only on specific cancer types or subtypes. As yet, no reliable method has proven to be of prognostic value across multiple cancers. What are needed are new diagnostic and prognostic methods that can be proven across multiple cancers.

### II. SUMMARY

Disclosed are methods related to making a diagnosis or prognosis of a cancer in a subject.

In one aspect, disclosed herein are methods for diagnosing, monitoring the progress of, and/or providing a prognosis of a cancer in a subject, said method comprising a) receiving RNA expression data for a sample of tumor; b) determining a global cancer pathway transcript (CPT) expression profile for the sample based on the RNA expression data for one or more cancer-related pathways; and c) providing a diagnosis, prognosis, or treatment recommendation based on the global CPT expression profile; wherein a change in one or more cancer pathway transcript relative to a control indicates an increase in survivability of the subject for the cancer.

Also disclosed are methods of for diagnosing, monitoring the progress of, and/or providing a prognosis of a cancer in a subject of any preceding aspect, wherein the one or more cancer-related pathways is selected from the group consisting of cell cycle pathway, Notch pathway, Purine biosynthesis pathway, TP53 pathway, Hippo pathway, TCA cycle pathway, Wnt pathway, PI3K pathway, Pyrimidine Biosynthesis pathway, TGF-β pathway, Myc pathway, and Pentose Phosphate Pathway (PPP).

In one aspect disclosed are methods of for diagnosing, monitoring the progress of, and/or providing a prognosis of a cancer in a subject of any preceding aspect, wherein the cancer is selected from the group consisting of Acute myeloid leukemia (AML), Adrenocortical carcinoma (ACC), Bladder urothelial carcinoma (BLCA), Brain lower grade Glioma (BLGG), Breast invasive carcinoma (BRIC), triple negative breast cancer (TNBC), luminal A breast cancer, cervical squamous cell carcinoma and endocervical adenocarcinoma (CESC), Cholangiocarcinoma (CHOL), Glioblastoma multiform (GBM), Head and neck squamous cell carcinoma (HNSC), High risk Wilms tumor (HRWT), Kidney chromophobe (KICH), Clear cell renal cancer (KIRC), Kidney renal papillary cell carcinoma (KURP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromacytoma/paraganglioneuroma (PCPG), Rectal adeno-carcinoma (READ), Sarcoma (SARC). Metastatic skin cutaneous melanoma (Metastatic SKCM), Stomach adenocarcinoma (STAD), Thymoma (THYM), Thyroid cancer (THYC), Uterine carcinosarcoma (UCSC), Uterine corpus endometrial carcinoma (UCEC), and Uveal melanoma (UVM).

Also disclosed are methods of for diagnosing, monitoring the progress of, and/or providing a prognosis of a cancer in a subject of any preceding aspect, further comprising receiving the sample of tumor, extracting RNA from the sample, isolating a plurality of CPTs from the extracted RNA, and obtaining the RNA expression data from the isolated CPTs.

Alternatively or additionally, in some implementations, the RNA expression data can include RNA-seq data. Alternatively or additionally, in some implementations, the RNA expression data can include microarray data.

In one aspect disclosed are methods of for diagnosing, monitoring the progress of, and/or providing a prognosis of a cancer in a subject of any preceding aspect, further comprising receiving respective RNA expression data and respective clinical information for each of a plurality of tumors from a database, determining respective global CPT expression profiles for the tumors in the database based on the respective RNA expression data, identifying recurring patterns of CPT expression among the tumors in the database, and comparing the recurring patterns of CPT expression with the respective clinical parameters.

Alternatively or additionally, in some implementations, the step of identifying recurring patterns of CPT expression among tumors in the database can include applying a machine learning model that analyzes linear and non-linear relationships among the respective relative expression for each of the plurality of CPTs. Optionally, the machine learning model can be t-distributed stochastic neighbor embedding (t-SNE).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows 3D t-SNE plots of transcript clusters from each of the twelve cancer-related pathways (Table 1). For each pathway, two representative tumor types are shown. Numbers at the bottom left of each profile indicate the perplexity value under which t-SNE clustering was performed and that was used to optimize visualization of the t-SNE clusters. FIGS. 2, 3, 4, 5, 6, 7, 8, 9, 10, 11, 12, and 13 show t-SNE profiles of additional relevant tumor types for each pathway. See Table 2 for the abbreviations used to describe each tumor group. See Table 3 for the specific parameters that were used to generate each t-SNE cluster.

FIG. 2 shows additional t-SNE profiles for select tumor types, excluding those shown in FIG. 1, demonstrating Cell Cycle Pathway transcript clustering.

FIG. 3 shows additional t-SNE profiles for select tumor types, excluding those shown in FIG. 1, demonstrating Wnt Pathway transcript clustering.

FIG. 4 shows additional t-SNE profiles for select tumor types, excluding those shown in FIG. 1, demonstrating Notch Pathway transcript clustering.

FIG. 5 shows additional t-SNE profiles for select tumor types, excluding those shown in FIG. 1, demonstrating PI3K Pathway transcript clustering.

FIG. 6 shows additional t-SNE profiles for select tumor types, excluding those shown in FIG. 1, demonstrating Purine Biosynthesis Pathway transcript clustering.

FIG. 7 shows additional t-SNE profiles for select tumor types, excluding those shown in FIG. 1, demonstrating Pyrimidine Biosynthesis Pathway transcript clustering.

FIG. 8 shows additional t-SNE profiles for select tumor types, excluding those shown in FIG. 1, demonstrating TP53 Pathway transcript clustering.

FIG. 9 shows additional t-SNE profiles for select tumor types, excluding those shown in FIG. 1, demonstrating TGF-β Pathway transcript clustering.

FIG. 10 shows additional t-SNE profiles for select tumor types, excluding those shown in FIG. 1, demonstrating Hippo Pathway transcript clustering.

FIG. 11 shows additional t-SNE profiles for select tumor types, excluding those shown in FIG. 1, demonstrating Myc Pathway transcript clustering.

FIG. 12 shows additional t-SNE profiles for select tumor types, excluding those shown in FIG. 1, demonstrating TCA Cycle transcript clustering.

FIG. 13 shows additional t-SNE profiles for select tumor types, excluding those shown in FIG. 1, demonstrating Pentose Phosphate Pathway transcript clustering.

FIG. 14 shows Kaplan-Meier survival curves of patients based on t-SNE clustering profiles shown in FIG. 1. The survival curves shown here are those of tumor groups shown in FIG. 1 and distinguished by their t-SNE profiles. The patient groups being compared are indicated by the same colors used to present the t-SNE clusters. P values between individual groups are indicated only when significant. See FIGS. 15, 16, 17, 18, 19, 20, 21, 22, 23, 24, 25, and 26 for other relevant survival curves that correspond to the t-SNE profiles depicted in FIGS. 2, 3, 4, 5, 6, 7, 8, 9, 10, 11, 12, and 13.

FIG. 15 shows additional Kaplan-Meier survival curves for patients with distinct groups of Cell Cycle Pathway t-SNE clusters, excluding those shown in FIG. 14.

FIG. 16 shows additional Kaplan-Meier survival curves for patients with distinct groups of Wnt Pathway t-SNE clusters, excluding those shown in FIG. 14.

FIG. 17 shows additional Kaplan-Meier survival curves for patients with distinct groups of Notch Pathway t-SNE clusters, excluding those shown in FIG. 14

FIG. 18 shows additional Kaplan-Meier survival curves for patients with distinct groups of PI3K Pathway t-SNE clusters, excluding those shown in FIG. 14

FIG. 19 shows additional Kaplan-Meier survival curves for patients with distinct groups of Purine Biosynthesis Pathway t-SNE clusters, excluding those shown in FIG. 14.

FIG. 20 shows additional Kaplan-Meier survival curves for patients with distinct groups of Pyrimidine Biosynthesis Pathway t-SNE clusters, excluding those shown in FIG. 14.

FIG. 21 shows additional Kaplan-Meier survival curves for patients with distinct groups of TP53 Pathway t-SNE clusters, excluding those shown in FIG. 14.

FIG. 22 shows additional Kaplan-Meier survival curves for patients with distinct groups of TGF-β Pathway t-SNE clusters, excluding those shown in FIG. 14.

FIG. 23 shows additional Kaplan-Meier survival curves for patients with distinct groups of Hippo Pathway t-SNE clusters, excluding those shown in FIG. 14.

FIG. 24 shows additional Kaplan-Meier survival curves for patients with distinct groups of Myc Pathway t-SNE clusters, excluding those shown in FIG. 14.

FIG. 25 shows additional Kaplan-Meier survival curves for patients with distinct groups of TCA Cycle Pathway t-SNE clusters, excluding those shown in FIG. 14.

FIG. 26 shows additional Kaplan-Meier survival curves for patients with distinct groups of Pentose Phosphate Pathway t-SNE clusters, excluding those shown in FIG. 14

FIG. 27 shows a Summary of Kaplan-Meier survival results for every tumor type. The results are summarized from FIGS. 14, 15, 16, 17, 18, 19, 20, 21, 22, 23, 24, 25, and 26. Colored boxes indicate those instances in which the overall survival varied between at least 2 t-SNE clusters. Grey boxes indicate cases where survival differences between individual t-SNE clusters groups were not significant (NS) or where only a single t-SNE cluster was obtained. The P values listed are those between the two most disparate sets of survival curves for each comparison.

FIGS. 28A, 28B, 28C, 28D, and 28E show additional predictive power of sequential t-SNE analyses. Panel A shows the survival of clear cell kidney cancer patients based on t-SNE clustering of Purine Biosynthesis Pathway transcripts taken from FIG. 19 in the Supplementary Appendix. Panels B-E show the survival of t-SNE Clusters 1˜4 patients from A, respectively, after a second t-SNE analysis using Notch Pathway transcripts (FIG. 14). See FIGS. 41, 42, and 43 for similar analyses using 3 additional tumor groups.

FIG. 29 shows additional Random Forest Classifiers showing the individual transcripts in the Cell Cycle Pathway that were most deterministic of t-SNE profiles for each of 16 tumor types, not including those shown in FIG. 28.

FIG. 30 shows additional Random Forest Classifiers showing the individual transcripts in the Wnt Pathway that were most deterministic of t-SNE profiles for each of 9 tumor types, not including those shown in FIG. 28.

FIG. 31 shows additional Random Forest Classifiers showing the individual transcripts in the Notch Pathway that were most deterministic of t-SNE profiles for each of 5 tumor types, not including those shown in FIG. 28.

FIG. 32 shows additional Random Forest Classifiers showing the individual transcripts in the PI3K Pathway that were most deterministic of t-SNE profiles for each of 6 tumor types, not including those shown in FIG. 28.

FIG. 33 shows additional Random Forest Classifiers showing the individual transcripts in the Purine Biosynthesis Pathway that were most deterministic of t-SNE profiles for each of 6 tumor types, not including those shown in FIG. 28.

FIG. 34 shows additional Random Forest Classifiers showing the individual transcripts in the Pyrimidine Biosynthesis Pathway that were most deterministic of t-SNE profiles for each of 5 tumor types, not including those shown in FIG. 28.

FIG. 35 shows additional Random Forest Classifiers showing the individual transcripts in the TP53 Pathway that were most deterministic of t-SNE profiles for each of 7 tumor types, not including those shown in FIG. 28.

FIG. 36 shows additional Random Forest Classifiers showing the individual transcripts in the TGF-β Pathway that were most deterministic of t-SNE profiles for each of 11 tumor types, not including those shown in FIG. 28.

FIG. 37 shows additional Random Forest Classifiers showing the individual transcripts in the Hippo Pathway that were most deterministic of t-SNE profiles for each of 13 tumor types, not including those shown in FIG. 28.

FIG. 38 shows additional Random Forest Classifiers showing the individual transcripts in the Myc Pathway that were most deterministic of t-SNE profiles for each of 6 tumor types, not including those shown in FIG. 28.

FIG. 39 shows additional Random Forest Classifiers showing the individual transcripts in the TCA Pathway that were most deterministic of t-SNE profiles for each of 6 tumor types, not including those shown in FIG. 28.

FIG. 40 shows additional Random Forest Classifiers showing the individual transcripts in the Pentose Phosphate Pathway that were most deterministic of t-SNE profiles for each of 5 tumor types, not including those shown in FIG. 28.

FIGS. 41A, 41B, 41C, and 41D show additional predictive power of sequential t-SNE analyses in sarcoma. FIG. 41A shows the survival curve from FIG. 14 of patients with sarcomas based on t-SNE clusters from the Purine Biosynthesis Pathway. FIG. 41B shows Cluster 1 patients from 41A were further analyzed based on whether they could be categorized as Cluster 1 or Cluster 2 when analyzed for TGF-β Pathway transcripts. FIG. 41C shows that Cluster 2 patients from 41A were similarly categorized as in 41B. FIG. 41D shows that Cluster 3 patients from 41A were similarly categorized as in 41B.

FIGS. 42A, 42B, 42C, 42D, and 42E show Additional predictive power of sequential t-SNE analyses in clear cell kidney cancer. FIG. 42A shows survival curves from FIG. 19 of patients based on t-SNE clusters of transcripts from the Purine Biosynthesis Pathway. FIGS. 42B, 42C, 42D, and 42E show t-SNE Clusters 1˜4 patients, respectively, from 42A who were further stratified based on their t-SNE expression profiles of PI3K Pathway t-SNE Clusters 1-3 (FIG. 18).

FIGS. 43A, 43B, 43C, 43D, and 43E show additional predictive power of sequential t-SNE analyses in head and neck squamous cell cancer. FIG. 43A shows the survival curve from FIG. 14 of patients based on t-SNE clusters of transcripts from the Myc Pathway. FIG. 43B shows that Cluster 1 patients from 43A were further analyzed based on whether they could be categorized as Cluster 1, Cluster 2, or Cluster 3 when analyzed for cell cycle pathway transcripts (43C, 43D, and 43E). Clusters 2-4 patients from 43A were similarly categorized as in 43B.

FIGS. 44A, 44B, 44C and 44D show whole transcriptome analysis further refines the predictive power of t-SNE profiling. FIG. 44A shows unsupervised hierarchical clustering of whole transcriptome profiles from 177 pancreatic adenocarcinomas. Three major groups were identified and are indicated by name (Dendro 1, Dendro 2, and Dendro 3) and by the green, blue and red horizontal bars, respectively, above the heat map. Within each Dendro group, individual tumors, previously classified by t-SNE for their expression patterns of purine biosynthesis family transcripts (Clusters 1-3) (FIG. 14) are indicated by the red, blue and yellow-colored bars, respectively, at the bottom of the heat map. FIG. 44B shows Kaplan-Meier survival curves of patients from each of the Dendro groups in A. FIG. 44C shows tumors from Purine Biosynthesis Pathway t-SNE Cluster 3 (unfavorable survival: FIGS. 1 and 14) were further divided according to the dendrogram group with which they associated and Kaplan-Meier curves were again generated. FIG. 44D shows similar to 44C, patients from Purine Biosynthesis Pathway t-SNE Cluster 1 (favorable survival) were also grouped according to the Dendro group with which they associated.

FIGS. 45A, 45B, 45C and 45D show whole transcriptome analysis refines the predictive power of Pyrimidine Pathway t-SNE profiling in renal clear cell carcinoma (KIRC). FIG. 45A shows hierarchical clustering of all KIRCs based on whole transcriptome profiling. Each tumor's t-SNE cluster is indicated and is derived from FIG. 14. FIG. 45B shows Kaplan-Meier survival curves of each of the Dendro groups from 45A. FIG. 45C shows all t-SNE Cluster 1 tumors with favorable survival (FIG. 14) were further categorized based on their Dendro Groupings. It can be seen that these tumors were associated with a worse overall survival if they fell into the Dendro 1 group. Similarly, FIG. 45D shows t-SNE cluster 2 tumors with overall unfavorable survival could be further sub-classified according to their Dendro group.

FIGS. 46A, 46B, 46C, and 46D show whole transcriptome analysis refines the predictive power of Myc Pathway t-SNE profiling in sarcoma (SARC). FIG. 46A shows Hierarchical clustering of all sarcoma patients identified 4 distinct Dendro Groups (1-4). The two t-SNE Clusters into which these tumors fell are indicated at the bottom of the heat map. Note that the Dendro 1 Group is particularly weighted with t-SNE Cluster 2 tumors having favorable survival. To a somewhat lesser extent, the Dendro 4 Group was more heavily populated by t-SNE Cluster 1 tumors with unfavorable survival. FIG. 46B shows the survival for each of the Dendro Groups in (46A) showing that Dendro Groups 1 and 2 were associated with relatively favorable survival whereas Dendro group 4 was associated with unfavorable survival. FIG. 46C shows that t-SNE Cluster 1 unfavorable survival tumors could be further subdivided based on their Dendro Group identities. FIG. 46D shows that t-SNE Cluster 2 favorable survival tumors could also be subdivided further based on there whole transcriptome profiles.

FIGS. 47A, 47B, 47C, 47D, and 47E show whole transcriptome analysis refines the predictive power of TCA Cycle Pathway in bladder urothelial cancer (BLCA). FIG. 47A shows hierarchical clustering of all tumors identified 4 Dendro Groups. Note that Dendro Groups 1 and 2 are over-represented by t-SNE Cluster 2 TCA Pathway tumors with an intermediate survival whereas Dendro Group 4 is over-represented by t-SNE Cluster 3 tumors with a relatively favorable survival (FIGS. 12 and 25). FIG. 47B shows Kaplan-Meier survival curves of each of the 4 Dendro Groups in (47A). FIGS. 47C, 47D, and 47E show Kaplan-Meier survival curves of each of the 3 t-SNE Groups. Note that the t-SNE Cluster 1 could not be further subdivided by further hierarchical clustering whereas both t-SNE Clusters 2 and 3 could.

FIGS. 48A, 48B, 48C, 48D, 48E, 48F, 48G, 48H, 48I, and 48J show t-SNE profiling can further refine survival prediction in specific breast cancer subtypes. FIG. 48A shows Kaplan-Meier survival of patients with TNBC and Luminal A tumors. Patients and survival information were compiled from TCGA. FIG. 48B shows t-SNE clusters of only TNBC and Luminal A tumors from (48A) using Wnt Pathway transcripts. These were derived from FIG. 3. FIG. 48C shows Kaplan-Meier survival of each of the t-SNE groups from (48B). NS=not significant. FIG. 48D shows t-SNE profiling of TNBC and Luminal A tumors using Myc Pathway transcripts. FIG. 48E shows Kaplan-Meier survival of each of the t-SNE groups from (48D). FIG. 48F shows random Forest classification of transcripts from the Wnt Pathway that were the most deterministic of survival for all TNBC patients from (48A). FIG. 48G shows expression levels of Sfrp2 transcripts in each of the t-SNE clusters of TNBCs from (48B). FIG. 48H shows random Forest classification of transcripts from the Myc Pathway that were the most deterministic of survival for all Luminal A patients from (A48). FIG. 48I shows expression levels of Myc transcripts in each of the t-SNE clusters of Luminal A tumors from (48D). FIG. 48J shows expression levels of Mxd2 transcripts in each of the t-SNE clusters of Luminal A tumors from (48D).

FIGS. 49A, 48B, 49C, 49D, and 49E show t-SNE profiling better predicts survival in tumors from individuals with advanced stage disease. FIG. 49A shows original t-SNE clusters of all primary bladder cancers profiled with TCA Cycle transcripts (from FIG. 12). FIG. 49B shows the t-SNE clusters from (49A) showing only Stage IV primary tumors (total=135). FIG. 49C shows differential survival of Stage IV patients from (49B). FIG. 49D shows t-SNE clustering of Stage IV only head and neck squamous cell cancers using Myc Pathway transcripts. See FIG. 1 for t-SNE clustering with all tumors. FIG. 49E shows the survival of patients from (49D) according to t-SNE cluster

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Genetic testing of cancers has improved diagnosis, risk-stratification and therapeutic decisions but has been difficult to extend beyond individual cancer types. Prior to the present disclosure, tests with broader predictive capabilities were lacking.

It is understood and herein contemplated that ribosomal proteins (RPs) participate in a variety of extra-ribosomal functions. In normal contexts, ribosome assembly from rRNAs and RPs is a tightly regulated process, with unassembled RPs undergoing rapid degradation. Disruption of ribosomal biogenesis by any number of extracellular or intracellular stimuli induces ribosomal stress, leading to an accumulation of unincorporated RPs. These free RPs are then capable of participating in a variety of extra-ribosomal functions, including the regulation of cell cycle progression, immune signaling, and cellular development. Many free RPs bind to and inhibit MDM2, a potentially oncogenic E3 ubiquitin ligase that interacts with p53 and promotes its degradation. The resulting stabilization of p53 triggers cellular senescence or apoptosis in response to the inciting ribosomal stress.

Given their role in regulating gene translation, cellular differentiation, and organismal development, it is perhaps unsurprising that altered RP expression has been implicated in human pathology. Indeed, an entire class of diseases referred to as “ribosomopathies,” has been shown to be associated with haploinsufficient expression or mutation in individual RPs. Ribosomopathy-like properties have also been observed in various cancers. It has recently been shown that RP transcripts (RPTs) were dysregulated in two murine models of hepatoblastoma and hepatocellular carcinoma in a tumor specific manner and in patterns unrelated to tumor growth rates. These murine tumors also displayed abnormal rRNA processing and increased binding of free RPs to MDM2, reminiscent of the aforementioned inherited ribosomopathies.

As described above, ribosomes, the organelles responsible for the translation of mRNA, are comprised of rRNA and approximately 80 RPs. Although canonically assumed to be maintained in equivalent proportions, some RPs have been shown to possess differential expression across tissue types. Dysregulation of RP expression occurs in a variety of human diseases, notably in many cancers, and altered expression of some RPs correlates with different tumor phenotypes and patient survival. Using RNAseq data from 10,423 patients in The Cancer Genome Atlas (TCGA), protein-coding transcripts were evaluated from 12 cancer-related signaling pathways in 34 cancer types. Rather than relying on absolute transcript levels, t-distributed stochastic neighbor embedding (t-SNE) was employed to identify expression patterns differences among each pathway's component transcripts. A machine learning-based dimensionality reduction technique for describing non-linear relationships among points in a data set, t-SNE was described in PCT Application No. PCT/US2018/42455, filed on Jun. 17, 2018 which is incorporated herein by reference in its entirety. The method described therein predicted survival in some cancers based on expression patterns of cancer pathway transcript.

t-SNE-assisted transcript pattern profiling with 212 genes from 12 cancer-related pathways allowed patient cohorts with significant long-term survival differences to be identified in 29 of 34 cancer types comprising 9097 individuals (87.3% of all cases). A curated 32 member transcript subset from each family that most commonly determined t-SNE profiles predicted survival in 16 cancer types (54.8% of all cases). When used in conjunction with transcripts from at least one other pathway, the predictive value of the subset increased to 30 of 34 cancer types, representing 91.8% of all cancers.

In one aspect, disclosed herein are methods for diagnosing, monitoring the progress of, and/or providing a prognosis of a cancer in a subject, said method comprising a) receiving RNA expression data for a sample of tumor; b) determining a global cancer pathway transcript (CPT) expression profile for the sample based on the RNA expression data for one or more cancer-related pathways; and c) providing a diagnosis, prognosis, or treatment recommendation based on the global CPT expression profile; wherein a change in one or more cancer pathway transcript relative to a control indicates an increase in survivability of the subject for the cancer.

It is understood and herein contemplated that transcript patterns in cancer-related pathways might be de-regulated in ways that recall CPTs and that also correlate with survival. t-SNE was used to apportion twelve cancer-related pathways, comprising 212 protein-coding transcripts into distinct expression pattern-related clusters, which were then compared for long-term survival. Accordingly, disclosed are methods of for diagnosing, monitoring the progress of, and/or providing a prognosis of a cancer in a subject, wherein the one or more cancer-related pathways is selected from the group consisting of cell cycle pathway, Notch pathway, Purine biosynthesis pathway, TP53 pathway, Hippo pathway, TCA cycle pathway, Wnt pathway, PI3K pathway, Pyrimidine Biosynthesis pathway, TGF-β pathway, Myc pathway, and Pentose Phosphate Pathway (PPP). It is understood and herein contemplated that for each pathway, there can be one or more CPTs that correlate with survival in a cancer. Accordingly, in one aspect, it is understood and herein contemplated that the CPTs measured in the cell cycle pathway comprises one or more of CDKN1A, CCND2, CDKN1B, CCND1, CDK4, CCND3, CDKN2C, CCNE1, CDK5, E2F3, CDK2, CDKN2A, RB1, E2F1, and/or CDKN2B; for the Notch pathway the CPTs comprise one or more of NOV, DNER, HDAC1, HES1, HES2, HES3, HES4, HES5, HEY1, CREBBP, CNTN6, NOTCH2, NOTCH1, NCOR1, FBXW7, HEYL, NOTCH4, NCOR2, NES2, NOTCH3, PSEN2, KDM5A, EP300, KAT2B, SPEN, JAG2, HEY2, THBS2, CUL1, MAML3, and/or ARRDC1; for the Purine biosynthesis pathway the CPTs comprise one or more of PPAT, GART, PFAS, PAICS, ADSL, ATIC, ADSSL1, ADSS, AK1, AK2, AK3, AK4, AK5, AK7, GMPS, GUK1, RRM1, RRM2, NME1, NME2, NME3, NME4, NME5, NME6, and/or NME7; for the TP53 pathway the CPTs comprise one or more of TP53, CHEK2, MDM4, RPS6KA3, MDM2, and/or ATM; for the Hippo pathway the CPTs comprise one or more of YAP1, WWTR1, TEAD2, STK4, STK3, SAV1, LATS1, LATS2, MOB1A, MOB1B, PTPN14, NF2, WWC1, TAOK1, TAOK2, TAOK3, CRB1, CRB2, CRB3, FAT1, FAT2, FAT3, FAT4, DCHS1, DCHS2, CSNK1E, and/or CSNK1D; for the TCA cycle pathway the CPTs comprise one or more of CS, IDH1, IDH2, SDHD, OGDH, IDH3A, SUCLA2, IDH3B, SDHA, OGDHL, SUCLG1, FH, ACO2, SUCLG2, MDH1, SDHB, ACO1, MDH1B, IDH3G, MDH2, and/or SDHC; for the Wnt pathway the CPTs comprise one or more of ZNFR3, WIF1, TLE1, TLE2, TLE3, TLE4, TCF7L1, TCF7L2, SFRP1, SFRP2, SFRP4, SFRP5, RNF43, LRP5, GSK3B, DKK4, DKK3, DKK2, DKK1, CTNNB1, AXIN1, AXIN2, APC, and/or AMER1, for the PI3K pathway the CPTs comprise one or more of PTEN, PIK3CB, AKT3, PPP2R1A, PIK3R1, RICTOR, RHEB, TSC2, PIK3CA, MTOR, AKT2, STK11, AKT1, TSC1, RPTOR, PIK3R2, INPP4B, and/or PIK3R3; for the Pyrimidine Biosynthesis pathway the CPTs comprise one or more of NME4, NME3, RRM1, CMPK1, NME5, CAD, DUT, ENPP3, CMPK2, NTPCR, RRM2, CTPS1, NME6, NME2, DHODH, ITPA, TYMS, NME7, NME1, UMPS, DTYMK, ENPP1, and/or CPTS2, TGF-β pathway the CPTs comprise one or more of TGFBR2, TGFBR1, ACVR1B, ACVR2A, SMAD2, SMAD3, and/or SMAD4; for the Myc pathway the CPTs comprise one or more of MXD4, MLXIPL, MAX, MXI1, MYC, N-MYC, MXD1, MXD2, MXD3, MLX, MNT, MYCL, MLXIP, MYCN, and/or MGA; and for the Pentose Phosphate Pathway (PPP) the CPTs comprise one or more of PGD, H6PD, TALDO1, PGLS, TKT, RPIA, RPE, G6PD, TKTL1, TKTL2, and/or RPEL1.

It is understood and herein contemplated that while a singular pathway such as the cell cycle pathway can be predictive of a large percentage of cancers, it can be desirable to perform expression analysis of multiple pathways to provide a more complete predictive analysis of cancers across many cancer types. For example, an CPT expression profile can be generated for the cell cycle pathway, the Wnt pathway, and the combined pathways. Accordingly, disclosed herein are methods of for diagnosing, monitoring the progress of, and/or providing a prognosis of a cancer in a subject, wherein the one or more cancer-related pathways is, one, two, three, four, five, six, seven, eight, nine, ten, eleven, twelve, or all thirteen of the cancer related pathways selected from the group consisting of cell cycle pathway, Notch pathway, Purine biosynthesis pathway, TP53 pathway, Hippo pathway, TCA cycle pathway, Wnt pathway, PI3K pathway, Pyrimidine Biosynthesis pathway, TGF-β pathway, Myc pathway, and Pentose Phosphate Pathway (PPP).

In one aspect, a database of RNA expression data that includes expression of CPTs (e.g., RNA-seq, whole transcriptome sequence data, or microarray data) for a plurality of tumors is received or accessed. Optionally, clinical data for the patients from which these tumors derive can also be received or accessed. Such a database can include, but is not limited to, The Cancer Genome Atlas (TCGA). RNA expression data that includes the expression of CPTs for a sample of tumor (sometimes referred to herein as “individual tumor sample”) is also obtained. The tissue of origin of this tumor may be known or unknown (e.g., an undifferentiated tumor). For example, a tissue sample from a tumor in a subject's organ (e.g., liver) is taken by a surgeon. The tissue sample can be taken, for example, by performing a biopsy. An examination of the cells in this sample by a pathologist may not reveal in which of the subject's tissues or organs (e.g., lungs, kidneys, stomach, liver, brain, skin, testicle, thymus, thyroid, colon, pancreas, ovary, etc.) the cancer arises because the cells may appear immature and/or primitive and therefore difficult to identify. It should be understood that the tissue of origin is relevant to diagnosis, prognosis, and/or treatment. For example, not only are ovarian colo-rectal and pancreatic cancers treated very differently but they have vastly different survival.

In some implementations, the RNA expression data for the individual tumor sample is received, for example, at a computing device. In other implementations, the sample of tumor is optionally received, for example, at a laboratory or other facility for analysis. In this case, the method can include extracting RNA from the sample and isolating CPTs from the same. After isolating the CPTs, the RP RNA expression data can be obtained by sequencing the same. This disclosure contemplates providing a kit for facilitating extraction of RNA from the sample and isolation of the CPTs. Techniques for extracting RNA, isolating RNAs, and sequencing are known in the art. Additionally, techniques for specifically isolating CPTs are similar to techniques that have been used for other transcripts. For example, in some implementations, magnetic beads with oligonucleotides corresponding to the compliment of the coding sequence of the CPTs can be used to isolate the CPTs. It should be understood that this is only one example technique for isolating the CPTs and that other techniques can be used with the bioinformatics methods described herein. Additionally, this disclosure contemplates obtaining RNA expression data using other techniques including, but not limited to, using microarray- or hybridization-βased systems. For example, it should be understood that the cancer pathway transcript (CPT) expression pattern for a sample can be determined using a DNA microarray. DNA microarrays are known in the art and are therefore not described in further detail herein. Accordingly, the RNA expression data can be of any type and in some embodiments comprises whole or partial transcriptome sequence data (e.g., RNA-seq), RP sequence data, and/or microarray hybridization data.

As shown herein, global cancer pathway transcript (CPT) expression patterns or profiles for tumors in the database are determined based on the RNA expression data for the tumors obtained and a global CPT expression profile can be generated based on the RNA expression data received for the individual tumor sample. 77. This disclosure contemplates that the global CPT expression patterns or profiles can be determined using a computing device. This can include a pre-processing step of calculating a respective relative expression for each of a plurality of CPTs. Pre-processing is performed on the raw RNA expression data received for the database of tumors and for the individual tumor sample. As described herein, expression profiling of 212 genes from 12 cancer-related profiles were generated using a machine learning model is used to identify patterns of CPT relative expression in the database of tumors while analyzing linear and non-linear relationships among the respective relative expression for each of the plurality of CPTs. As described herein, the machine learning model can optionally be t-distributed stochastic neighbor embedding (t-SNE). t-SNE has advantages as compared to data analysis techniques such as PCA, particularly because t-SNE is able to identify common patterns and features in a data set while accounting for both linear and non-linear relationships. Patterns of CPT expression that significantly associate with clinical parameters have been identified. The global CPT expression profile from the individual tumor sample can be compared to the aforementioned CPT expression patterns identified in the database. Optionally, as described herein, global CPT expression for the tumors in the database, as well the individual tumor sample, can be graphically displayed with clusters using a three-dimensional (3D) map. It should be understood that this allows the user to visualize patterns in the data set.

A tissue of origin, diagnosis, prognosis, or treatment recommendation is provided based on the comparison between the global CPT expression profile of the individual tumor sample and the CPT expression patterns (including individual genes and pathways) identified in the database. For example, at least one of a clinical parameter (e.g., survivability metric), a molecular marker, or a tumor phenotype can be provided. As described herein, in some implementations, the tissue of origin for the sample can be sub-classified based on the global CPT expression pattern for the sample. The sub-classification can then be used when providing the diagnosis, prognosis, or treatment recommendation. This disclosure contemplates that any of the aforementioned information can be provided using a computing device. The comparison between the individual patient sample and the database of tumors is performed with the use of a classifier model.

The disclosed methods can be used to diagnose, monitor the progress of, or provide a prognosis for any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed methods can be used to diagnose or provide a prognosis for is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer (including, luminal A and triple negative breast cancer (TNBC)), and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, pancreatic cancer, Acute myeloid leukemia (AML), Adrenocortical carcinoma (ACC), Bladder urothelial carcinoma (BLCA), Brain lower grade Glioma (BLGG), Breast invasive carcinoma (BRIC), cervical squamous cell carcinoma and endocervical adenocarcinoma (CESC), Cholangiocarcinoma (CHOL), Glioblastoma multiform (GBM), Head and neck squamous cell carcinoma (HNSC), High risk Wilms tumor (HRWT), Kidney chromophobe (KICH), Clear cell renal cancer (KIRC), Kidney renal papillary cell carcinoma (KURP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromacytoma/paraganglioneuroma (PCPG), Rectal adeno-carcinoma (READ), Sarcoma (SARC), Metastatic skin cutaneous melanoma (Metastatic SKCM), Stomach adenocarcinoma (STAD), Thymoma (THYM), Thyroid cancer (THYC), Uterine carcinosarcoma (UCSC), Uterine corpus endometrial carcinoma (UCEC), and Uveal melanoma (UVM). In one aspect, the cancer is not colon adenocarcinoma (COAD), esophageal cancer (ESOP), diffuse large B-cell lymphoma (DLBC), prostate cancer (PRAD), or testicular germ cell tumor (TGCT).

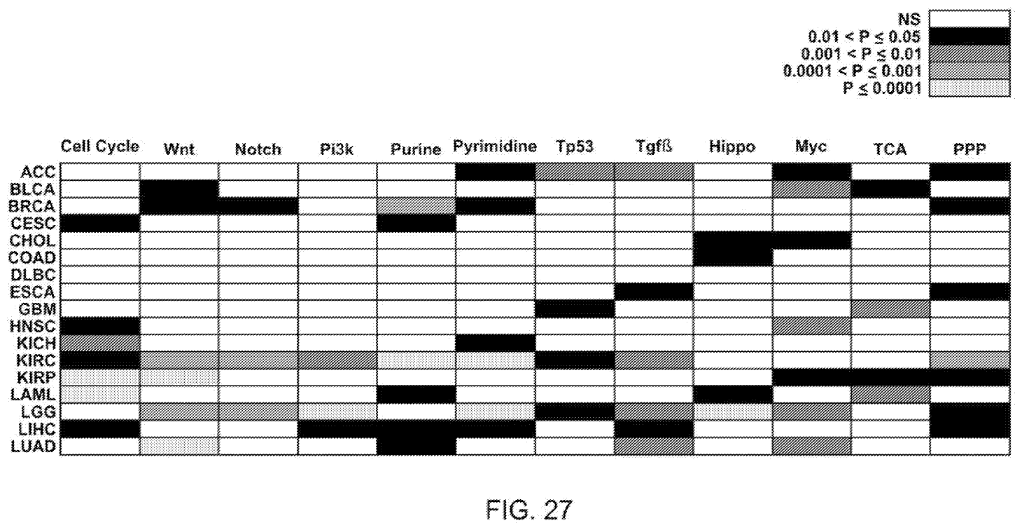

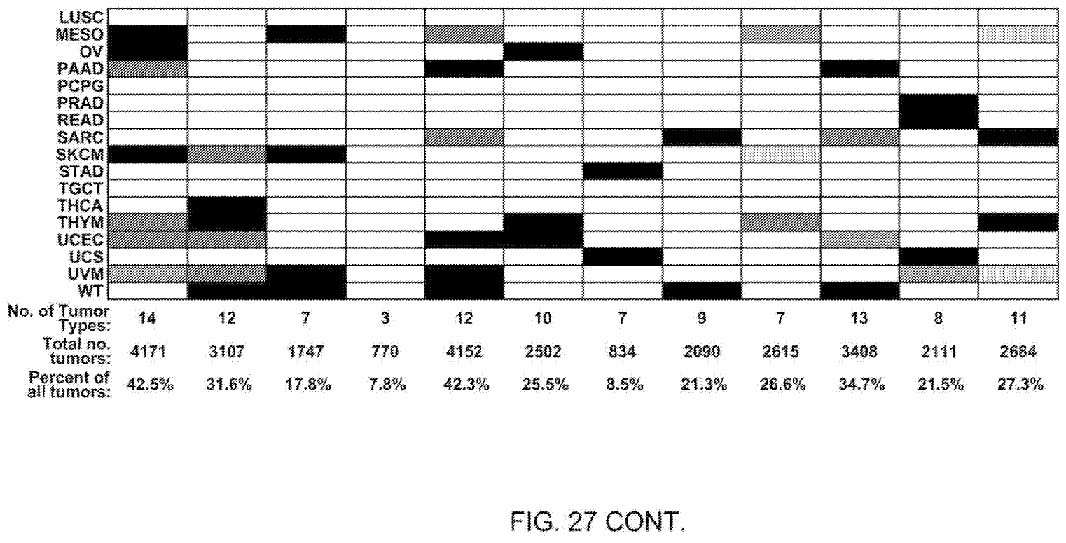

2. As shown in FIG. 27, for a given cancer, certain pathways are highly predictive survivability of a cancer. For example, for wherein the cancer comprises AML and the cancer related pathways comprise one or more of cell cycle, PI3K, Hippo, Purine Biosynthesis, and TCA; wherein the cancer comprises ACC and the cancer related pathways comprise one or more of cell cycle, TP53, TGF-β, Notch, Myc, Pyrimidine Biosynthesis, and TCA; wherein the cancer comprises BLCA and the cancer related pathways comprise one or more of TGF-β, Notch, Myc, Purine Biosynthesis, and TCA; wherein the cancer comprises BLGG and the cancer related pathways comprise one or more of cell cycle, TP53, TGF-β, PI3K, Hippo, Myc, Purine biosynthesis, and PPP; wherein the cancer comprises BRIC and the cancer related pathways comprise one or more of cell cycle, TP53, Myc, Purine Biosynthesis, and Pyrimidine Biosynthesis; wherein the cancer comprises CESC and the cancer related pathways comprise one or more of cell cycle, Myc, and Purine Biosynthesis; wherein the cancer comprises CHOL and the cancer related pathways comprise one or more of Notch and Myc; wherein the cancer comprises GBM and the cancer related pathways comprises TP53; wherein the cancer comprises HNSC and the cancer related pathways comprise one or more of cell cycle, and Myc; wherein the cancer comprises HRWT and the cancer related pathways comprise one or more of Wnt, TGF-β, Notch, PI3K, and Myc; wherein the cancer comprises KICH and the cancer related pathways comprise one or more of cell cycle, Wnt, PI3K, Purine Biosynthesis, and Pyrimidine Biosynthesis; wherein the cancer comprises KIRC and the cancer related pathways comprise one or more of cell cycle, Wnt, TP53, TGF-β, Hippo, Myc, Purine Biosynthesis, and TCA; wherein the cancer comprises KURP and the cancer related pathways comprise one or more of cell cycle, PI3K, Hippo, Purine Biosynthesis, Pyrimidine Biosynthesis, TCA, and PPP; wherein the cancer comprises LIHC and the cancer related pathways comprise one or more of Wnt, Purine Biosynthesis, TCA, and PPP; wherein the cancer comprises LUAD and the cancer related pathways comprise one or more of Wnt, PI3K, and Myc; wherein the cancer comprises LUSC and the cancer related pathways comprise one or more of cell cycle, Wnt, Hippo, and Purine Biosynthesis; wherein the cancer comprises MESO and the cancer related pathways comprise one or more of cell cycle, TGF-β, Notch, PI3K, Hippo, Purine Biosynthesis, Pyrimidine biosynthesis, and PPP; wherein the cancer comprises OV and the cancer related pathways comprises cell cycle; wherein the cancer comprises PAAD and the cancer related pathways comprise one or more of cell cycle, Myc, and Purine Biosynthesis; wherein the cancer comprises PCPG and the cancer related pathways comprises Wnt; wherein the cancer comprises READ and the cancer related pathways comprises cell cycle; wherein the cancer comprises SARC and the cancer related pathways comprise one or more of TGF-β, Myc, Purine Biosynthesis, Pyrimidine biosynthesis, and PPP; wherein the cancer comprises metastatic SKCM and the cancer related pathways comprise one or more of Wnt, Notch, and Hippo; wherein the cancer comprises STAD and the cancer related pathways comprise one or more of TGF-β and Hippo; wherein the cancer comprises THYM and the cancer related pathways comprise one or more of cell cycle, Wnt, TP53, Hippo, Purine Biosynthesis, Pyrimidine biosynthesis, and PPP; wherein the cancer comprises THYC and the cancer related pathways comprise one or more of cell cycle, PI3K, and TCA; wherein the cancer comprises UCSC and the cancer related pathways comprises TP53; and wherein the cancer comprises UCEC and the cancer related pathways comprise one or more of cell cycle, Wnt, Notch, Purine Biosynthesis, and Pyrimidine biosynthesis.

### A. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Prediction of Long-Term Survival in Cancer Patients Based on Expression Patterns of 212 or Fewer Protein-Coding Transcripts

The abundance of transcripts encoding the 80 ribosomal subunits vary by >300-fold in normal tissues and cancers. Using a machine learning technique known as t-distributed stochastic neighbor embedding (t-SNE), it was demonstrated that the expression patterns of these transcripts differ among normal tissues and cancers in distinct and reproducible ways that are unrelated to their absolute levels of expression. t-SNE profiling allows normal tissue and cancer types to be distinguished from one another. In many seemingly identical cancers, t-SNE revealed patient cohorts with multiple ribosomal protein transcript (RPT) patterns that in nine tumor types correlated with differences in survival.8

Ribosomal biogenesis is only one of numerous growth-related pathways that are de-regulated in cancer. To investigate whether transcript patterns in other pathways might also be de-regulated in ways that recall RPTs and that also correlate with survival, the transcriptomic data base of 10,423 tumors from The Cancer Genome Atlas was queried. t-SNE was used to apportion twelve cancer-related pathways, comprising 212 protein-coding transcripts into distinct expression pattern-related clusters, which were then compared for long-term survival. Finally, a curated list of 32 transcripts derived from the most predictive transcripts for each pathway was used to further refine the prognostic value of t-SNE profiling and reduce testing complexity.

a) Methods

(1) Selection of Transcripts

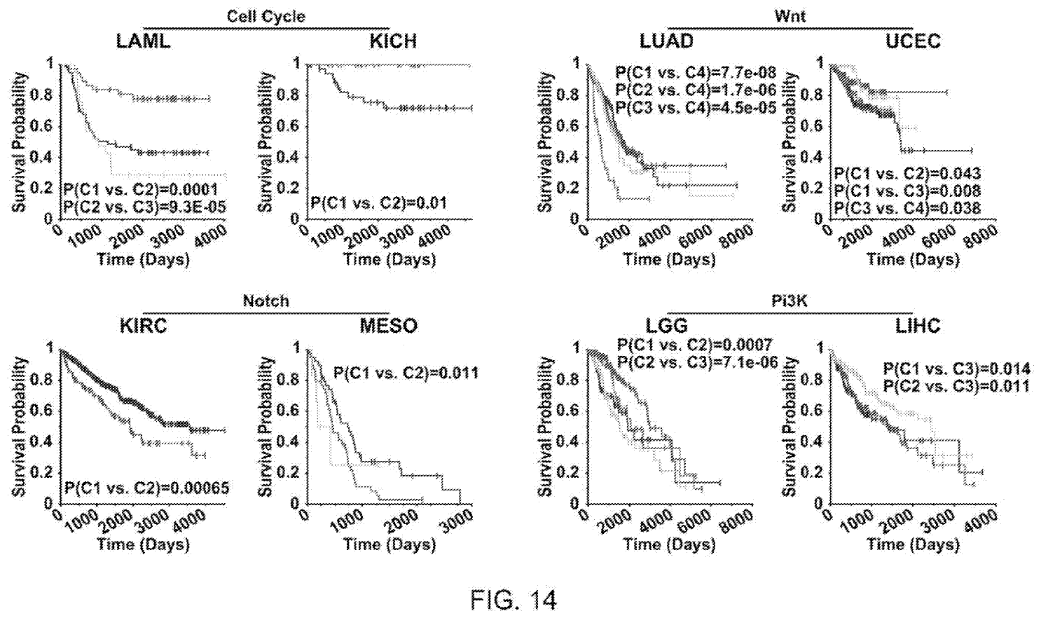

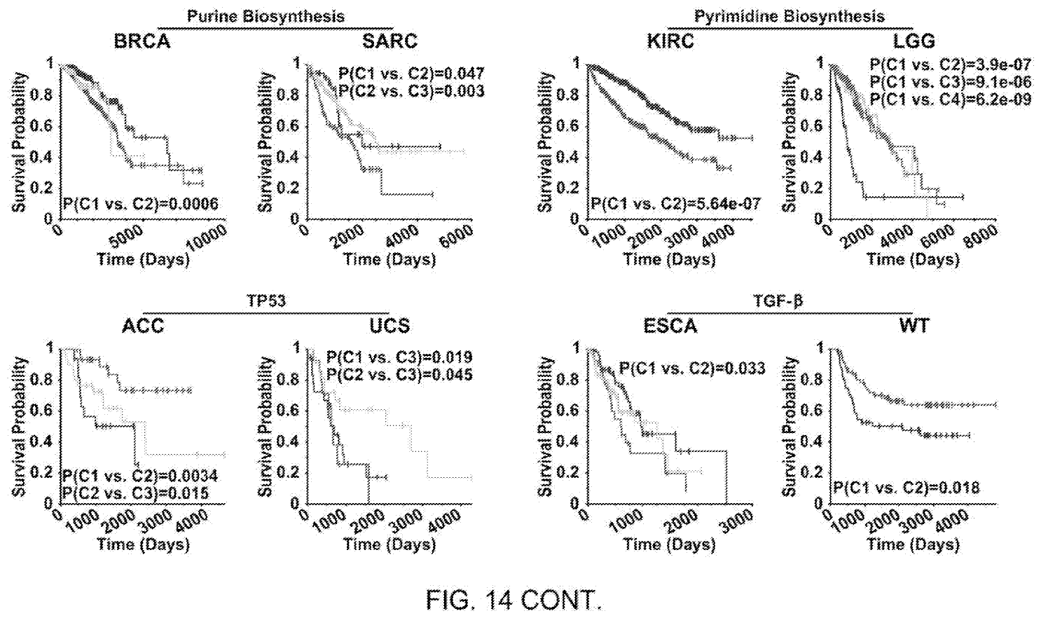

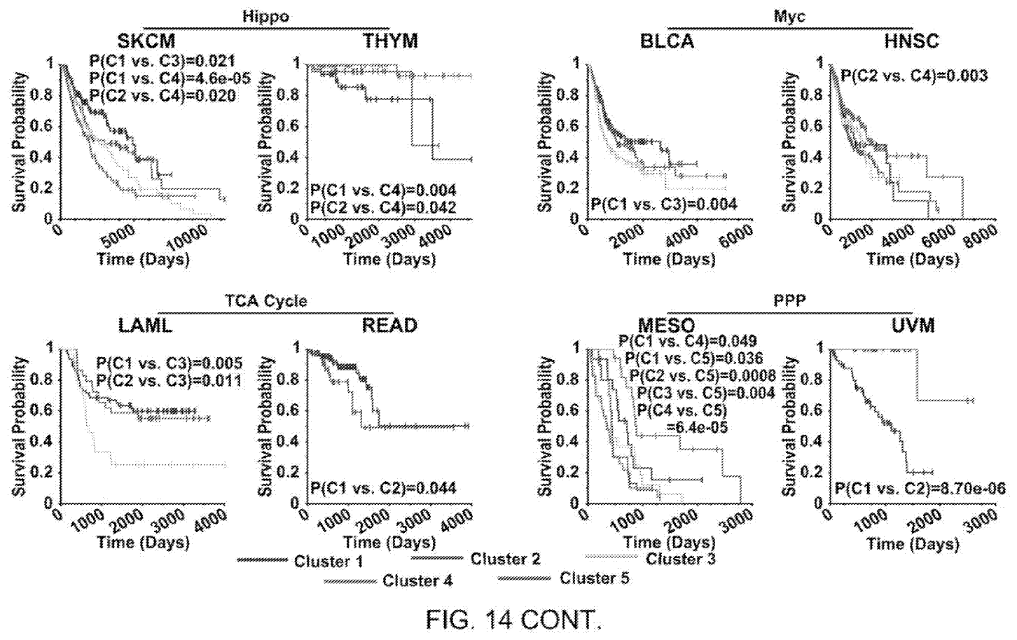

Transcripts for eight of the twelve cancer-related pathways shown in Table 1 and FIG. 14 were obtained from Sanchez et al. Transcripts representing the Pentose Phosphate Pathway and Purine and Pyrimidine Biosynthetic Pathways were selected because of their roles in providing critical anabolic precursors for nucleic acid synthesis. Finally, TCA Cycle transcripts were selected because oxidative phosphorylation is often altered or otherwise impaired in cancer cells as they redirect their utilization of glucose, fatty acids and glutamine. RNA expression data (FPKM-UQ) data were taken from the TCGA GDC PANCAN dataset and accessed through the UCSC Xenabrowser. Expression values were initially stored as the base-two logarithm of the incremented-βy-one FPKM-UQ value. The inverse of this transformation was applied to the values to obtain the true FPKM-UQ values.

(2) Depiction of Cancer Pathway Transcript Patterns

Prior to visualization via t-SNE, RNA expression data for all samples of each cancer type were centered and normalized for each pathway. Briefly, every primary tumor sample was assigned an “expression vector” in n-dimensional space for each pathway, where n was equal to the number of genes in the pathway and each element of the vector was equal to the FPKM-UQ expression value of the gene. For each cancer type, the associated expression vectors were centered and normalized by subtracting by the mean value of all vectors associated with samples of the cancer type. The centered vectors were then normalized by their magnitudes. The result was that all centered expression vectors were projected onto a hyper-sphere in n-dimensional space. For each cancer type and each pathway, the vectors on this hypersphere were the input to t-SNE. t-SNE analyses of each pathway's transcript patterns were performed using Tensorboard in three dimensions to maximize the appreciation of the compactness and separateness of the resulting clusters. Multiple t-SNE runs were executed with perplexities ranging between 5 and 22, and learning rates of either 1, 10, or 100. The combination of parameters that yielded the most consistent and compact cluster as determined by inspection were selected for further validation by multiple runs. For the final selected parameters t-SNE was run for at least 2500 iterations and until the t-SNE stabilized. After embedding, the number of clusters was recorded. Cluster members were then specified using a Gaussian mixture model (GMM) implemented through MATLAB's ‘fitgmdist’ and ‘cluster’ functions (see Methods and Table 3). All such groups are referred to hereafter as “t-SNE clusters”.

(3) Comparing t-SNE Clusters

Clinical and survival data for TCGA cancer cohorts were accessed using the UCSC Xenabrowser under the data heading “Phenotypes”. Kaplan-Meier survival curves of tumors in each t-SNE cluster were compared using Mantel-Haenszel (log-rank) methods through the “Matsury” function on the MATLAB file exchange and confirmed in Graphpad Prism 7. Categorical clinical variables were compared between clusters of tumors with chi-squared tests[MJA1]. Continuous variables which were normally distributed were compared with t-tests assuming heteroskedasticity, and non-normally-distributed variables were compared with Wilcoxon sign-rank tests. All statistical tests were two-tailed.

(4) Random Forest Analyses

To identify the genetic features that differed the most among different clusters, a random forest classifier model was employed through MATLAB's ‘TreeBagger’ function in the ‘Statistics and Machine Learning Toolbox’, with ‘NumTrees’ equal to 100, ‘OOBPredictorImportance’ turned on, ‘NumPredictorsToSample’ set to ‘all’, and ‘PredictorSelection’ set to ‘interaction-curvature’. The importance of the transcripts in distinguishing the clusters from one another were indicated by the ‘OOBPermutedPredictor’ field of the object returned by the ‘TreeBagger’ function.

(5) Comparison of T-SNE Clusters with Hierarchical Clusters

To investigate the relationship between t-SNE clusters and the entire expressed protein-coding genome, a small group of cancers were selected for full transcriptome visualization by hierarchically clustered heat maps. To this end, next-generation RNAseq heat maps of the cancers of interest were downloaded from the TCGA Next-Generation Heat Map Compendium. The platform “RNA Expression” was selected and heat map type selected as “Gene/Probe vs Sample”. The tumor samples represented in this heat map had a high degree of overlap with the samples used in tSNE. Samples were pre-divided into three-six hierarchical groups (abbreviated here as ‘Dendros’ to avoid confusion with the t-SNE clusters). For the selected cancers, the members of the Dendros were subdivided according to which t-SNE group with which they associated. Significance of survival differences between these groups within each Dendro was assessed in Graphpad Prism 7 using log-rank tests.

(6) Implementation of Clustering Algorithm

t-SNE clusters were specified using a Gaussian mixture model implemented through MATLAB's “fitgmdist” and ‘cluster’ functions. The default “K-means++” algorithm was used to set initial conditions in all cases. In some cases, the output t-SNE data were randomly perturbed by 5% of the radius of the smallest sphere that contained all the output points before clustering. The number of Gaussian components used was equal to the number of clusters previously identified. For each t-SNE profile, every combination of full or diagonal covariance matrices, shared or unshared covariance and the application or non-application of the aforementioned perturbation were iteratively tried when fitting the Gaussian mixture model, for a total of eight attempts with different parameter settings. The output that best preserved the unity of the clusters in the t-SNE were chosen for display in all figures. Finally, the aforementioned perturbation was applied to the actual output t-SNE scatterplot displayed in the figures in cases where clusters were so dense as to prevent its individual component members from being readily visualized The parameters used for each tSNE are listed in Table 3.

b) Results

(1) Transcript Expression Patterns from Cancer-Related Pathways Predict Survival

Cancers are characterized by qualitative and/or quantitative gene expression changes, which weaken normal constraints on cell growth, survival and metabolism. These changes are usually clonal and arise sequentially in multiple cooperating pathways during tumor evolution. Each change deregulates its respective pathway and imparts a selective growth and/or survival advantage. The cataloging of these alterations has played an ever-increasing roll in tumor classification, prognosis and therapeutic optimization.

Using t-SNE profiling, RPT t-SNE pattern differences were observed among human cancers that are recurrent, specific for each cancer type and distinguishable from the RPT t-SNE patterns of the tumors' tissues of origin. Multiple tumor-specific RPT t-SNE clusters were usually observed and in seven tumor types, were predictive of long-term survival. Importantly, RPT t-SNE patterns were largely independent of their absolute expression levels.

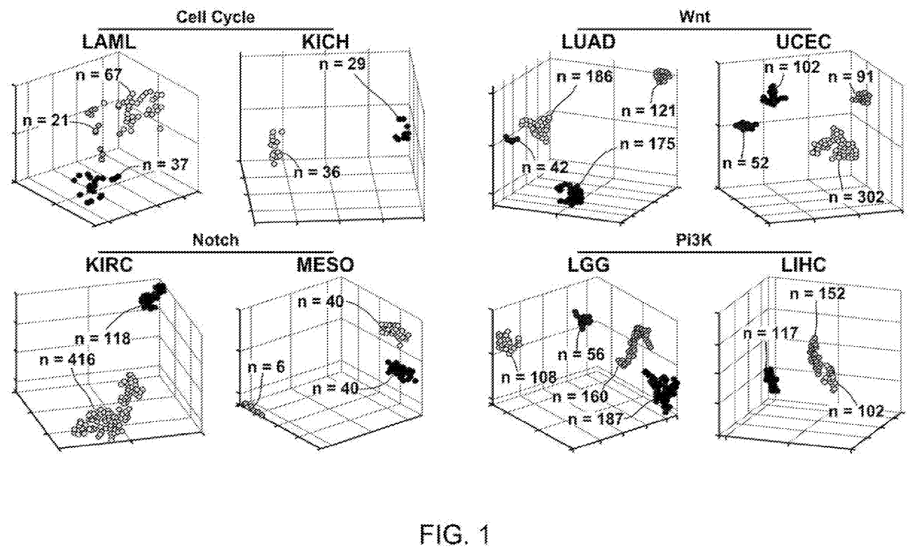

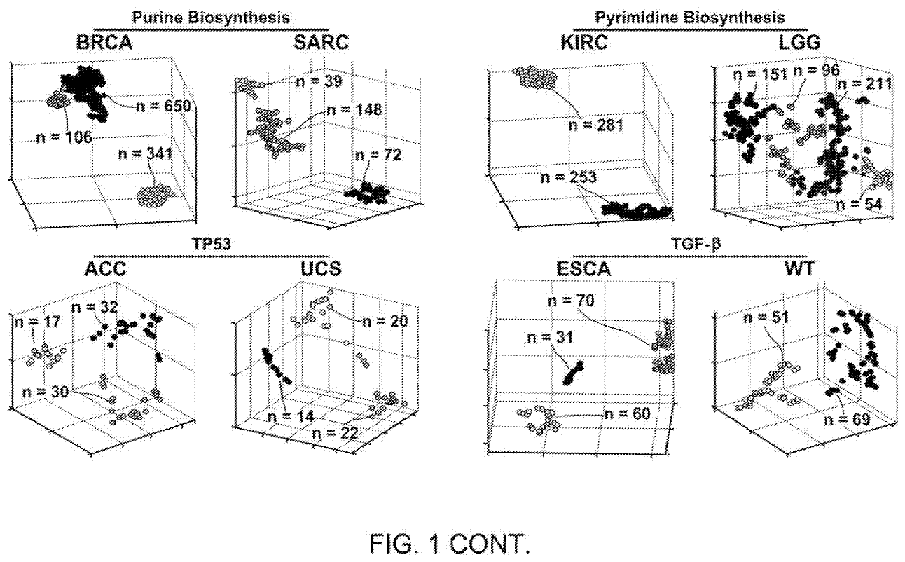

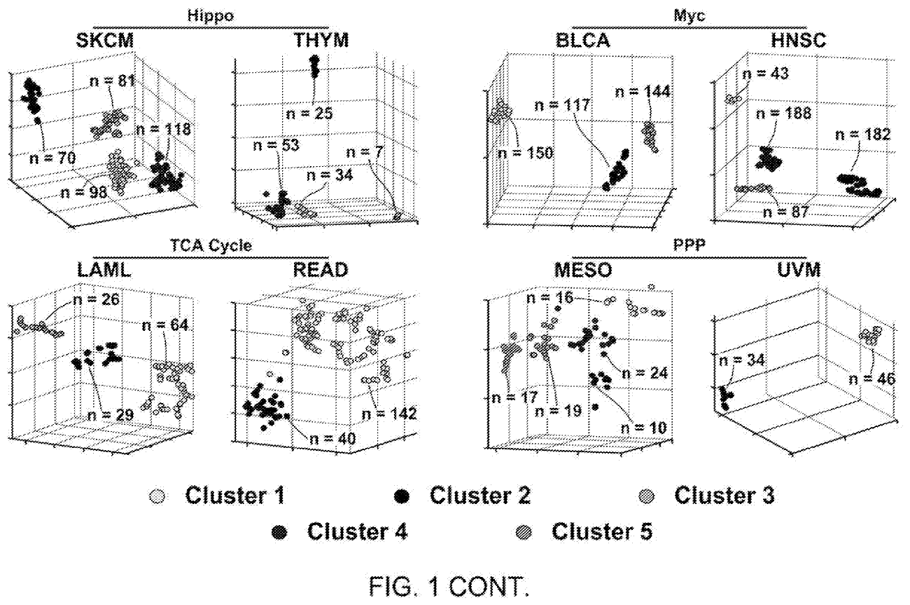

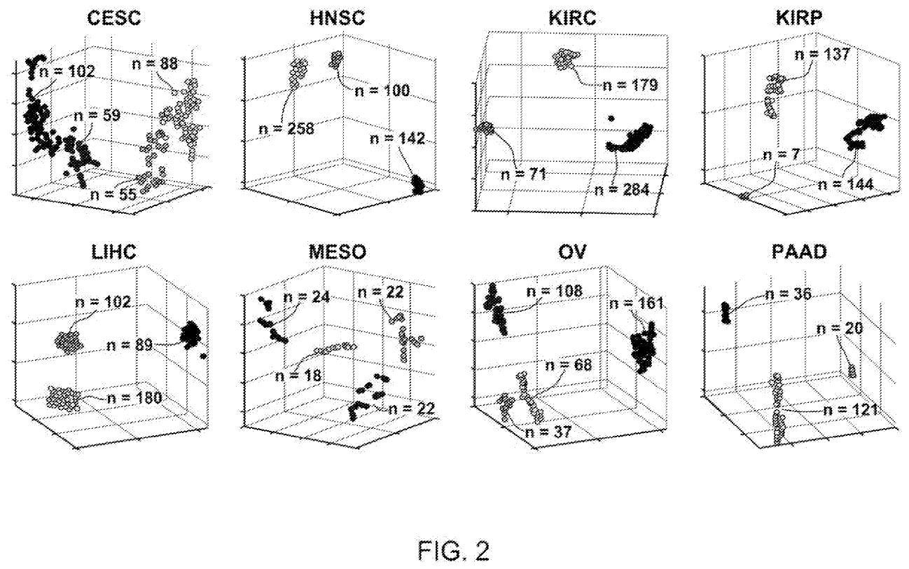

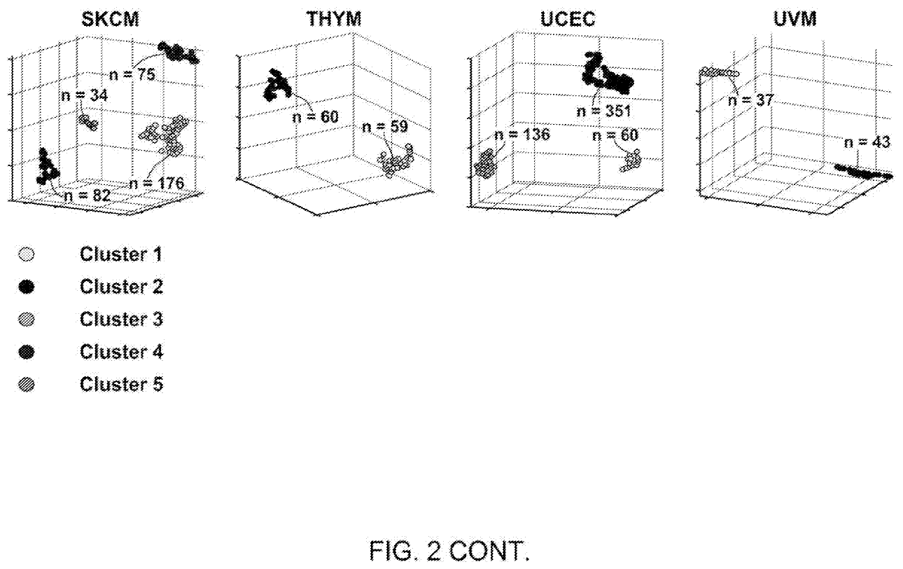

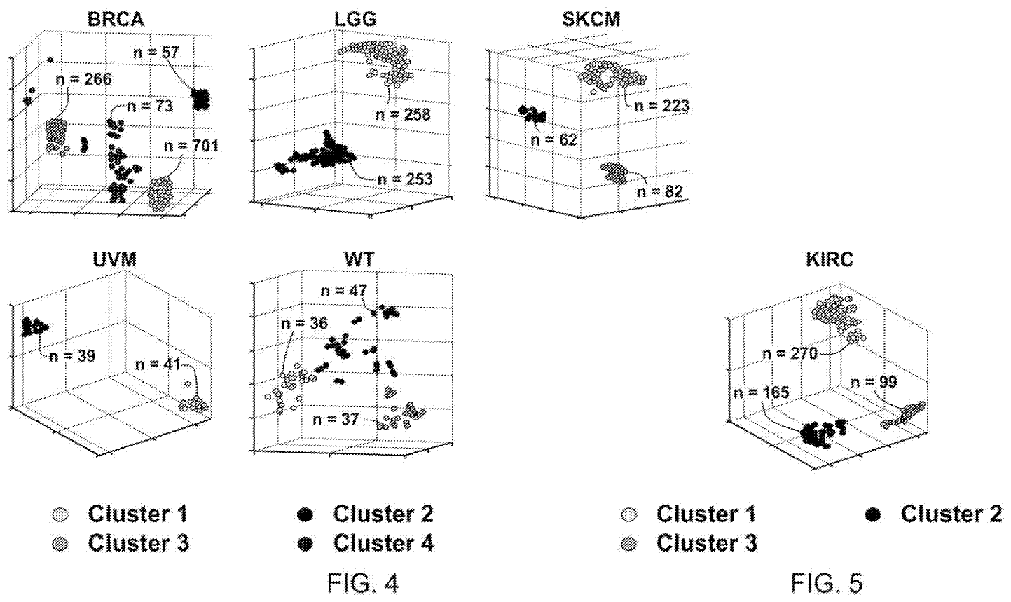

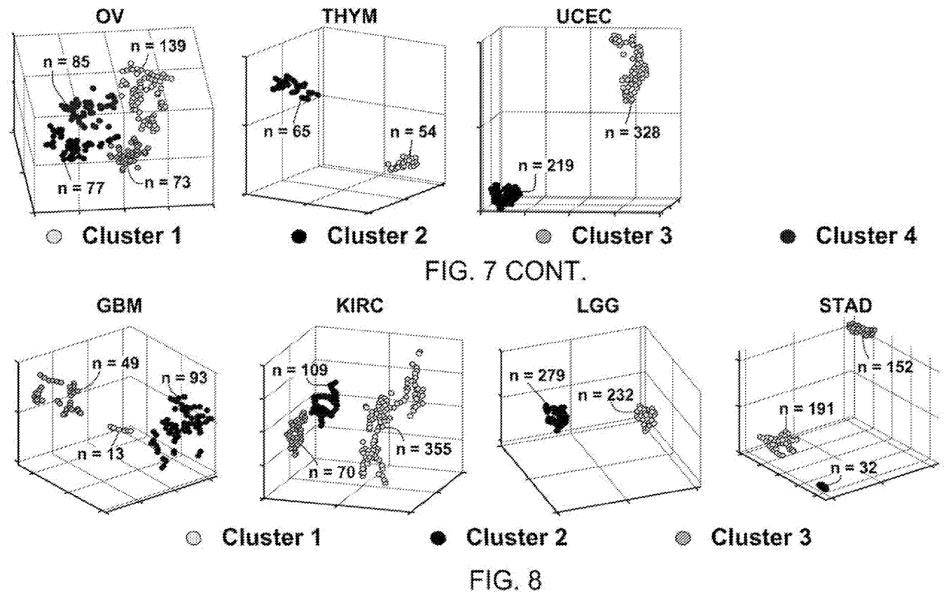

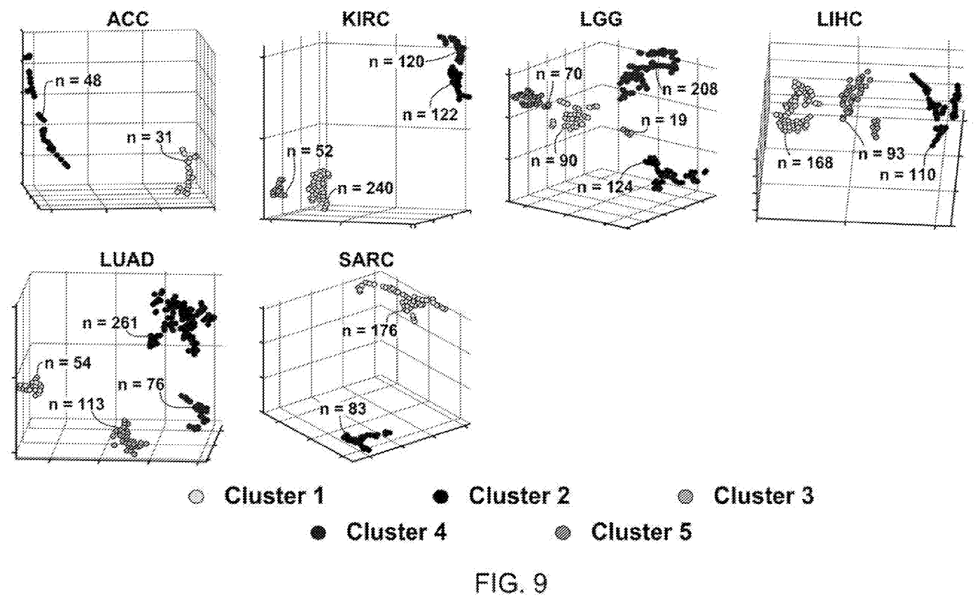

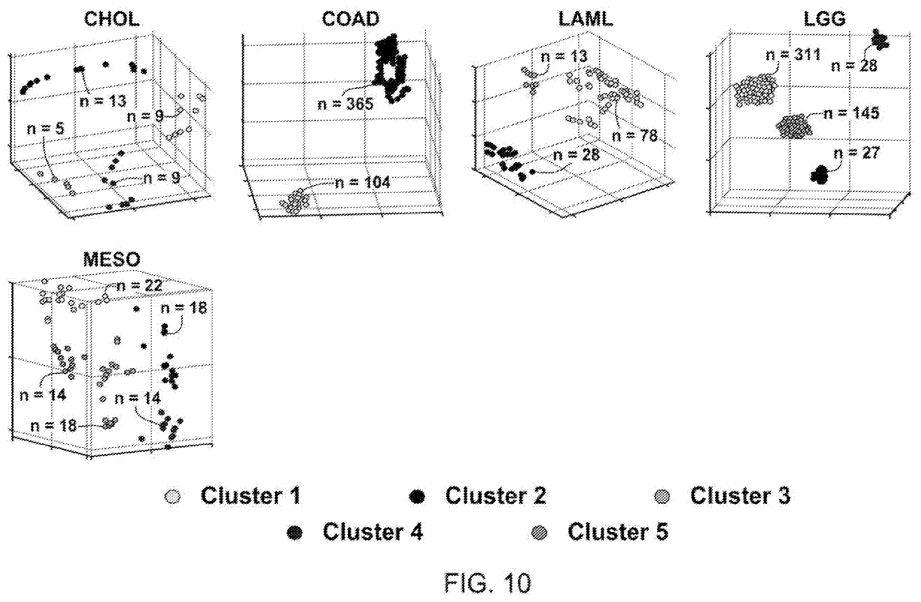

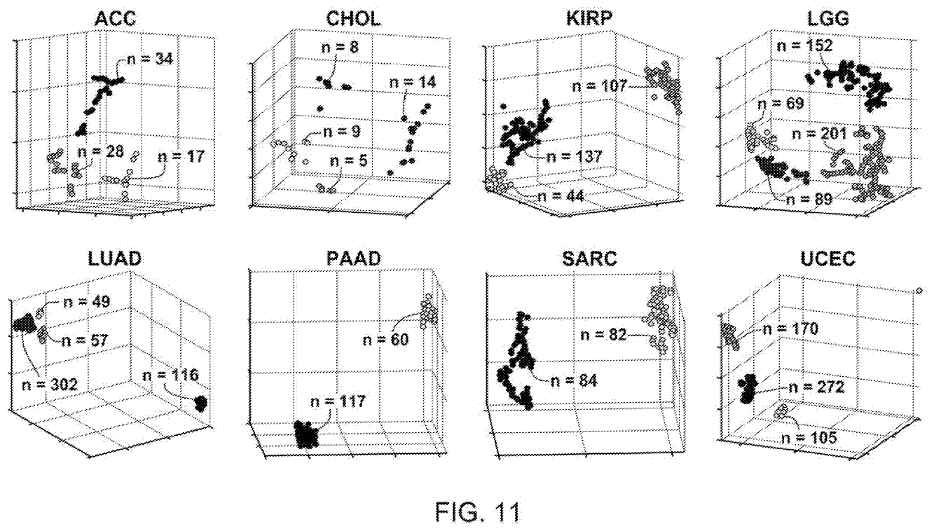

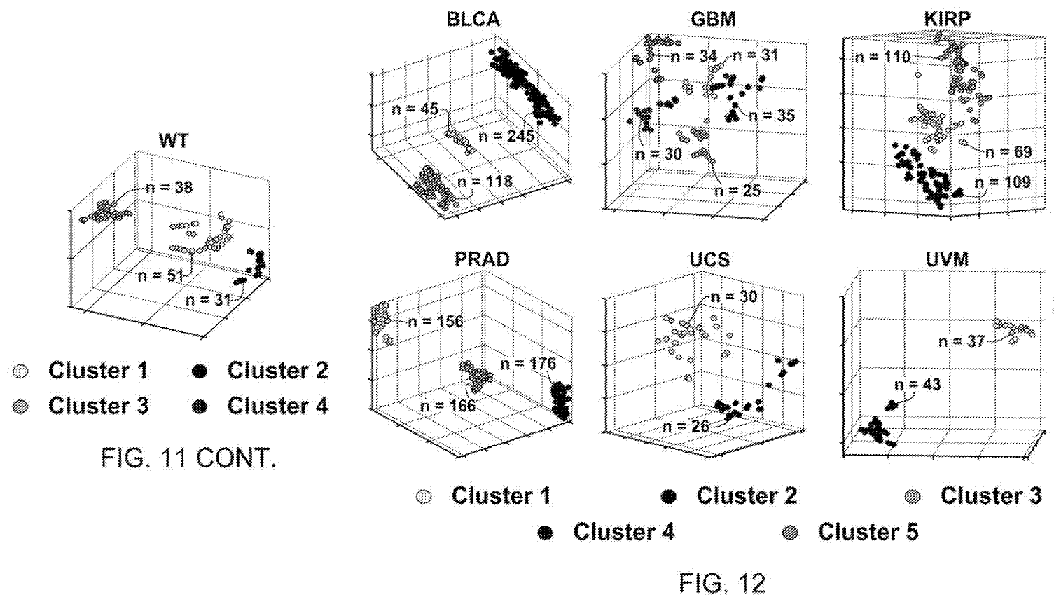

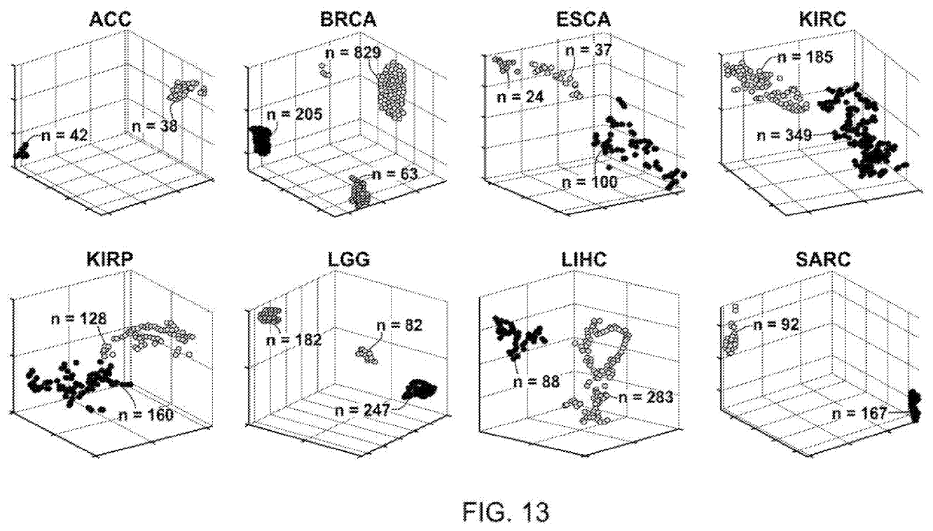

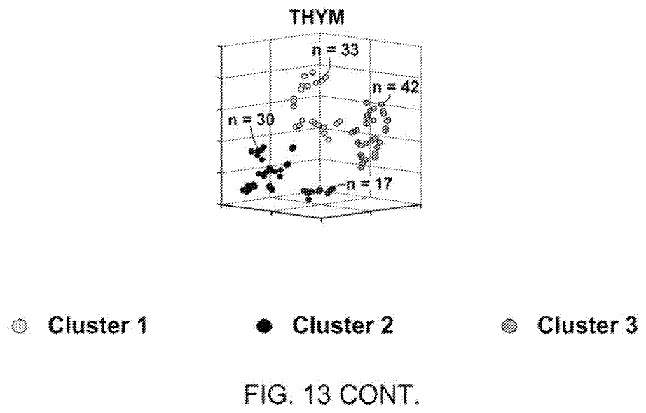

The above findings raised the question of whether altered gene expression patterns in other cancer-related pathways could also predict survival and, if so, whether combinations of these pathways could perhaps improve their prognostic utility. Therefore a “core” group of 212 transcripts representing 12 cancer pathways (CP) with well-defined roles in cancer cell proliferation was assembled, survival and metabolism as a result of recurrent dysregulation of some of their component members (Table 1). In 10,227 samples from TCGA representing 34 distinct cancer types, t-SNE identified distinct, tumor type-specific clusters of transcript patterns for each pathway. In virtually all cases, tumor groups contained more than a single such cluster for each pathway thus indicating heterogeneity in each family's cancer pathway transcript (CPT) expression patterns (FIGS. 1, 2, 3, 4, 5, 6, 7, 8, 9, 10, 11, 12, and 13).

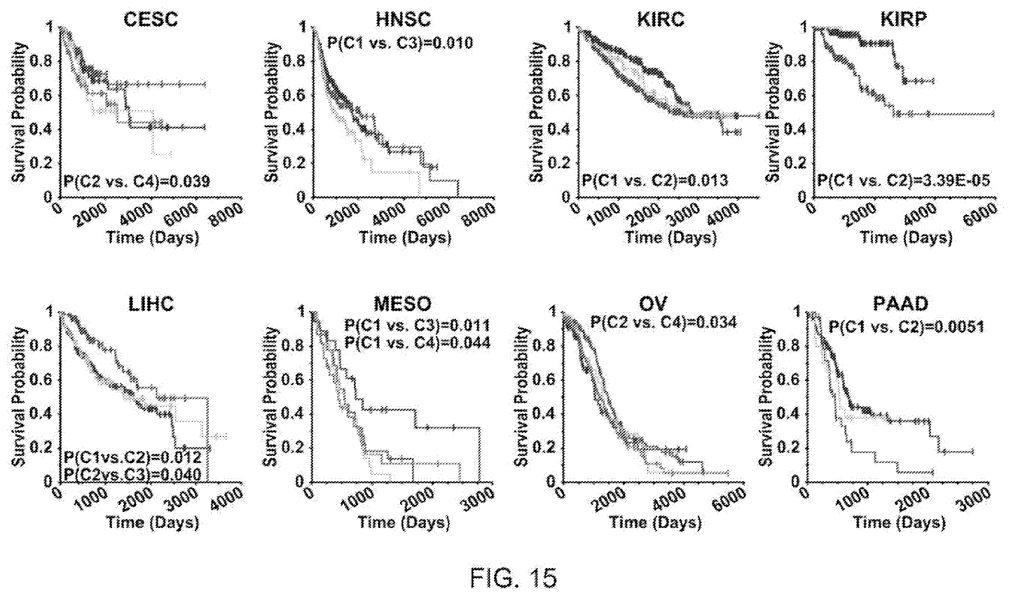

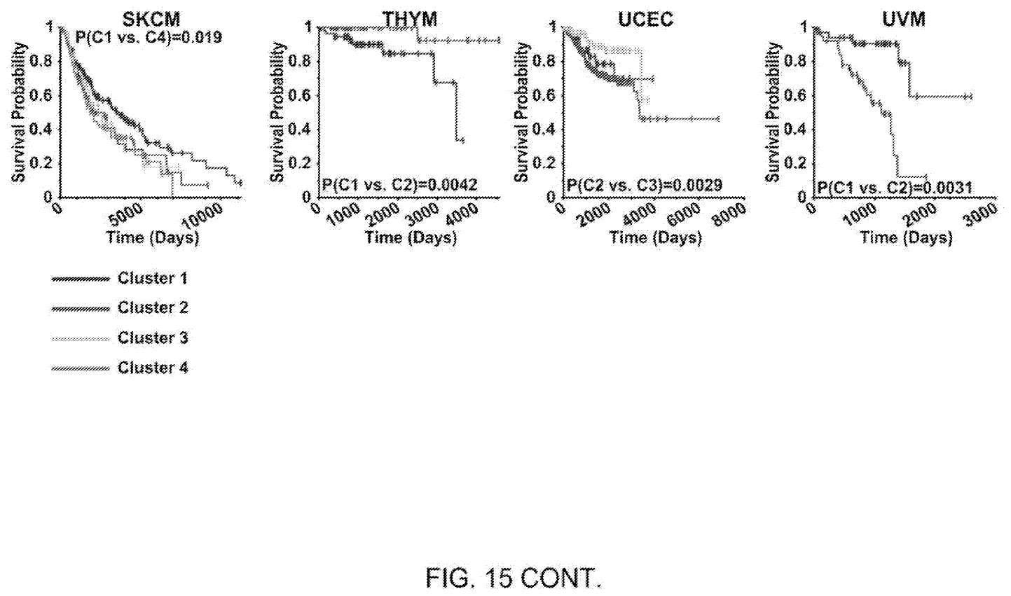

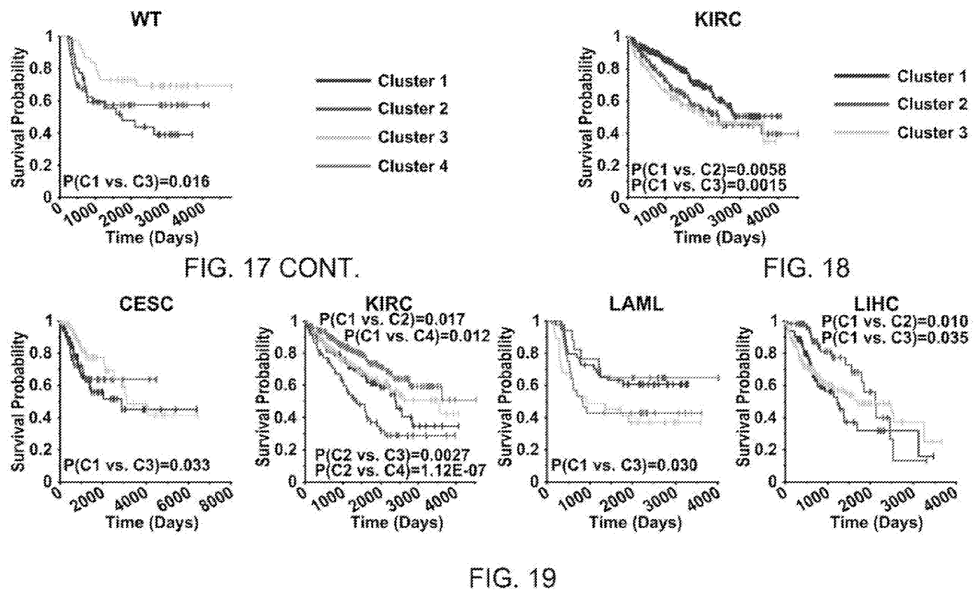

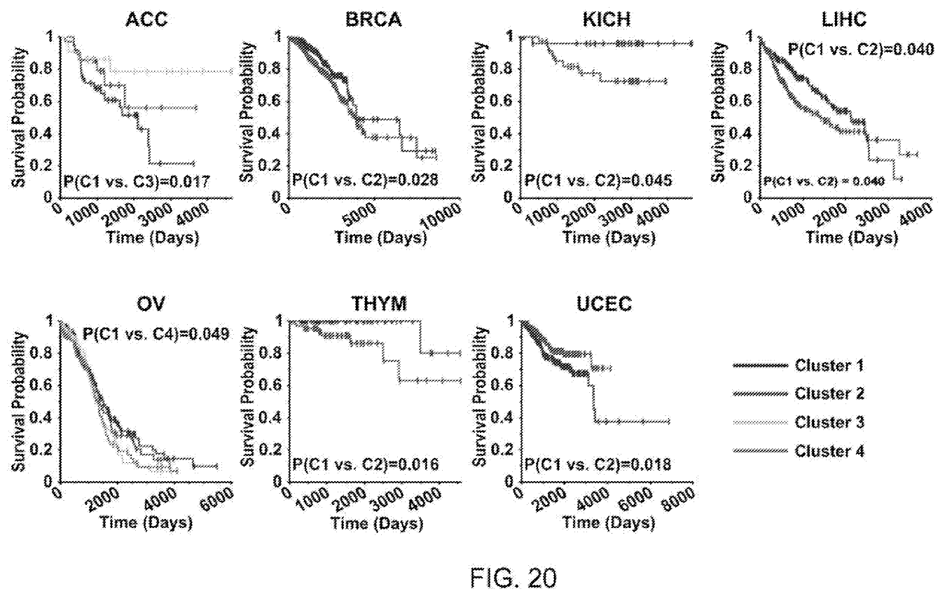

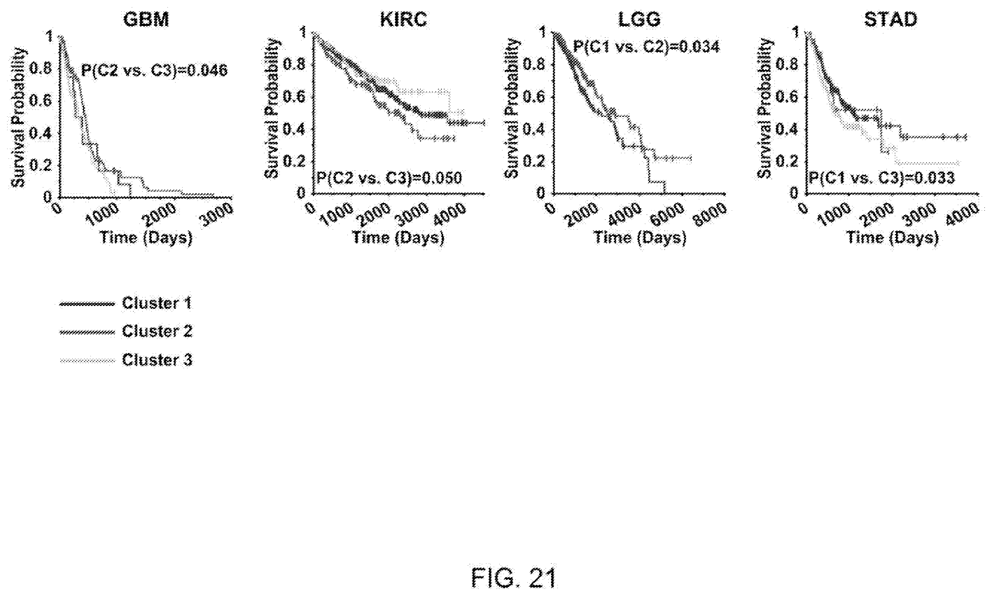

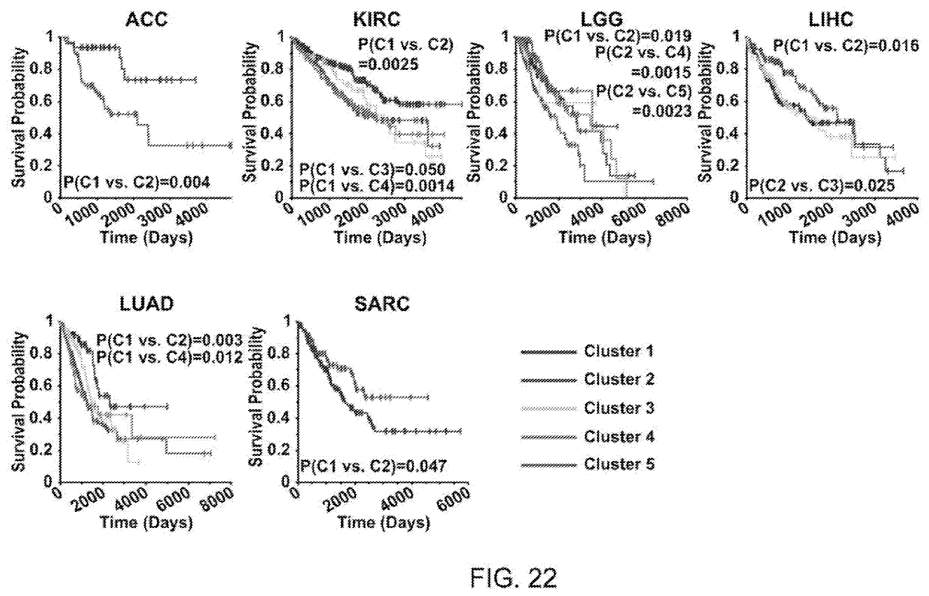

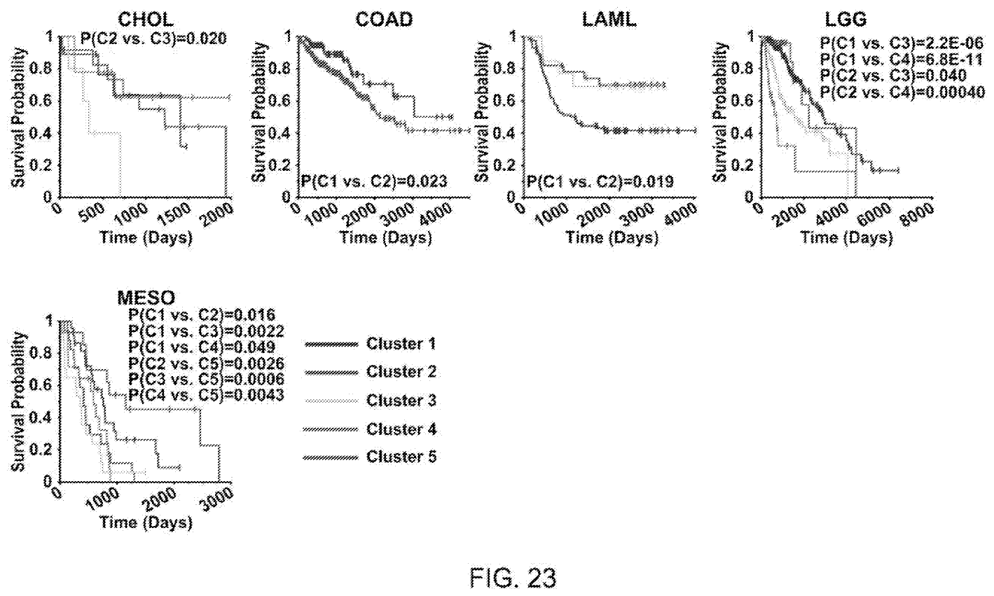

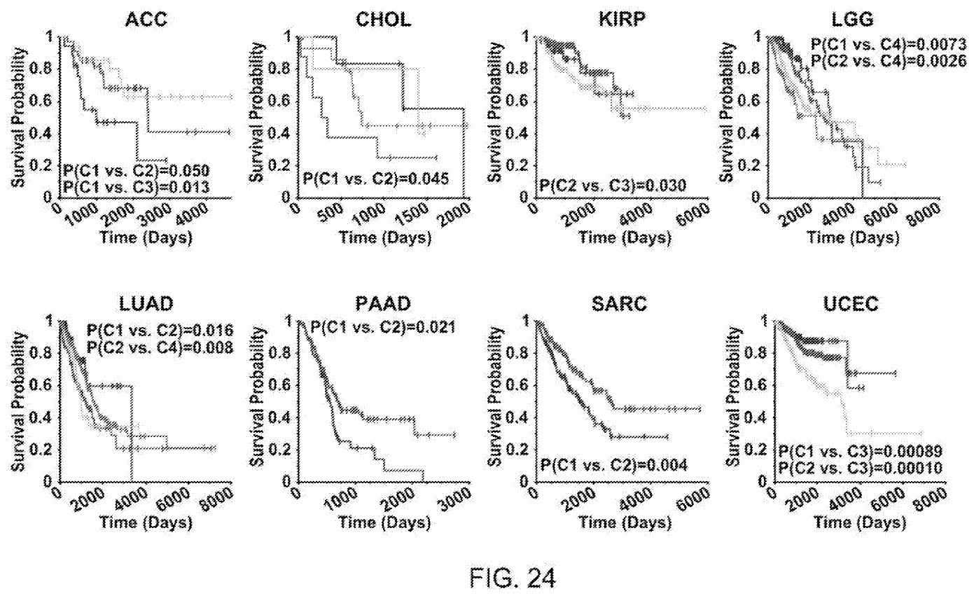

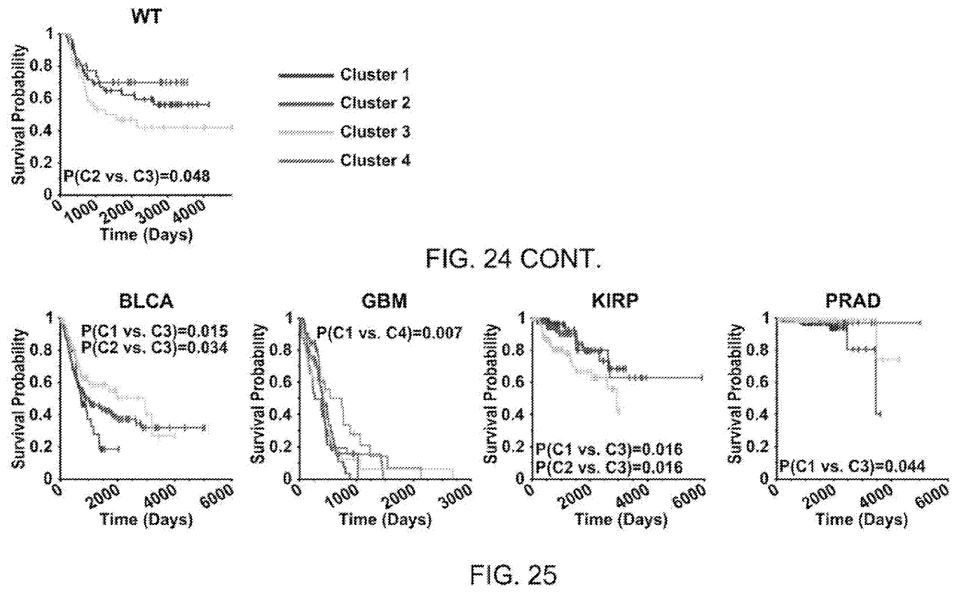

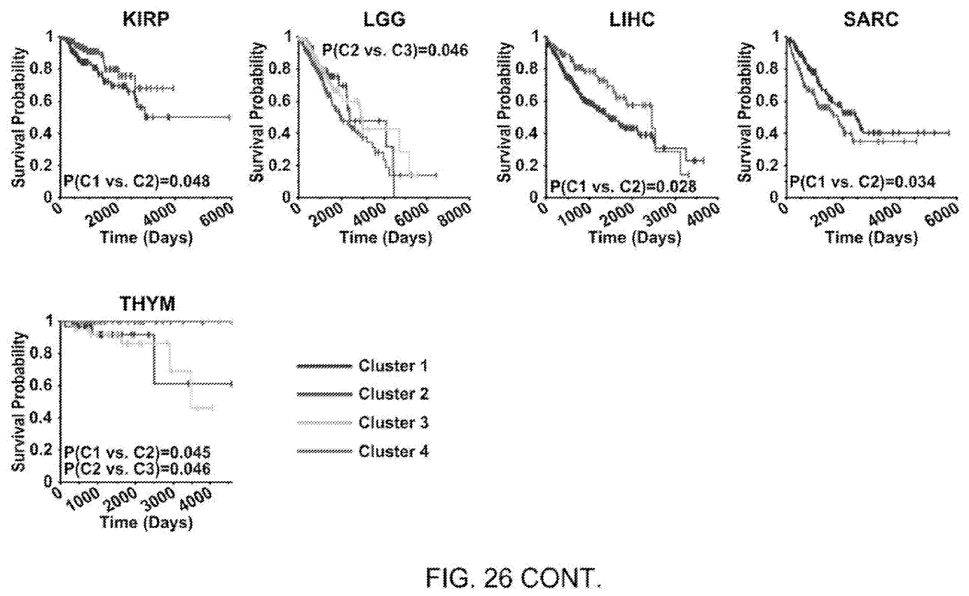

Many t-SNE clusters shown in FIGS. 1, 2, 3, 4, 5, 6, 7, 8, 9, 10, 11, 12, and 13 were associated with significant survival differences (FIGS. 14, 15, 16, 17, 18, 19, 20, 21, 22, 23, 24, 25, and 26). Indeed, the expression patterns of individual pathway's transcripts correlated with survival in 3-14 cancer types, comprising 9.6-38.9% of the entire TCGA population (FIGS. 14, 15, 16, 17, 18, 19, 20, 21, 22, 23, 24, 25, 26, and 27). Considerable overlap was also found among the different pathways for individual tumor types. For example, transcript expression patterns from the Wnt, Pyrimidine Biosynthesis, Myc and TCA Cycle pathways were all highly predictive of survival in clear cell renal cancer (KIRC) (P<0.0001 for each). Similarly, transcript expression patterns for PI3K, Purine Biosynthesis, Hippo and Myc Pathways were each highly predictive of survival for low-grade gliomas (<0.0001 for each). In contrast only a single pathway's t-SNE profile was predictive of survival in glioblastoma multiforme (GBM) (TP53 pathway), ovarian serous cystadenocarcinoma (OV) (cell cycle), rectal adeno-carcinoma (READ) (cell cycle pathway) and uterine carcinosarcoma (UCS) (TP53 pathway) (0.01<P<0.05 in all cases). Additionally, survival for all cancers could be predicted by t-SNE profiles from a mean of 3.7 pathways. This ranged from 9 pathways for low-grade gliomas and clear cell kidney cancer to a single pathway each for colon, prostate, rectal and prostate cancers (FIG. 27). Nevertheless, no t-SNE pattern was predictive of survival in squamous cell lung cancer, diffuse large B-cell lymphoma (DLBC), pheochromocytoma/paraganglioneuroma (PCPG), or testicular germ cell tumor (TGCT) collectively comprising 8.6% of the entire TCGA population. Thus, at least one pathway accurately predicted survival in 30 of 34 cancer groups, comprising 91.4% of the entire TCGA tumor population (FIG. 27).

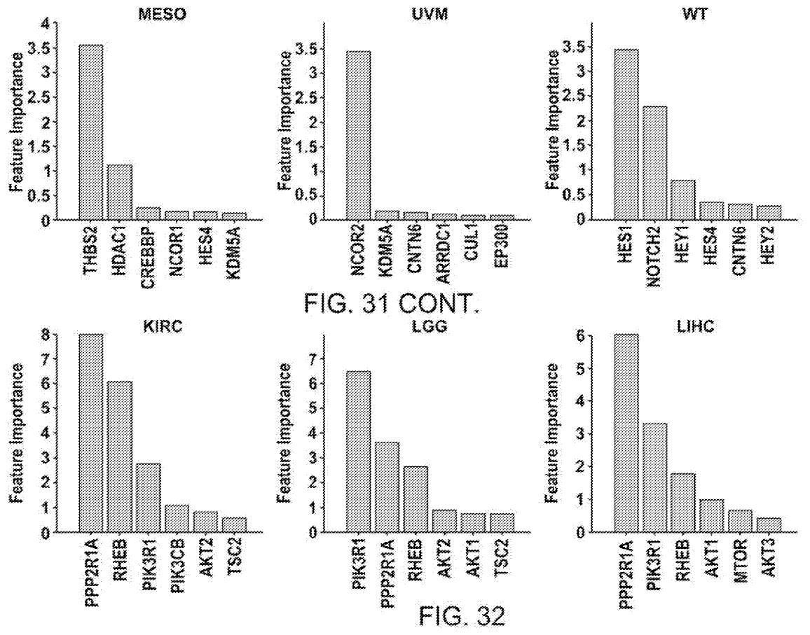

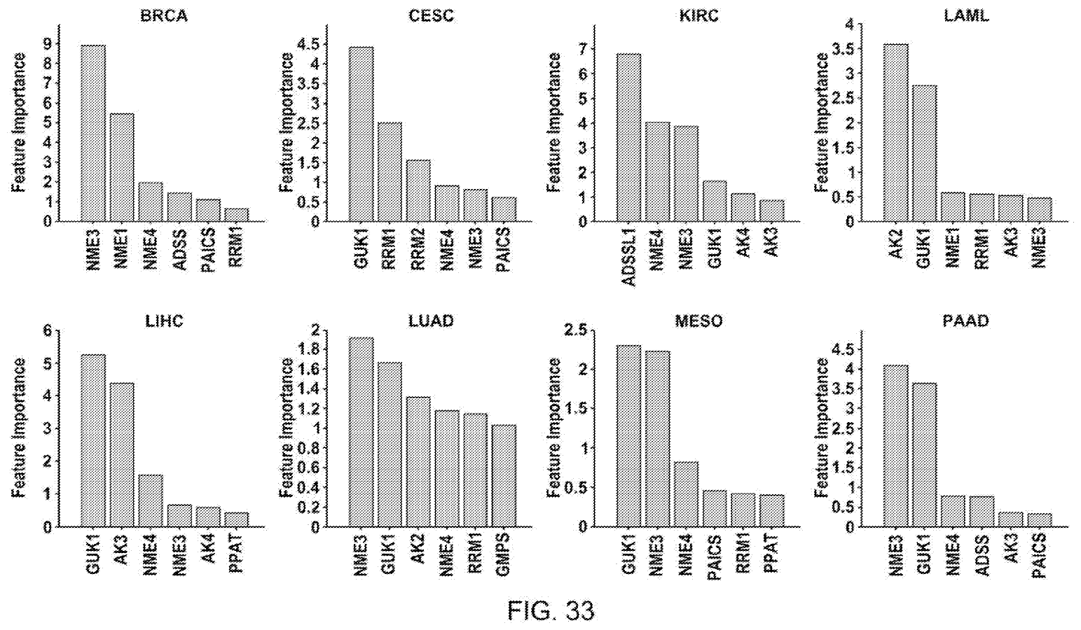

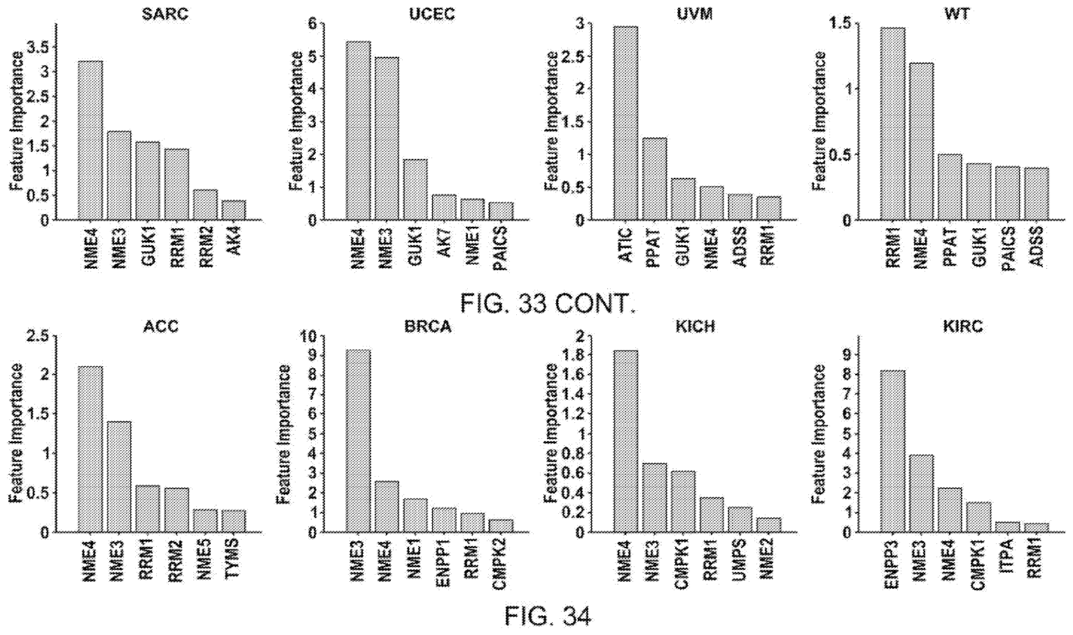

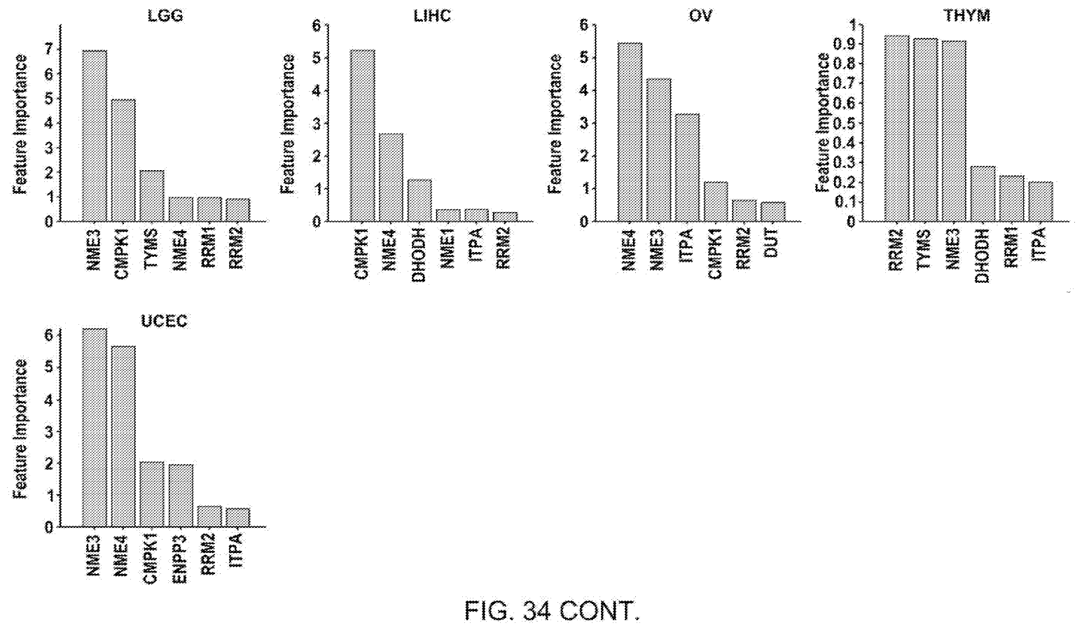

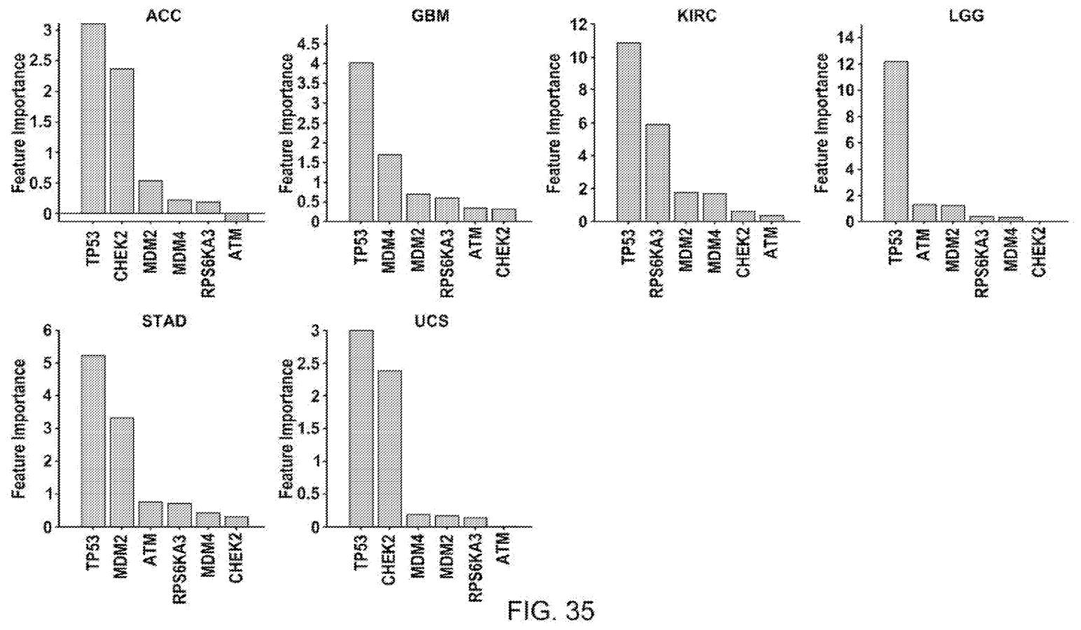

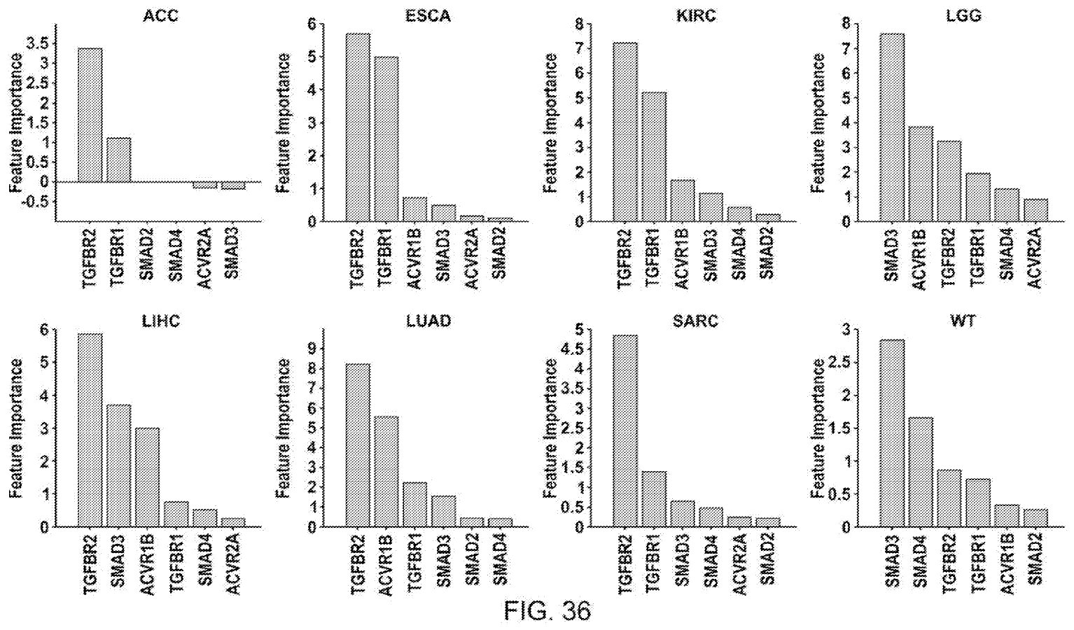

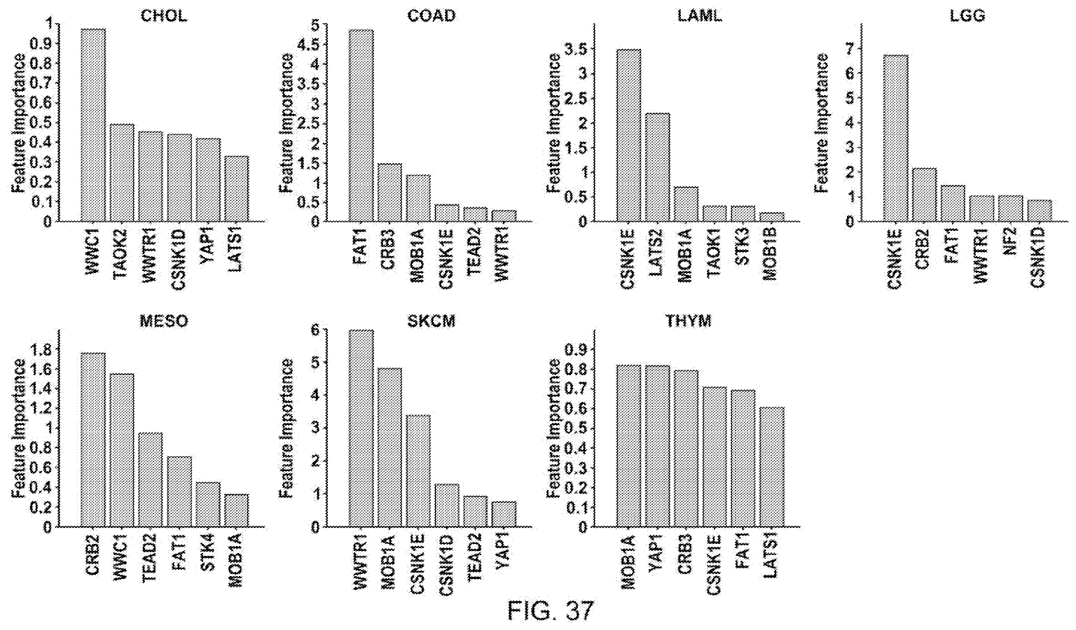

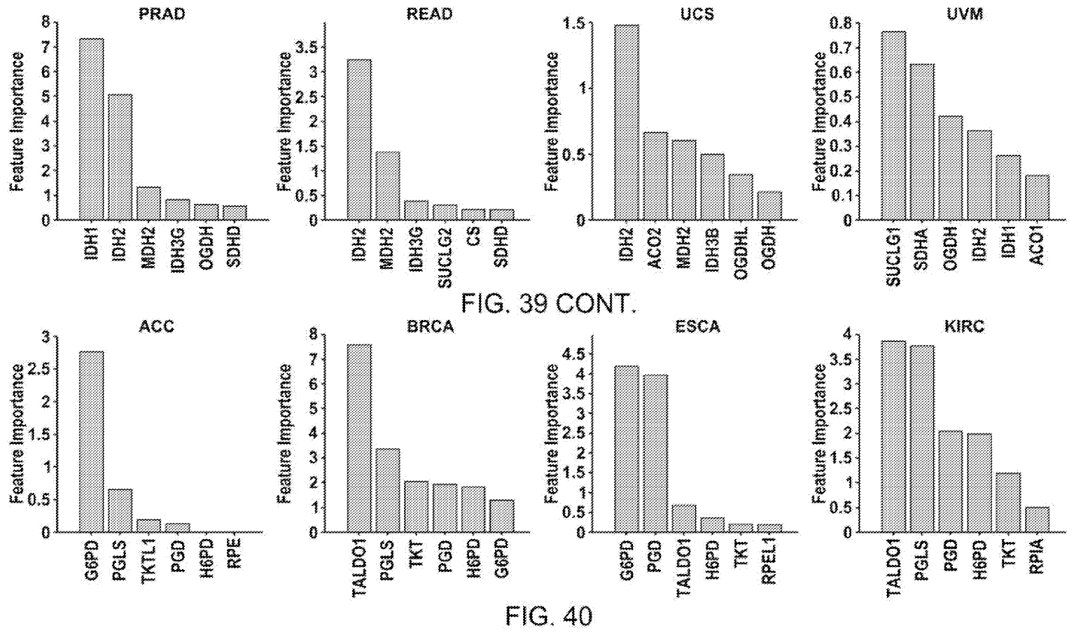

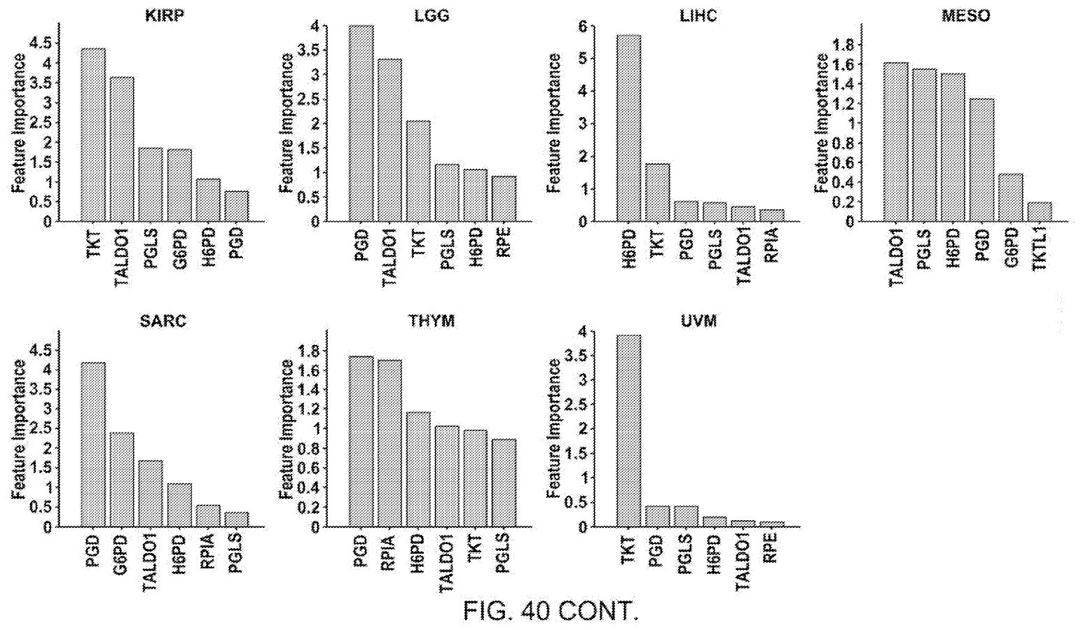

Certain RPT transcripts disproportionately shape t-SNE clusters across a broad range of tumor types. Therefore, a Random Forest classifier was applied to identify transcripts in each of the above twelve cancer pathways that were the most important in determining the t-SNE profiles across all cancers. These were relatively few in number, ranging from as few as 1-2 to as many as 4-6 depending both on the tumor type and the specific pathway (FIGS. 29, 30, 31, 32, 33, 34, 35, 36, 37, 38, 39, and 40). Thus, a much smaller subset of the original 212 member collection, comprising as few as 60 cancer pathway transcripts (CPTs), contributed disproportionately and recurrently to the t-SNE profiles of most cancers.

(2) t-SNE Analysis and Whole Transciptome Profiling can Complement One Another and Add Additional Predictive Value

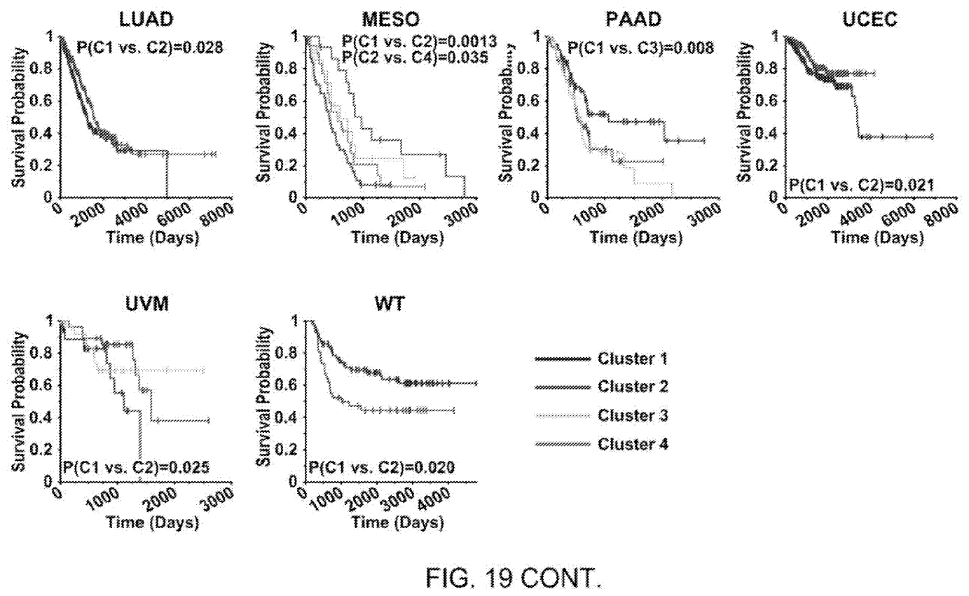

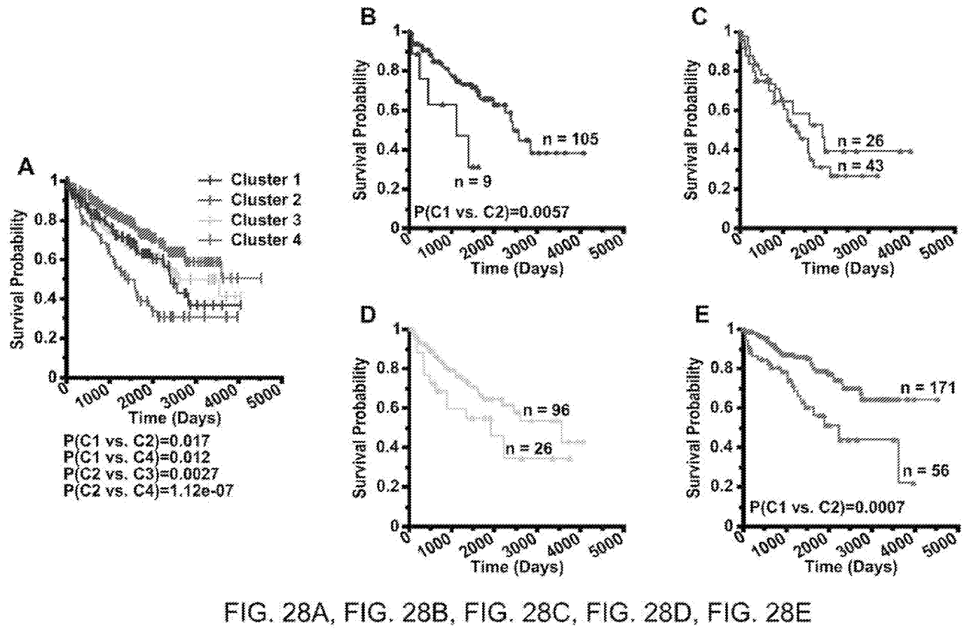

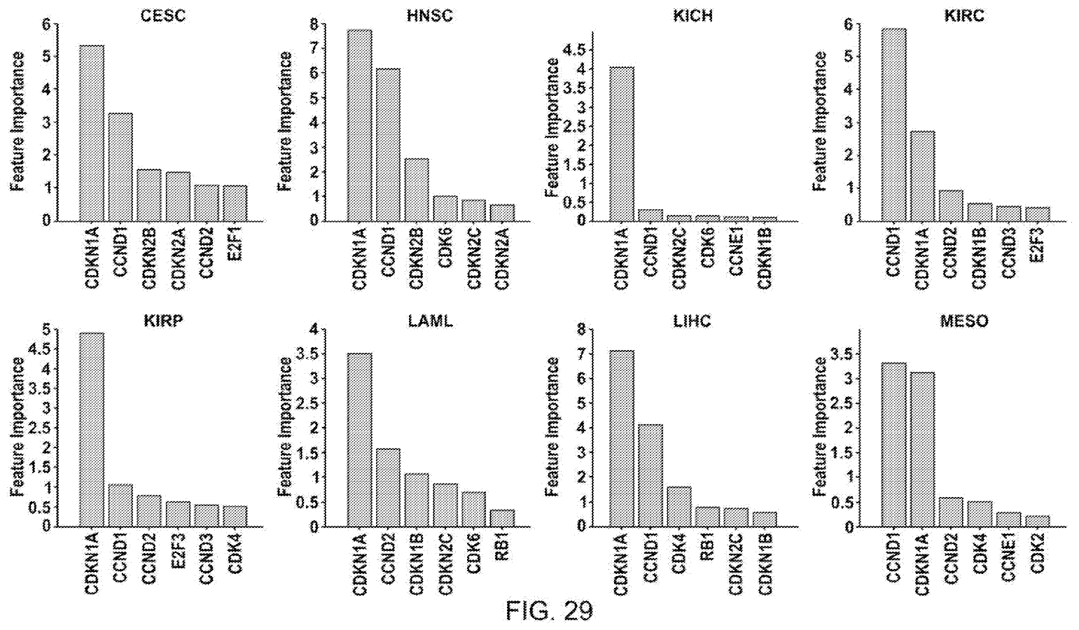

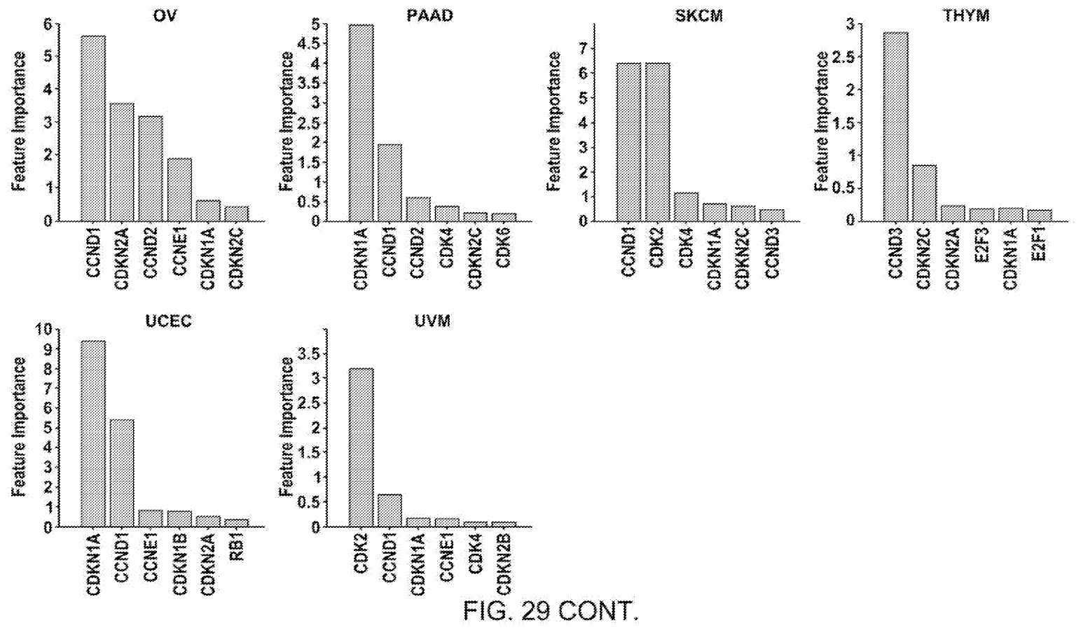

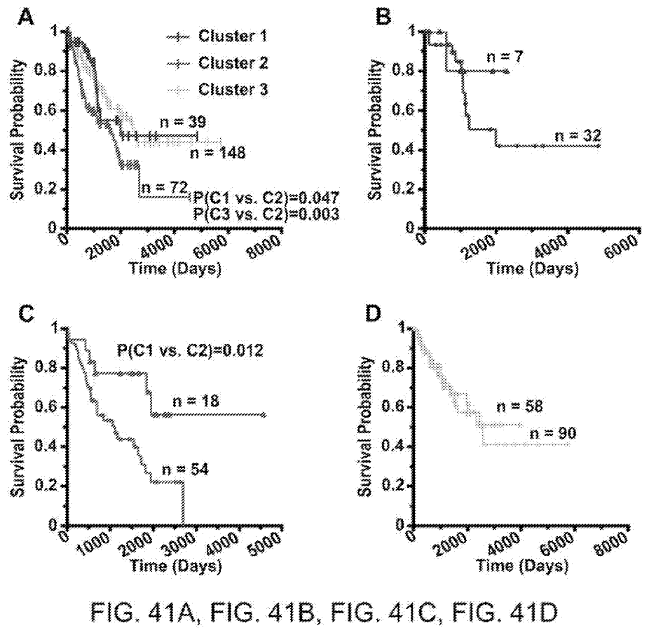

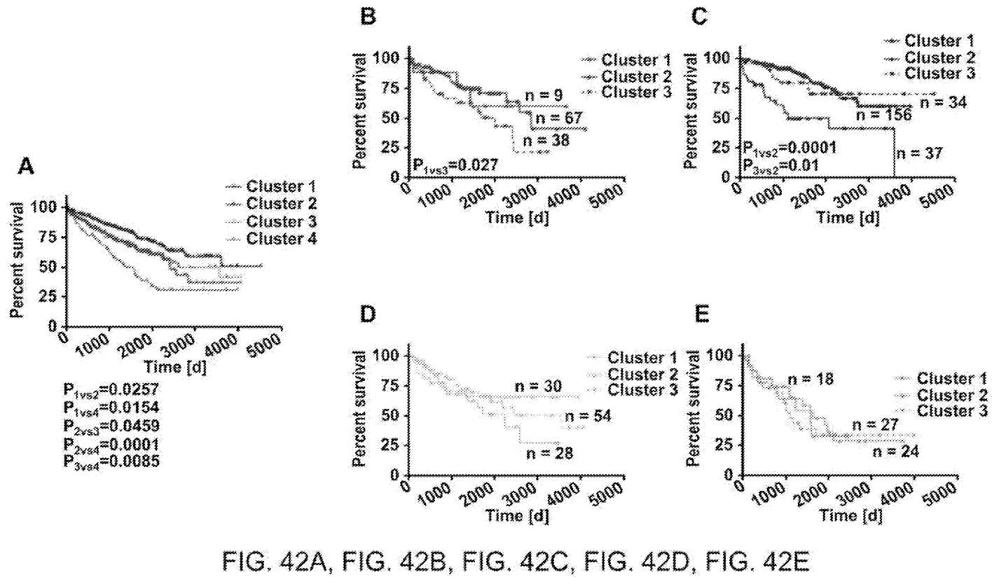

Because t-SNE profiles for more than one pathway correlated with survival in 25 of 34 cancers (FIG. 27), it was asked whether a second, sequential analysis performed on an initial set of t-SNE clusters could contribute additional predictive power. FIG. 28A shows the original Kaplan-Meier survival curves of the 4 patient cohorts (Clusters 1-4) with clear cell kidney cancer profiled with Purine Biosynthesis Pathway transcripts (FIG. 19). Subsequent t-SNE profiling with Notch Pathway members allowed a further subdivision of Clusters 1 and 2. Cluster 1, with relatively poor prognosis (median survival=2419 days), could be further sub-divided into a large sub-group with slightly longer median survival (2564 days) and a smaller sub-group with a particularly poor median survival of 1111 days (P=0.0057) (FIG. 28B). Cluster 4, had the best overall survival with a median survival of >3700 days and could also be subdivided into two groups with median survivals of >4700 days and 2241 days, respectively (P=0.0004) (FIG. 28E). Neither Clusters 2 or 3 could be further subdivided (FIGS. 28C and 28D). At least two additional examples of initial t-SNE clusters (generated from sarcomas and head and neck squamous cell cancers) that could be further sub-classified with a second pathway's transcripts are shown in FIGS. 41, 42, and 43).

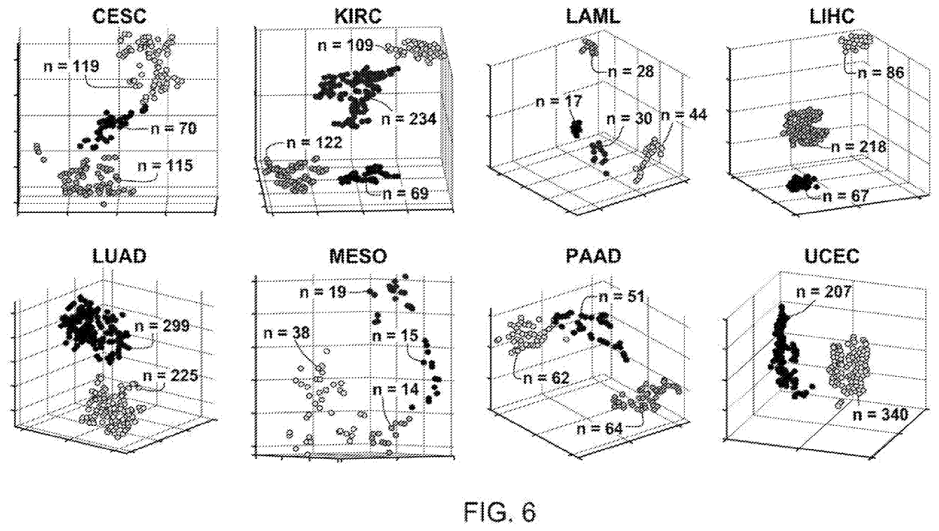

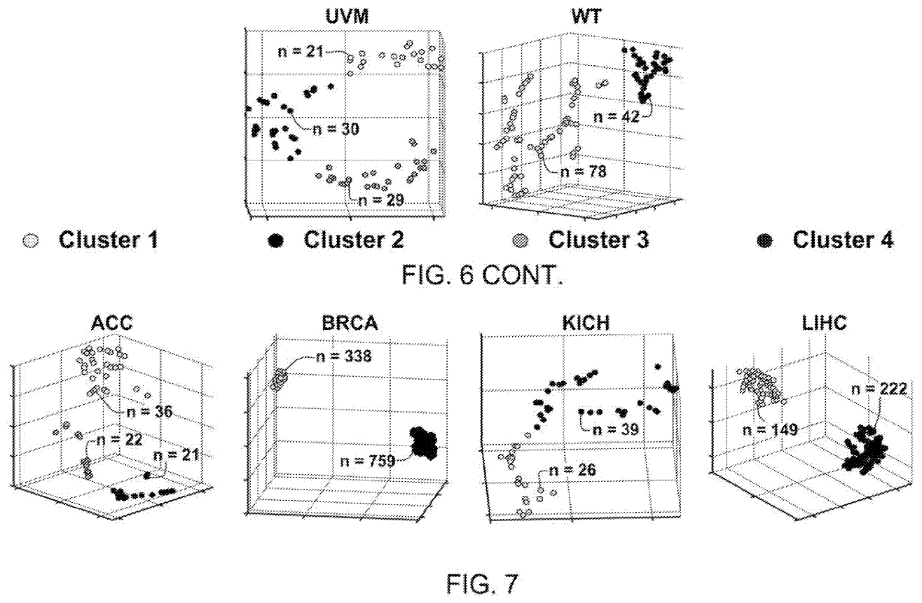

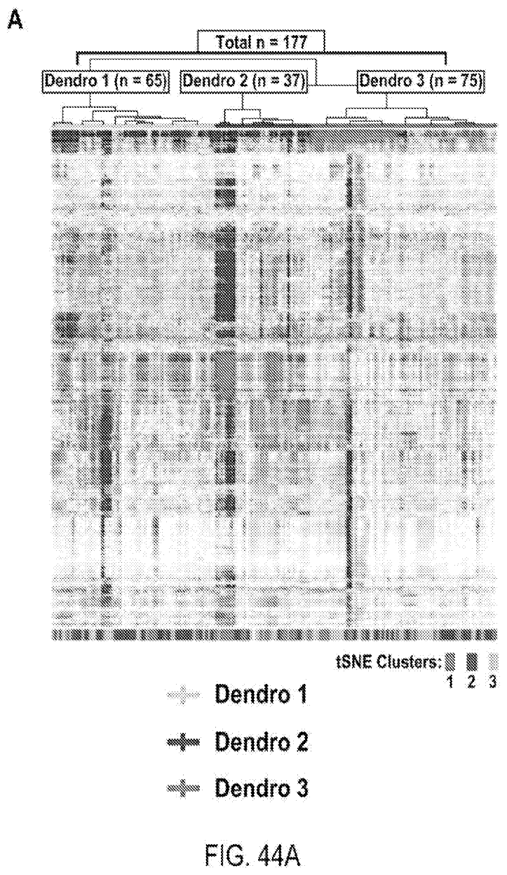

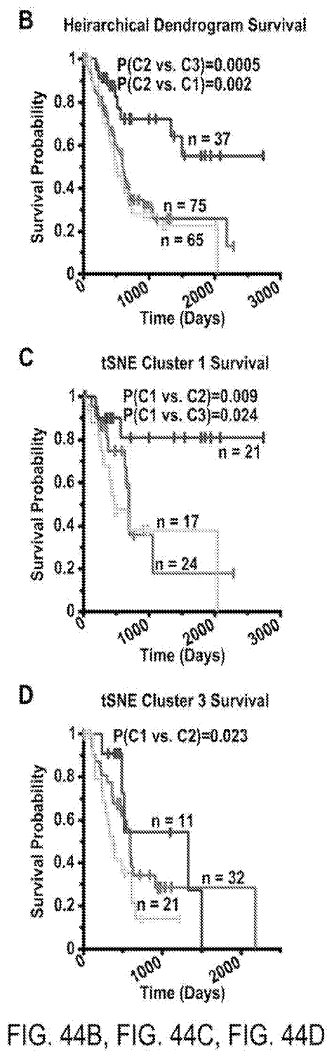

Whole transcriptome profiling can molecularly classify tumors and predict survival and therapeutic responses. To determine whether t-SNE can also be employed to refine survival predictions based on this approach or vice versa, RNAseq data was retrieved from several tumor types, generated heat maps of protein-coding transcripts and sub-classified tumors using hierarchical clustering. Initial focus was on pancreatic ductal adenocarcinoma because t-SNE analysis with Purine Biosynthesis Pathway transcripts identified 3 t-SNE clusters with borderline significant survival differences (P=0.048, FIGS. 6 and 19) and because the large cohort size permitted robust subsequent t-SNE analyses on each sub-population. Hierarchical clustering identified 3 molecular subgroups (FIG. 44A), 2 of which, dendrograms 1 and 3 (Dendro 1 and Dendro 3), were associated with inferior survival (FIG. 44B). Tumors from the 3 t-SNE clusters were about evenly distributed among these 3 Dendro groups (FIG. 44A). t-SNE Cluster 1 tumors can be further subdivided into two groups with significant differences in survival based upon their dendrogram identities (FIG. 44C). Similarly, t-SNE Cluster 2 tumors can also be divided into groups with significant differences in survival (FIG. 44D). Thus, t-SNE clusters, already predictive of survival, can be further stratified based on hierarchical clustering. Similarly, dendrogram groups contained patients whose survival can be further stratified based on t-SNE profiles.

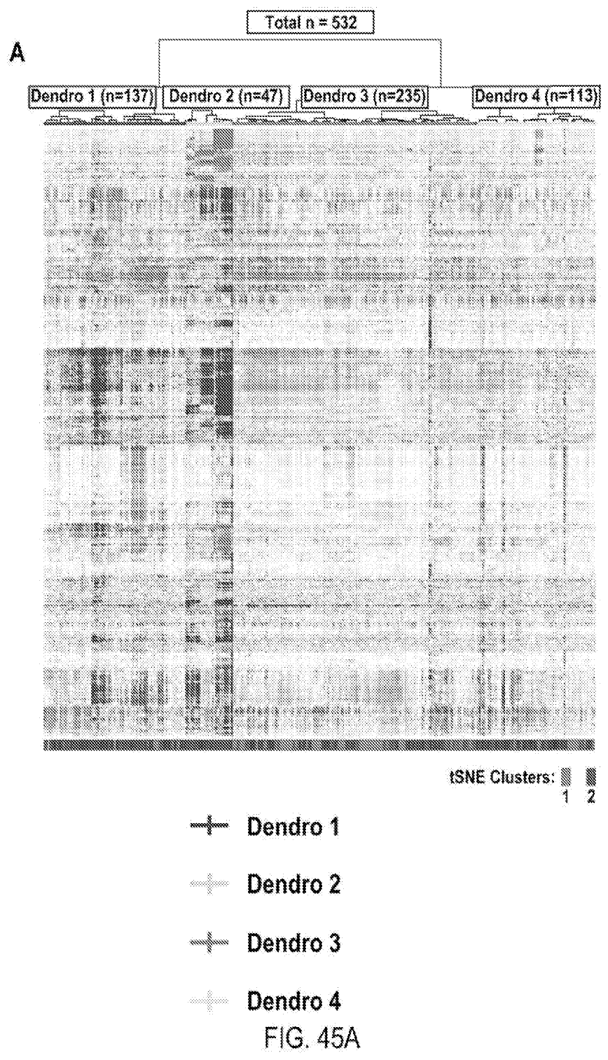

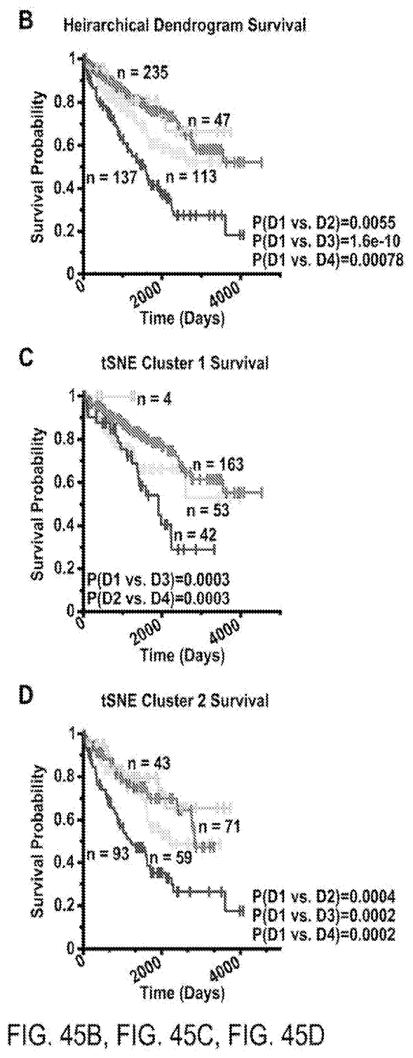

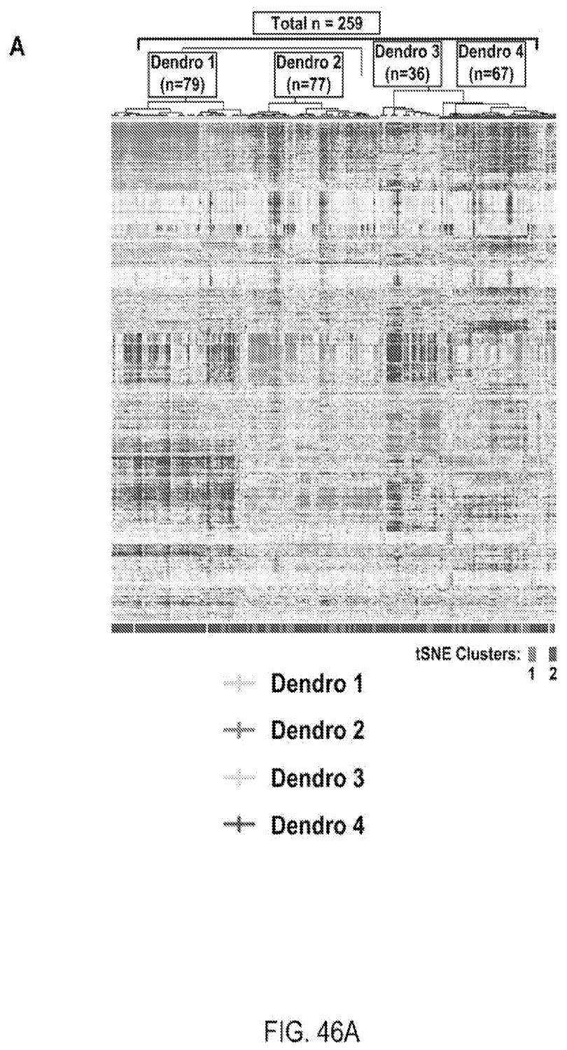

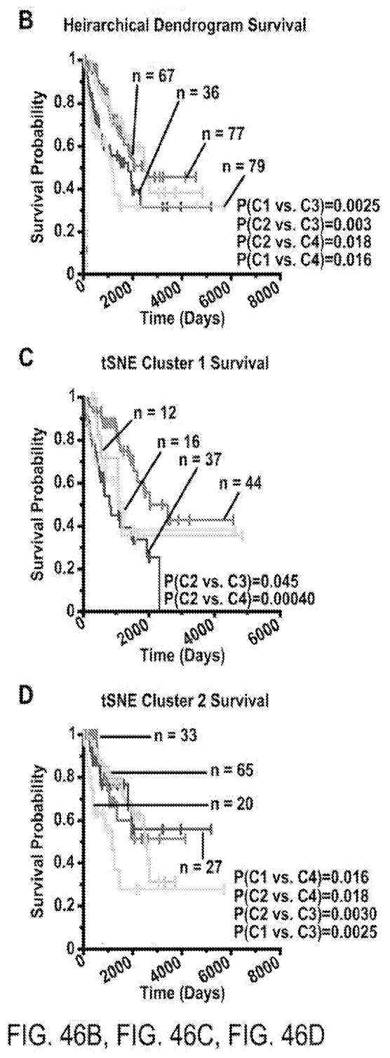

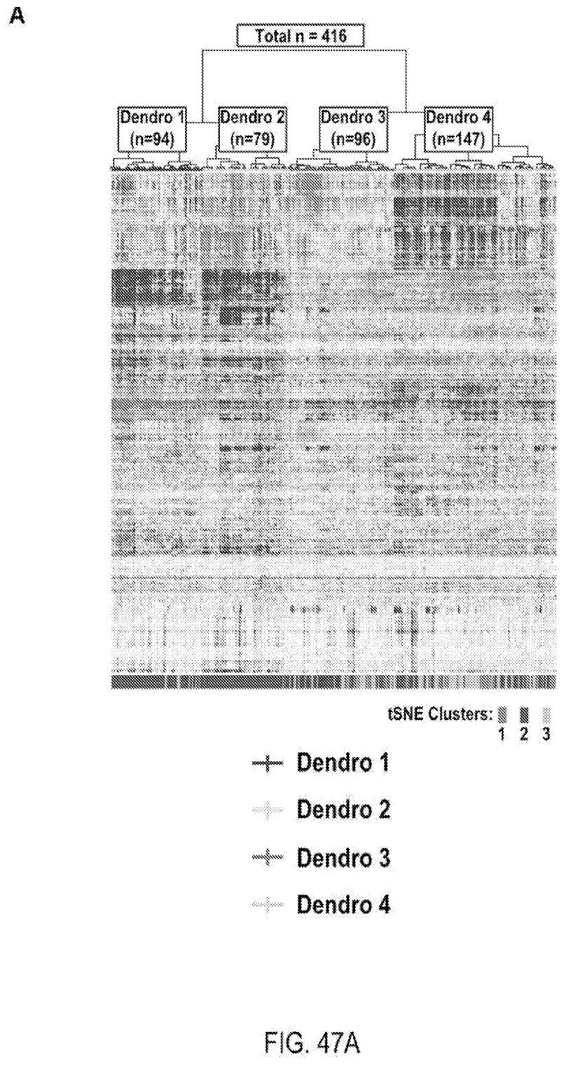

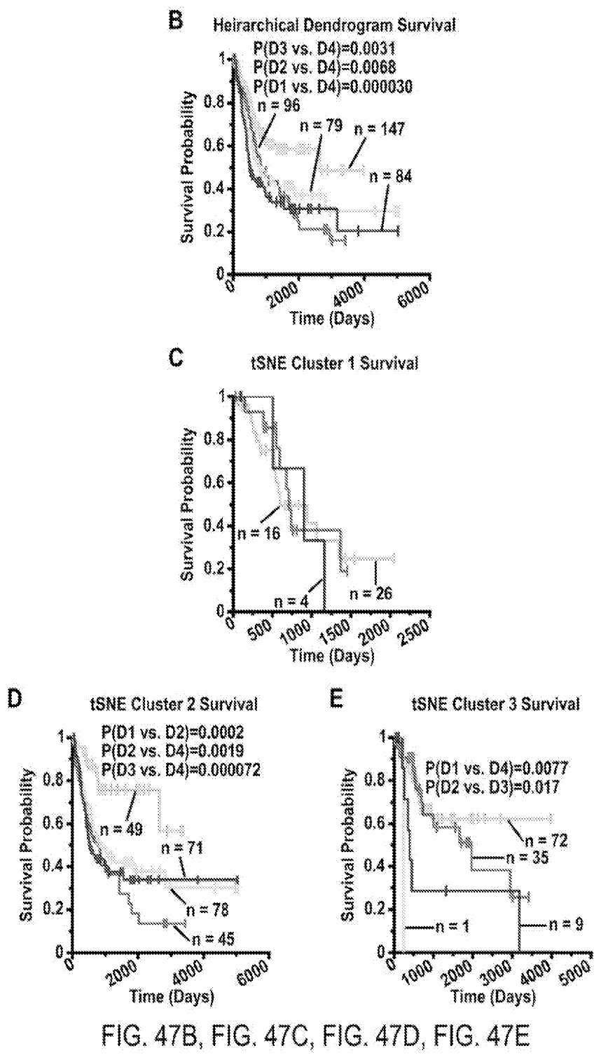

Different but related findings were made in clear cell kidney cancer, where whole transcriptome profiling generated 4 dendrograms (Dendrol-4) with Dendro 1 having particularly unfavorable survival (FIG. 45A & 45B). Unlike the more random distribution of t-SNE clusters seen in FIG. 44A, Dendro 1 group was overly populated by Pyrimidine Biosynthetic Pathway t-SNE Cluster 2 tumors (also with unfavorable outcomes) whereas the Dendro 3 group contained a greater preponderance of t-SNE 1 tumors with more favorable outcomes. Both t-SNE groups can be further sub-divided into distinct survival cohorts when further categorized by their respective dendro group (FIGS. 45C and 45D). Additional variations of these general themes were seen with Myc Pathway transcripts in sarcomas and TCA Cycle Pathway transcripts in Bladder Cancer (FIGS. 46 and 47). t-SNE-based analysis is thus comparable and in some cases even superior to whole transcriptome profiling for forecasting long-term survival. However, depending upon the tumor type under study, the two methods can be used in tandem to better define tumor subgroups with significantly different long-term survival patterns.

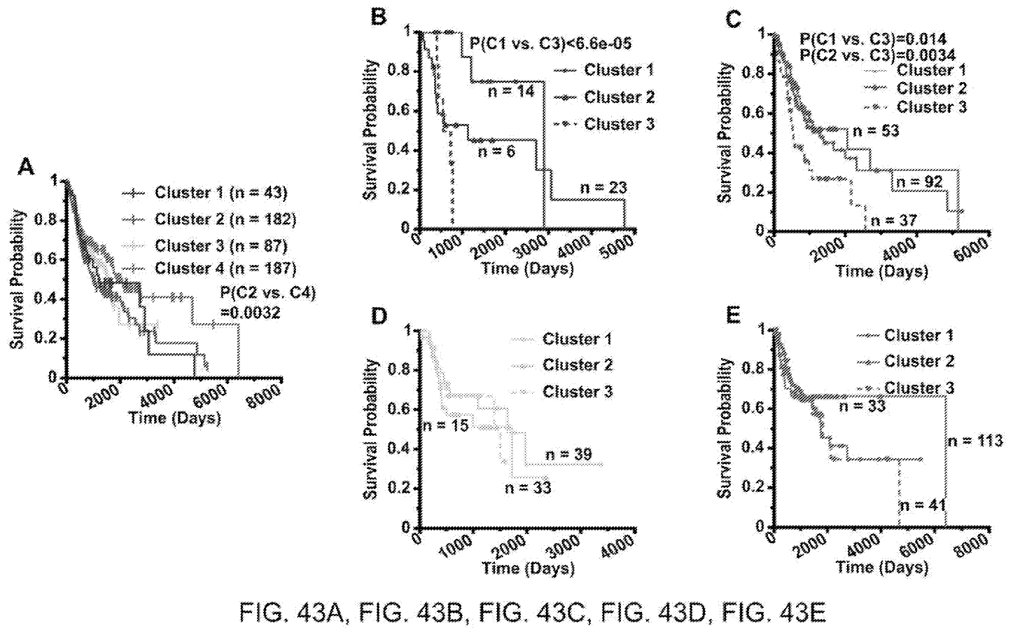

Together, these results show that t-SNE analysis of small numbers of CPTs from cancer-related pathways in tumors is comparable—or in some cases—even superior to genome-wide transcriptional profiling for predicting long-term survival. However, the addition of whole transcriptome profiling can further refine and/or confirm the prognostic value of t-SNE-based analyses. Conversely, the survival of specific Dendro groups, derived from the expression levels of several thousand transcripts, could in some cases be explained by their being heavily weighted with tumors bearing a specific t-SNE profile determined by the expression pattern of as few as 13 transcripts (FIG. 43).

(3) t-SNE Compliments Sub-Classification and Clinical Staging for Certain Cancers

Triple-negative breast cancer (TNBC), which represents 10-20% of all tumors, is defined by the lack of immuno-histochemical staining for the estrogen and progesterone receptors and the cell surface epidermal growth factor receptor HER2. It has the most unfavorable outcome of all breast cancer subtypes due primarily to its propensity for early metastatic recurrence. In contrast, the Luminal A form, representing 50-60% of all cases, has the most favorable long-term survival. Belying the apparent simplicity of this long-standing classification scheme, however, is the fact that TNBC and Luminal A variants have each been recently sub-classified into several distinct molecular entities based on whole transcriptomic profiling.

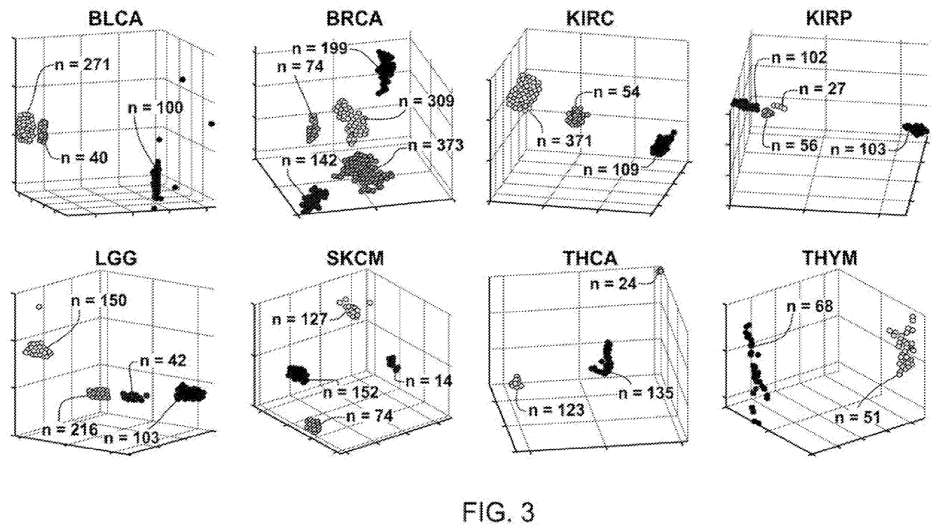

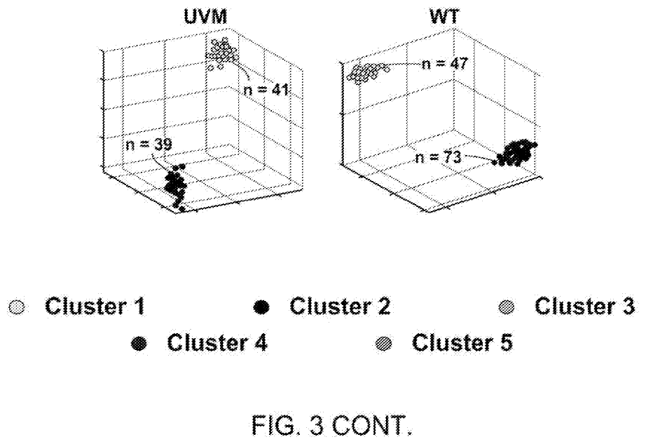

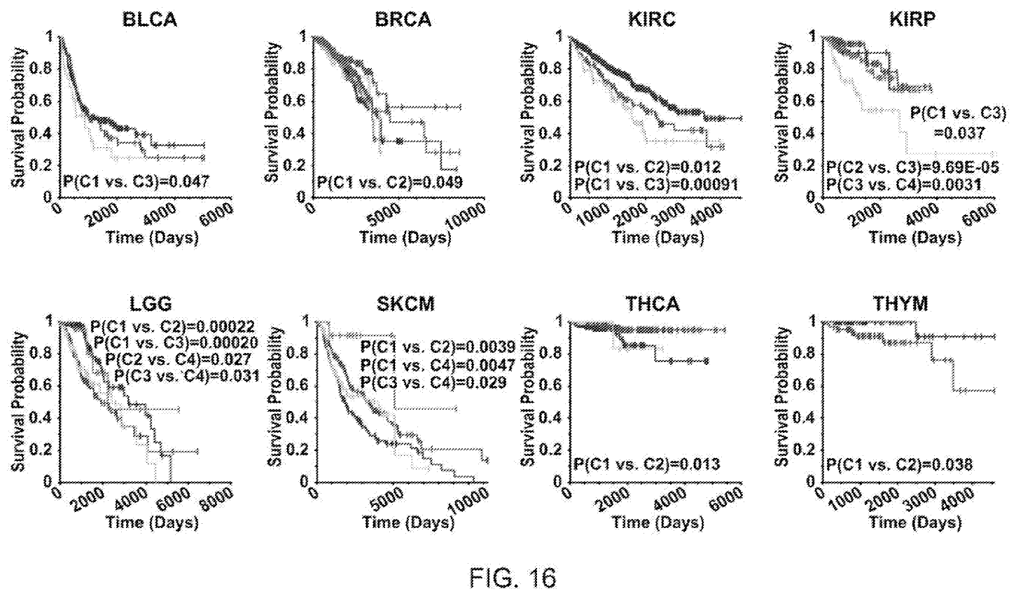

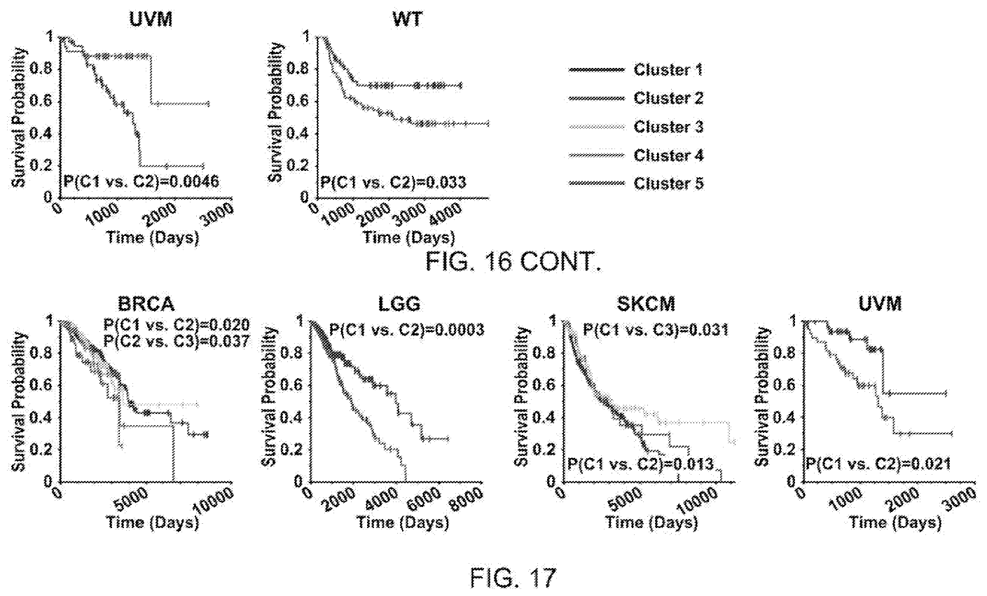

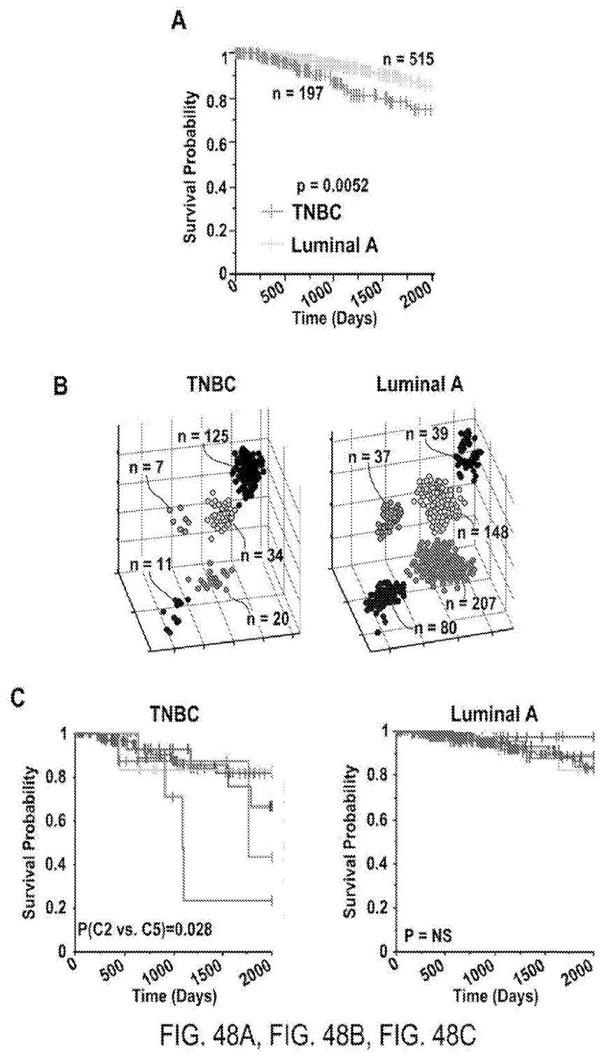

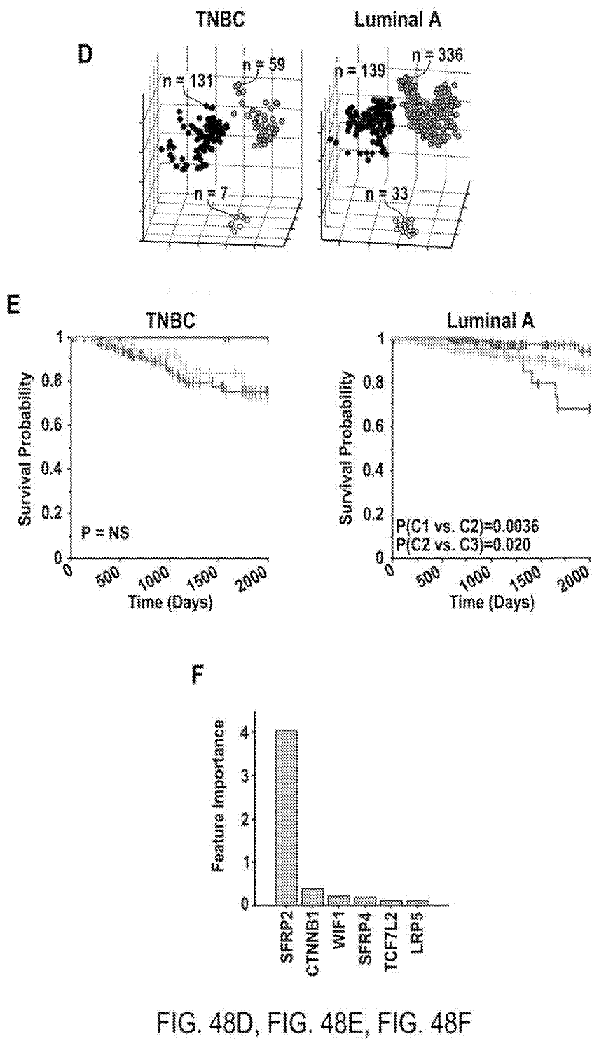

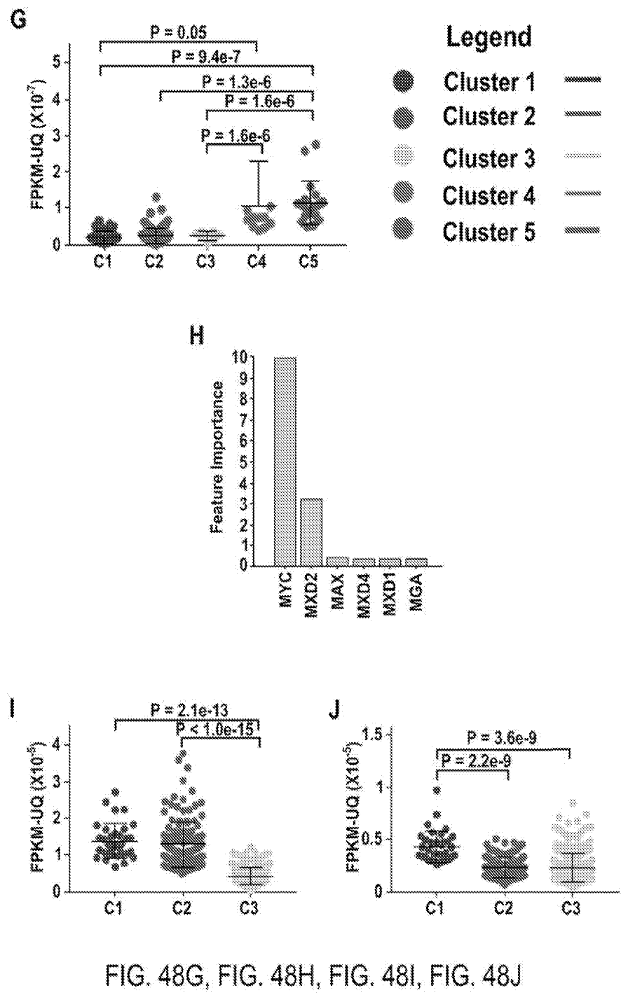

To determine whether t-SNE-based analyses could aid in refining the survival prediction for these two forms of breast cancer, we first confirmed these differences using data from the TCGA database (FIG. 48A). Because Wnt Pathway transcript t-SNE patterns had been predictive of survival in all breast cancer patients (FIG. 27, and FIGS. 3 and 16), we applied these analyses to the individual TNBC and Luminal A subtype populations. TNBCs comprised 17.9% of all tumors (197 of 1097) and occupied the same original five t-SNE clusters as their non-TNBC counterparts (FIG. 48B). However, these tumors were disproportionately grouped into Cluster 2, which contained 62.8% of the total TNBC population (P=4.2×10-60 based on Fisher's exact test), with the remaining four clusters each containing 5.3-11%. Luminal A cancers (46.5% of all tumors) were evenly distributed among t-SNE clusters 1,3,4 and 5 (48-56.3%) but were relatively depleted from Cluster 2 (19.5%. P=4.37×10-18). Thus, Cluster 2 was disproportionately comprised of a relative excess of TNBCs and a paucity of luminal A cancers. As a group, this Cluster's survival was identical to that of Clusters 1,3 and 4 whereas the smaller number of TNBCs within Cluster 5 (20/197=10.1%) was associated with a significantly worse long-term survival (FIG. 48C). Wnt pathway transcript patterns were not predictive of survival for luminal A cancers.

t-SNE-based profiling of breast cancers with Myc Pathway member transcripts did not initially identify groups with significantly different survival (FIG. 27). However, the analysis of Luminal A tumors but not TNBCs with this pathway's transcripts did further enhance survival prediction (FIGS. 48D and 48E). Taken together, these results demonstrate that, at least in the case of breast cancer, well-defined molecular subtypes could be further categorized by the subsequent interrogation with t-SNE-based transcriptional profiling.

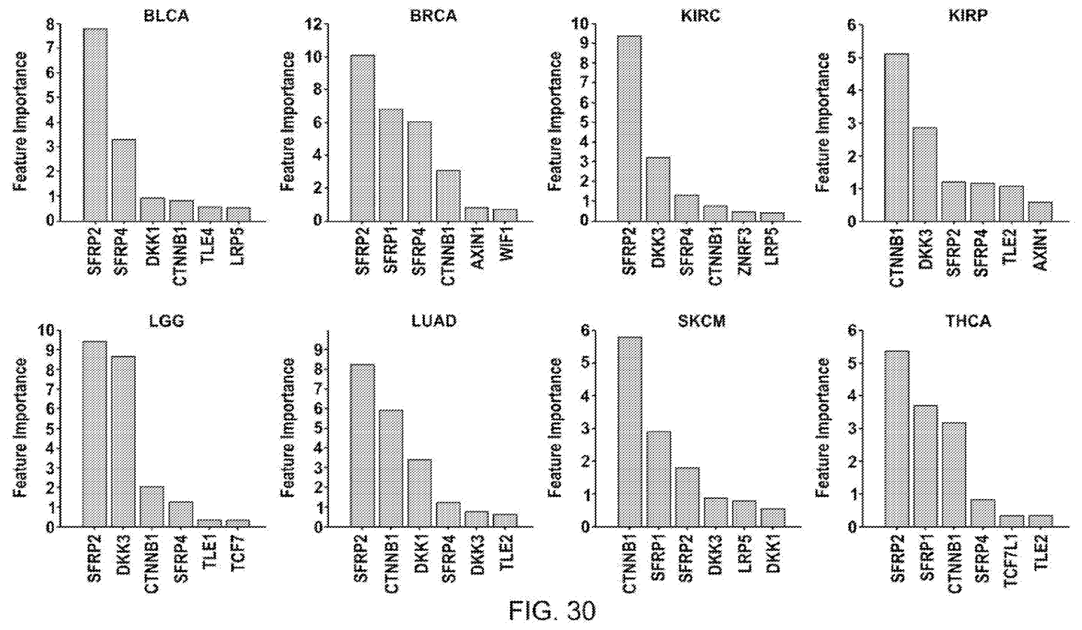

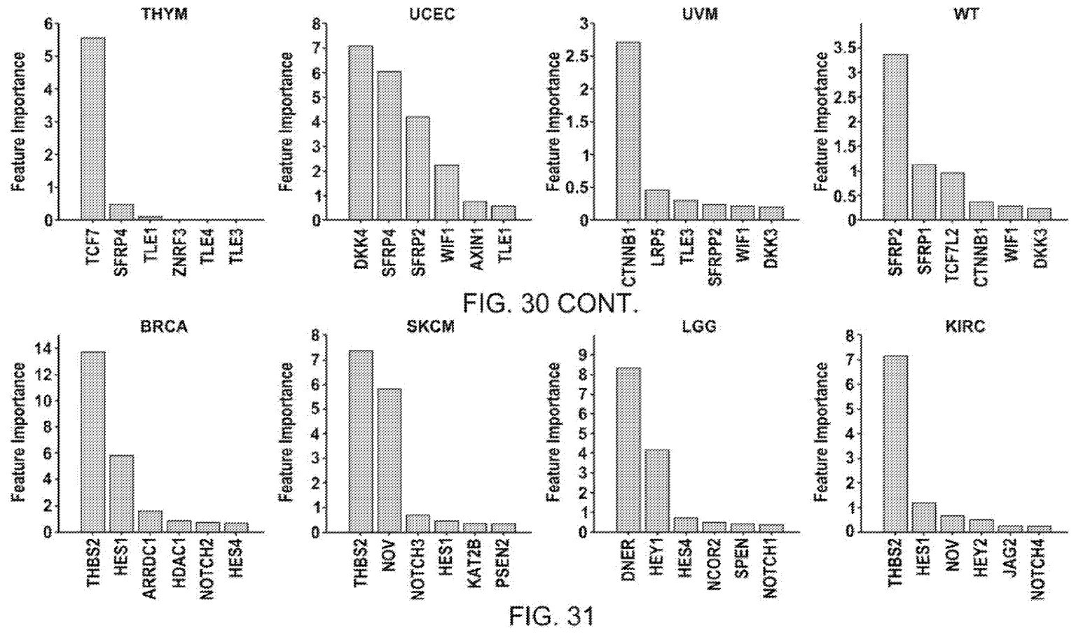

On average, Random Forest classification had shown that approximately three Wnt Pathway transcripts were the major determinants of t-SNE cluster profiles among the 12 different cancer types, including all breast cancers, where differential survival among Clusters was observed (FIG. 27). The most prominent of these transcripts were Sfrp2, Ctnnbl and Dkk1/3 (Feature Importance >1, FIG. 30). In the case of TNBC, however, this patterning was determined exclusively by Sfrp2 (FIG. 48F). Consistent with this, Cluster 5 tumors expressed the highest levels of Sfrp2 transcripts (FIG. 48G).

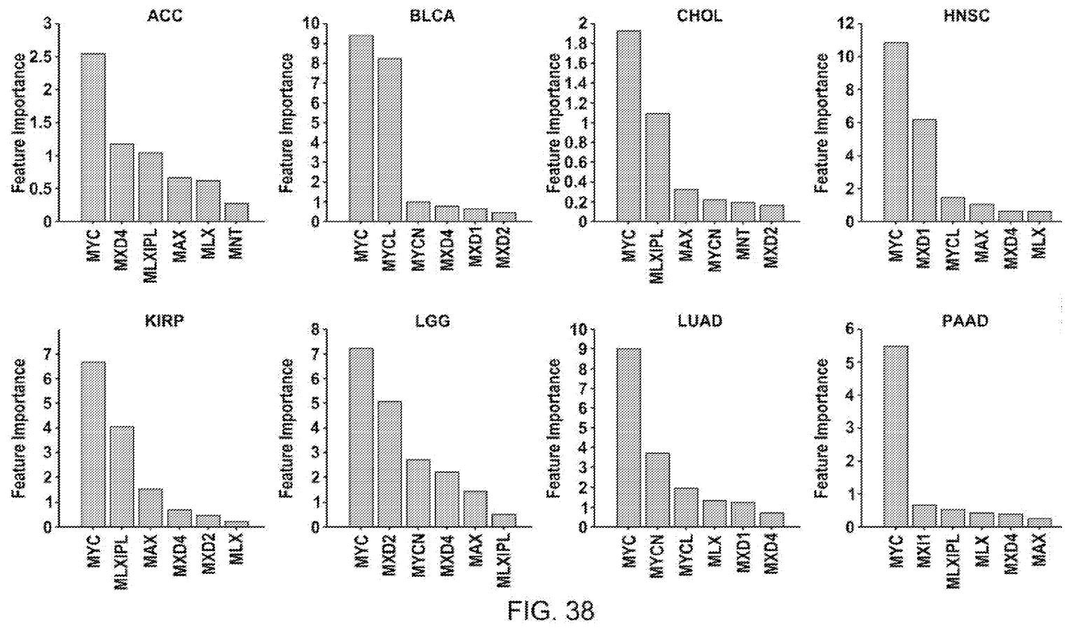

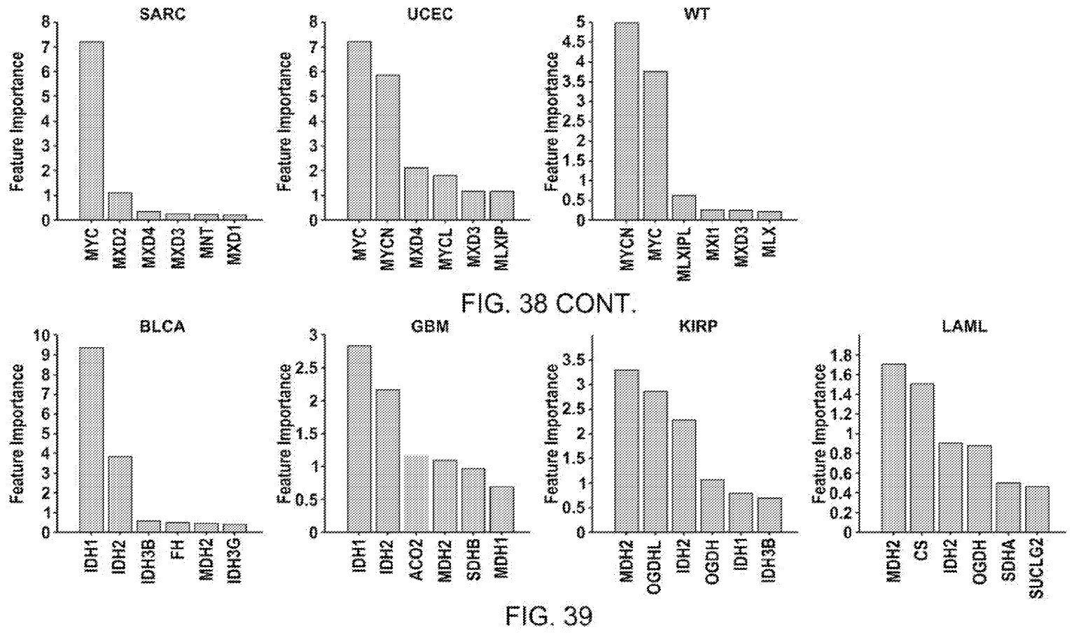

t-SNE clusters generated by Myc Pathway transcripts in 11 relevant tumor types were also determined by an average of three transcripts/tumor type with the most common ones being Myc, N-Myc and Mxd2 (FIG. 38). The t-SNE clusters of Luminal A cancers, in contrast, were more driven by Myc and Mxd2 (FIG. 48H). Interestingly, the Cluster 1 tumors of this subset, which expressed high levels of Myc and Mxd2 were associated with the worst prognosis (FIGS. 48I and 48J).

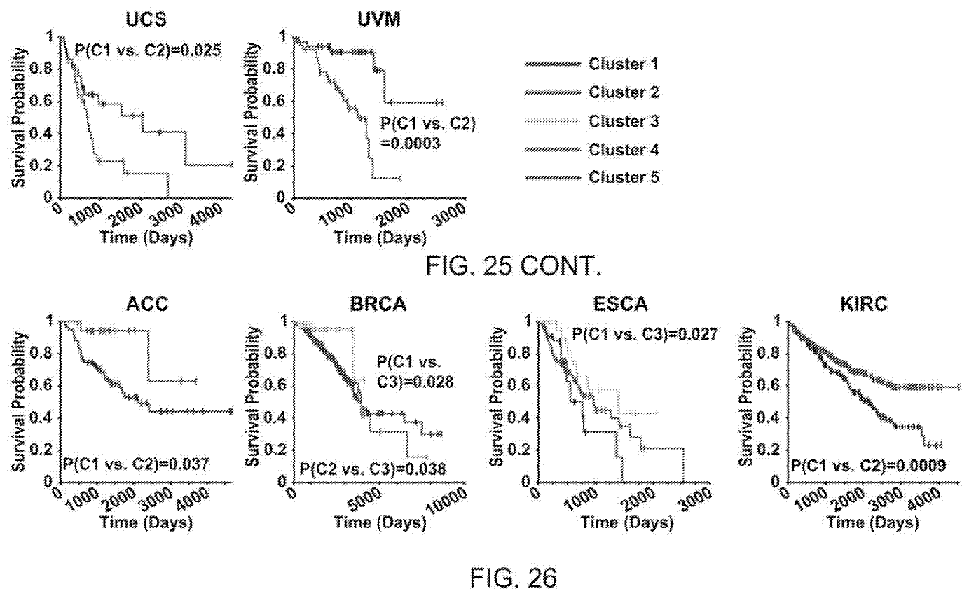

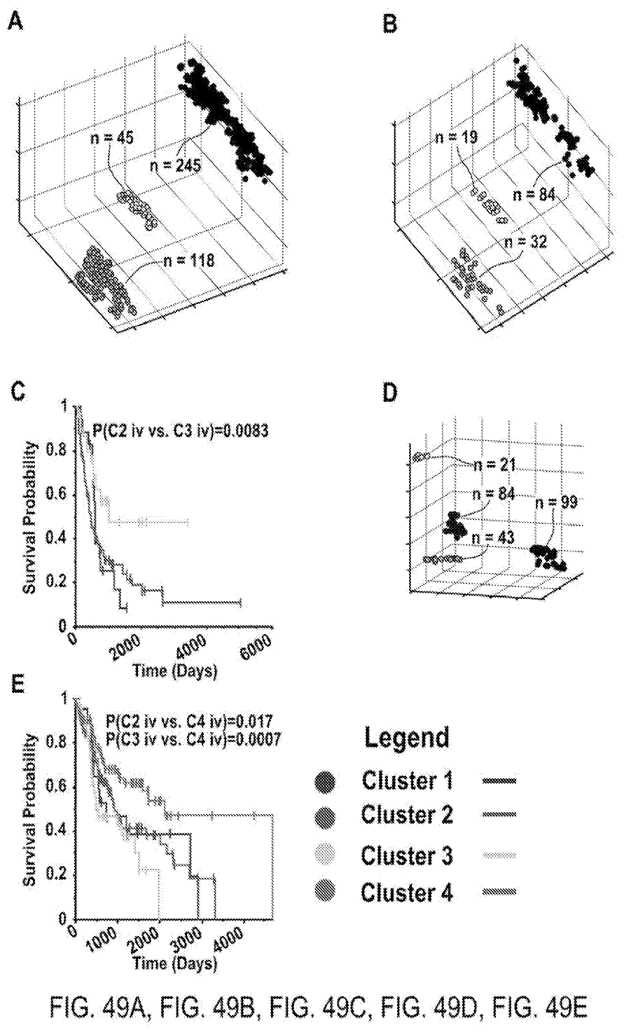

Lastly, we asked whether the survival of patients with advanced stage disease at the time of diagnosis could also be better stratified by t-SNE analysis. To this end, we re-analyzed the bladder cancers in TCGA (Table 2), 135 of which originated from patients with Stage IV disease. A Chi-square test indicated that the tumors were randomly distributed among the three previously identified t-SNE clusters ((P=0.073), FIG. 49A, 49B and FIG. 12). Just as t-SNE profiling had previously predicted differential survival in all patients with bladder cancer (FIG. 25), so too was it predictive of survival in individuals with Stage IV tumors with Cluster 3 tumors being associated with significantly more favorable survival (FIG. 49C).

Similar findings were made in head and neck squamous cell cancers where t-SNE profiling with Myc Pathway transcripts had previously identified four distinct clusters with significant survival differences (FIGS. 1 and 14). As with bladder cancers, the primary tumors from 247 Stage IV cancers were randomly distributed among these groups (P=0.075, FIG. 49D). Among these tumors, however, t-SNE Cluster 4 was associated with a significantly longer median survival (2120 days) than the other clusters (combined median survival=915 days).

c) Discussion

Herein is shown the feasibility of predicting survival in multiple cancer types based on the expression of small subsets of a 212 member cancer pathway transcript (CPT) collection. These originated from 12 canonical cancer pathways with well-established roles in cancer cell proliferation, survival and metabolism. However, unlike whole transcriptome analyses where expression levels correlate with survival in specific cancers (FIG. 44A, FIGS. 45, 46, and 47), the value of the analyses reported here lies in the t-SNE-generated expression patterns of small numbers of CPTs across multiple tumor types. Indeed, in 30 of 34 cancers, these patterns were so highly predictive of survival that transcripts from a single pathway sufficed for this purpose. Examples include the Cell Cycle Pathway (15 transcripts) in AML, the PI3K Pathway (18 members) in low-grade gliomas and any one of 9 pathways, each comprised of 6-30 transcripts, in clear cell kidney cancer (FIG. 27). Moreover, of the 30 cancer types for which t-SNE profiling was useful, an average of 3.7 pathways/tumor type correlated with survival, thus proving of predictive value in 91.4% of all cancers examined. This of course must be considered as provisional for other data bases given that the TCGA database may be biased toward particular cancer types. As other pathways' transcripts are added to the 12 reported here, it seems likely that they will prove valuable in the four cancer types for which the current collection is unhelpful.

Many of above pathways' transcripts encode oncoproteins and tumor suppressors such as MYCC, PTEN, TP53, and IDH1/2 whose mutation and/or de-regulation frequently correlate with various cancers and outcomes (Table 1). However, it is shown herein that an additional and more powerful prognostic aspect of these transcripts resides in the patterns they assume relative to other transcripts in the same pathway. These patterns likely serve as reporters for the unique transcriptional and post-transcriptional environments that characterize each cancer type and dictate its relevant behaviors in much the same way as does whole transcriptome hierarchical clustering. Such patterns are undoubtedly determined by numerous interdependent factors including chromatin conformation; the binding and activities of promoter-proximal complexes such as RNA polymerase II and Mediator; the number and binding affinities of adjacent transcriptional factor binding sites; the long-range contribution of protein-bound enhancers and super-enhancers and the regulation of all these by post-translational modifications, metabolites and additional tissue-specific proteins. Differences in mRNA splicing and stability further influence mature transcript expression levels in tissue- and tumor-specific ways. Based on presumably similar regulatory dependencies, other as yet unexamined pathways' t-SNE patterns will also likely correlate with survival and perhaps other aspects of tumor behavior such as therapeutic susceptibility and metastatic proclivity. It is also important to emphasize that the entire 212 transcript repertoire reported here is unnecessary for assessing any particular tumor type. Rather, particular pathways and subsets of transcripts within them can be selected based on those whose transcript t-SNE patterns are predictive for particular tumor types and transcript subsets that make disproportionate contributions to expression patterns (FIGS. 29, 30, 31, 32, 33, 34, 35, 36, 37, 38, 39, and 40). In the case of low-grade gliomas and clear cell renal cancer, this could be as many as 9 distinct pathways or as few as a single one for colo-rectal and prostate cancers (FIG. 27).

In some cases, additional prognostic information was extracted using sequential t-SNE analysis or whole transcriptome profiling (FIGS. 28 and 44 and FIGS. 41, 42, 43, 45, 46, and 47). Similarly, patient survival within individual whole-transcriptome hierarchical groups could in some cases be further refined by t-SNE. It is in tumor types such as pancreatic ductal adenocarcinoma where particular t-SNE profiles are more evenly distributed across the entire transcriptome spectrum that the combined advantages of these two independent approaches are likely to have the greatest impact (FIG. 44). Future efforts should focus on the additive benefit of such combinatorial analyses. The immediate prognostic advantage of these sequential approaches is currently likely to be limited in its statistical power by relatively small patient numbers.

TABLE 1
Component Transcripts and NCBI Gene ID Numbers Used for t-
SNE Profiling in Each of Twelve Cancer-Related Pathways.

| Pathway/Gene family | Gene Name | NCBI gene ID |
|---|---|---|
| Cell cycle | RB1 | 5925 |
| (15 members) | CDKN2C | 1031 |
| | CDKN2B | 1030 |
| | CDKN2A | 1029 |
| | CDKN1B | 1027 |
| | CDKN1A | 1026 |
| | E2F3 | 1871 |
| | E2F1 | 1869 |
| | CDK6 | 1021 |
| | CDK4 | 1019 |
| | CDK2 | 1017 |
| | CCNE1 | 898 |
| | CCND3 | 896 |
| | CCND2 | 894 |
| | CCND1 | 595 |
| Wnt/β-Catenin | ZNRF3 | 84133 |
| (25 members) | WIF1 | 11197 |
| | TLE4 | 7091 |
| | TLE3 | 7090 |
| | TLE2 | 7089 |
| | TLE1 | 7088 |
| | TCF7L2 | 6934 |
| | TCF7L1 | 83439 |
| | TCF7 | 6932 |
| | SFRP5 | 6425 |
| | SFRP4 | 6424 |
| | SFRP2 | 6423 |
| | SFRP1 | 6422 |
| | RNF43 | 54894 |
| | LRP5 | 4041 |
| | GSK3B | 2932 |
| | DKK4 | 27121 |
| | DKK3 | 27122 |
| | DKK2 | 27123 |
| | DKK1 | 22943 |
| | CTNNB1 | 1499 |
| | AXIN2 | 8313 |
| | AXIN1 | 8312 |
| | APC | 324 |
| | AMER1 | 139285 |
| TP53 | CHEK2 | 11200 |
| (6 members) | ATM | 472 |
| | TP53 | 7157 |
| | RPS6KA3 | 6197 |
| | MDM4 | 4194 |
| | MDM2 | 4193 |
| TGF-β | TGFBR1 | 7046 |
| (7 members) | TGFBR2 | 7048 |
| | ACVR2A | 92 |
| | ACVR1B | 91 |
| | SMAD2 | 4087 |
| | SMAD3 | 4088 |
| | SMAD4 | 4089 |
| Notch | ARRDC1 | 92714 |
| (30 members) | CNTN6 | 27255 |
| | CREBBP | 1387 |
| | EP300 | 2033 |
| | HES1 | 3280 |
| | HES2 | 54626 |
| | HES3 | 390992 |
| | HES4 | 57801 |
| | HES5 | 388585 |
| | HEY1 | 23462 |
| | HEY2 | 23493 |
| | HEYL | 26508 |
| | KAT2B | 8850 |
| | KDM5A | 5927 |
| | NOTCH1 | 4851 |
| | NOTCH2 | 4853 |
| | NOTCH3 | 4854 |
| | NOTCH4 | 4855 |
| | NOV | 4856 |
| | PSEN2 | 5664 |
| | SPEN | 23013 |
| | FBXW7 | 55294 |
| | THBS2 | 7058 |
| | CUL1 | 8454 |
| | NCOR1 | 9611 |
| | NCOR2 | 9612 |
| | HDAC1 | 3065 |
| | JAG2 | 3714 |
| | MAML3 | 55534 |
| | DNER | 92737 |
| PI3 Kinase | MTOR | 2475 |
| (18 members) | RICTOR | 253260 |
| | RPTOR | 57521 |
| | RHEB | 6009 |
| | TSC2 | 7249 |
| | TSC1 | 7248 |
| | PPP2R1A | 5518 |
| | AKT3 | 10000 |
| | AKT2 | 208 |
| | AKT1 | 207 |
| | STK11 | 6794 |
| | INPP4B | 8821 |
| | PIK3R3 | 8503 |
| | PIK3R2 | 5296 |
| | PIK3R1 | 5295 |
| | PTEN | 5728 |
| | PIK3CB | 5291 |
| | PIK3CA | 5290 |
| Hippo | YAP1 | 10413 |
| (27 members) | WWTR1 | 25937 |
| | TEAD2 | 8463 |
| | STK4 | 6789 |
| | STK3 | 6788 |
| | SAV1 | 60485 |
| | LATS1 | 9113 |
| | LATS2 | 26524 |
| | MOB1A | 55233 |
| | MOB1B | 92597 |
| | PTPN14 | 5784 |
| | NF2 | 4771 |
| | WWC1 | 23286 |
| | TAOK1 | 57551 |
| | TAOK2 | 9344 |
| | TAOK3 | 51347 |
| | CRB1 | 23418 |
| | CRB2 | 286204 |
| | CRB3 | 92359 |
| | FAT1 | 2195 |
| | FAT2 | 2196 |
| | FAT3 | 120114 |
| | FAT4 | 79633 |
| | DCHS1 | 8642 |
| | DCHS2 | 54798 |
| | CSNK1E | 1454 |
| | CSNK1D | 1453 |
| Myc | MYC | 4609 |
| (13 members) | MXI1 | 4601 |
| | MYCL | 4610 |
| | MYCN | 4613 |
| | MAX | 4149 |
| | MXD1 | 4084 |
| | MXD3 | 83463 |
| | MXD4 | 10608 |
| | MLX | 6945 |
| | MLXIPL | 51085 |
| | MLXIP | 22877 |
| | MNT | 4335 |
| | MGA | 23269 |
| Purine | PPAT | 5471 |
| Biosynthesis | GART | 2618 |
| (25 members) | PFAS | 5198 |
| | PAICS | 10606 |
| | ADSL | 158 |
| | ATIC | 471 |
| | ADSSL1 | 122622 |
| | ADSS | 159 |
| | AK1 | 203 |
| | AK2 | 204 |
| | AK3 | 50808 |
| | AK4 | 205 |
| | AK5 | 26289 |
| | AK7 | 122481 |
| | RRM1 | 6240 |
| | RRM2 | 6241 |
| | GMPS | 8833 |
| | GUK1 | 2987 |
| | NME1 | 4830 |
| | NME2 | 4831 |
| | NME3 | 4832 |
| | NME4 | 4833 |
| | NME5 | 8382 |
| | NME6 | 10201 |
| | NME7 | 29922 |
| Pyrimidine | CAD | 790 |
| Biosynthesis | DHODH | 1723 |
| (23 members) | UMPS | 7372 |
| | CMPK1 | 51727 |
| | CMPK2 | 129607 |
| | NME1 | 4830 |
| | NME2 | 4831 |
| | NME3 | 4832 |
| | NME4 | 4833 |
| | NME5 | 8382 |
| | NME6 | 10201 |
| | NME7 | 29922 |
| | CTPS1 | 1503 |
| | CTPS2 | 56474 |
| | RRM1 | 6240 |
| | RRM2 | 6241 |
| | DUT | 1854 |
| | ENPP3 | 5169 |
| | ENPP1 | 5167 |
| | ITPA | 3704 |
| | TYMS | 7298 |
| | DTYMK | 1841 |
| | NTPCR | 84284 |
| TCA Cycle | OGDH | 4967 |
| (21 members) | OGDHL | 55753 |
| | CS | 1431 |
| | ACO1 | 48 |
| | ACO2 | 50 |
| | IDH1 | 3417 |
| | IDH2 | 3418 |
| | IDH3A | 3419 |
| | IDH3B | 3420 |
| | IDH3G | 3421 |
| | SUCLA2 | 8803 |
| | SUCLG1 | 8802 |
| | SUCLG2 | 8801 |
| | SDHA | 6389 |
| | SDHB | 6390 |
| | SDHC | 6391 |
| | SDHD | 6392 |
| | FH | 2271 |
| | MDH1 | 4190 |
| | MDH1B | 130752 |
| | MDH2 | 4191 |
| Pentose | H6PD | 9563 |
| phosphate | PGLS | 25796 |
| pathway | G6PD | 2539 |
| (11 members) | RPIA | 22934 |
| | PGD | 5226 |
| | RPE | 6120 |
| | RPEL1 | 729020 |
| | TALDO1 | 6888 |
| | TKT | 7086 |
| | TKTL1 | 8277 |
| | TKTL2 | 84076 |

A total of 221 transcripts are listed but 9 of those in the Purine and Pyrimidine Biosynthesis Pathways (depicted in red) are common. Thus, a total of 212 unique transcripts were used for generating t-SNE profiles.

TABLE 2
Abbreviations for and Number of Cancers
in Each of the TCGA Groups

| Abbre- | | Number of |
|---|---|---|
| viation | Cancer Type | Tumors |
| AML | Acute myelogenous (bone marrow) | 119 |
| ACC | Adrenocortical carcinoma | 79 |
| BLCA | Bladder urothelial carcinoma | 411 |
| BLGG | Brain: low-grade glioma | 511 |
| BRIC | Invasive breast cancer | 1097 |
| CESC | Cervical/endocervical squamous cell | 304 |
| | carcinoma | |
| CHOL | Cholangiocarcinoma | 36 |
| COAD | Colon adenocarcinoma | 469 |
| DLBC | Diffuse large B-cell lymphoma | 48 |
| ESCA | Esophageal carcinoma | 161 |
| GBM | Glioblastoma multiforme | 155 |
| HNSC | Head & neck squamous cell carcinoma | 500 |
| HRWT | High-risk Wilms' tumor | 120 |
| KICH | Kidney chromophobe carcinoma | 65 |
| KIRC | Kidney clear cell carcinoma | 534 |
| KIRP | Kidney papillary carcinoma | 288 |
| LIHC | Hepatocellular carcinoma | 371 |
| LUAD | Lung adenocarcinoma | 524 |
| LUSC | Lung squamous cell carcinoma | 501 |
| MESO | Mesothelioma | 86 |
| OV | Ovarian serous cystadenocarcinoma | 374 |
| PAAD | Pancreatic adenocarcinoma | 177 |
| PCPG | Pheochromocytoma/paraganglioneuroma | 178 |
| PRAD | Prostate adenocarcinoma | 498 |
| READ | Rectal adenocarcinoma | 166 |
| SARC | Sarcoma | 259 |
| SKCM | Metastatic cutaneous melanoma | 367 |
| STAD | Stomach (gastric) adenocarcinoma | 375 |
| TGCT | Testicular germ cell tumor | 150 |
| THCA | Thyroid carcinoma | 502 |
| THYM | Thymoma | 119 |
| UCS | Uterine carcinosarcoma | 56 |
| UCEC | Uterine corpus endometrial carcinoma | 547 |
| UVM | Uveal melanoma | 80 |

TABLE 3
t-SNE clustering parameters.

| | | | Learning | Covariance | Shared | Perturb | Perturb |
|---|---|---|---|---|---|---|---|
| Pathway | Cancer | Perplexity | Rate | Type | Covariance | Input | Output |
| TCA | LAML | 6 | 1 | Full | TRUE | FALSE | FALSE |
| TCA | BLCA | 12 | 10 | Full | TRUE | FALSE | FALSE |
| TCA | GBM | 6 | 10 | Diagonal | TRUE | FALSE | FALSE |
| TCA | KIRP | 6 | 10 | Full | TRUE | TRUE | FALSE |
| TCA | PRAD | 11 | 10 | Diagonal | FALSE | TRUE | FALSE |
| TCA | READ | 8 | 10 | Diagonal | FALSE | TRUE | FALSE |
| TCA | UCS | 6 | 10 | Full | TRUE | FALSE | FALSE |
| TCA | UVM | 5 | 1 | Diagonal | FALSE | TRUE | FALSE |
| Purine | LAML | 5 | 10 | Full | TRUE | FALSE | FALSE |
| Purine | BRCA | 18 | 100 | Full | TRUE | FALSE | FALSE |
| Purine | CESC | 9 | 100 | Diagonal | TRUE | FALSE | FALSE |
| Purine | HRWT | 5 | 10 | Full | FALSE | FALSE | FALSE |
| Purine | KIRC | 11 | 100 | Full | FALSE | TRUE | FALSE |
| Purine | LIHC | 7 | 10 | Full | TRUE | TRUE | TRUE |
| Purine | LUAD | 9 | 100 | Full | TRUE | FALSE | FALSE |
| Purine | MESO | 11 | 1 | Full | TRUE | FALSE | FALSE |
| Purine | PAAD | 8 | 10 | Full | TRUE | FALSE | FALSE |
| Purine | SARC | 7 | 100 | Diagonal | FALSE | TRUE | FALSE |
| Purine | UCEC | 10 | 10 | Full | FALSE | FALSE | FALSE |
| Purine | UVM | 8 | 10 | Diagonal | FALSE | TRUE | FALSE |
| Pyrimidine | ACC | 7 | 10 | Full | FALSE | FALSE | FALSE |
| Pyrimidine | LGG | 11 | 10 | Diagonal | FALSE | TRUE | FALSE |
| Pyrimidine | BRCA | 17 | 100 | Diagonal | TRUE | FALSE | FALSE |
| Pyrimidine | KICH | 5 | 10 | Full | TRUE | FALSE | FALSE |
| Pyrimidine | KIRC | 12 | 10 | Full | TRUE | FALSE | FALSE |
| Pyrimidine | LIHC | 10 | 10 | Diagonal | FALSE | TRUE | FALSE |
| Pyrimidine | OV | 10 | 10 | Full | TRUE | FALSE | FALSE |
| Pyrimidine | THYM | 11 | 10 | Diagonal | FALSE | TRUE | FALSE |
| Pyrimidine | UCEC | 10 | 10 | Diagonal | FALSE | TRUE | FALSE |
| Cell Cycle | LAML | 7 | 1 | Diagonal | FALSE | TRUE | FALSE |
| Cell Cycle | CESC | 12 | 10 | Full | TRUE | TRUE | FALSE |
| Cell Cycle | HNSC | 13 | 100 | Full | TRUE | FALSE | FALSE |
| Cell Cycle | KICH | 5 | 1 | Diagonal | FALSE | TRUE | FALSE |
| Cell Cycle | KIRC | 17 | 100 | Full | TRUE | FALSE | FALSE |
| Cell Cycle | KIRP | 8 | 10 | Diagonal | FALSE | TRUE | FALSE |
| Cell Cycle | LIHC | 13 | 100 | Diagonal | FALSE | FALSE | TRUE |
| Cell Cycle | MESO | 5 | 1 | Diagonal | FALSE | TRUE | FALSE |
| Cell Cycle | OV | 12 | 10 | Full | TRUE | TRUE | FALSE |
| Cell Cycle | PAAD | 5 | 100 | Diagonal | FALSE | TRUE | FALSE |
| Cell Cycle | SKCM | 9 | 100 | Diagonal | FALSE | TRUE | FALSE |
| Cell Cycle | THYM | 9 | 100 | Full | TRUE | FALSE | FALSE |
| Cell Cycle | UCEC | 13 | 10 | Full | TRUE | FALSE | FALSE |
| Cell Cycle | UVM | 8 | 10 | Full | TRUE | FALSE | FALSE |
| Hippo | LAML | 7 | 1 | Full | FALSE | FALSE | FALSE |
| Hippo | LGG | 9 | 100 | Diagonal | FALSE | TRUE | TRUE |
| Hippo | CHOL | 5 | 10 | Diagonal | FALSE | TRUE | FALSE |
| Hippo | COAD | 10 | 10 | Diagonal | FALSE | TRUE | FALSE |
| Hippo | MESO | 5 | 1 | Diagonal | TRUE | FALSE | FALSE |
| Hippo | SKCM | 8 | 100 | Full | TRUE | FALSE | FALSE |
| Hippo | THYM | 5 | 10 | Full | TRUE | FALSE | FALSE |
| Myc | ACC | 5 | 1 | Full | FALSE | TRUE | FALSE |
| Myc | BLCA | 11 | 10 | Diagonal | FALSE | TRUE | FALSE |
| Myc | LGG | 10 | 1 | Diagonal | FALSE | FALSE | FALSE |
| Myc | CHOL | 5 | 1 | Diagonal | FALSE | TRUE | FALSE |
| Myc | HNSC | 9 | 10 | Full | TRUE | FALSE | FALSE |
| Myc | HRWT | 9 | 1 | Diagonal | FALSE | TRUE | FALSE |
| Myc | KIRP | 13 | 10 | Full | TRUE | FALSE | FALSE |
| Myc | LUAD | 9 | 10 | Full | TRUE | FALSE | FALSE |
| Myc | PAAD | 9 | 10 | Full | TRUE | TRUE | TRUE |
| Myc | SARC | 10 | 10 | Diagonal | FALSE | TRUE | FALSE |
| Myc | UCEC | 11 | 100 | Full | TRUE | FALSE | FALSE |
| Notch | LGG | 18 | 10 | Diagonal | FALSE | TRUE | FALSE |
| Notch | BRCA | 17 | 100 | Diagonal | FALSE | TRUE | TRUE |
| Notch | HRWT | 8 | 1 | Diagonal | FALSE | TRUE | FALSE |
| Notch | KIRC | 10 | 100 | Diagonal | FALSE | TRUE | FALSE |
| Notch | MESO | 5 | 10 | Diagonal | FALSE | FALSE | TRUE |
| Notch | SKCM | 11 | 10 | Full | TRUE | FALSE | FALSE |
| Notch | UVM | 8 | 10 | Full | TRUE | FALSE | FALSE |
| Pentose Phosphate | ACC | 5 | 10 | Diagonal | FALSE | FALSE | FALSE |
| Pentose Phosphate | LGG | 11 | 100 | Diagonal | FALSE | FALSE | FALSE |
| Pentose Phosphate | BRCA | 9 | 100 | Diagonal | FALSE | FALSE | TRUE |
| Pentose Phosphate | ESCA | 7 | 10 | Diagonal | TRUE | FALSE | FALSE |
| Pentose Phosphate | KIRC | 11 | 100 | Diagonal | FALSE | TRUE | FALSE |
| Pentose Phosphate | KIRP | 10 | 100 | Full | TRUE | FALSE | FALSE |
| Pentose Phosphate | LIHC | 10 | 10 | Full | TRUE | FALSE | FALSE |
| Pentose Phosphate | MESO | 7 | 1 | Diagonal | TRUE | TRUE | FALSE |
| Pentose Phosphate | SARC | 9 | 10 | Diagonal | FALSE | FALSE | FALSE |
| Pentose Phosphate | THYM | 8 | 1 | Full | TRUE | FALSE | FALSE |
| Pentose Phosphate | UVM | 5 | 10 | Full | TRUE | FALSE | FALSE |
| PI 3-Kinase | LGG | 12 | 10 | Diagonal | FALSE | TRUE | FALSE |
| PI 3-Kinase | KIRC | 11 | 100 | Diagonal | FALSE | TRUE | FALSE |
| PI 3-Kinase | LIHC | 11 | 10 | Diagonal | TRUE | FALSE | FALSE |
| TGF-β | ACC | 7 | 10 | Full | TRUE | FALSE | FALSE |
| TGF-β | LGG | 11 | 10 | Diagonal | TRUE | FALSE | FALSE |
| TGF-β | ESCA | 9 | 1 | Diagonal | FALSE | FALSE | FALSE |
| TGF-β | HRWT | 9 | 100 | Diagonal | FALSE | TRUE | FALSE |
| TGF-β | KIRC | 12 | 10 | Full | TRUE | FALSE | FALSE |
| TGF-β | LIHC | 9 | 10 | Diagonal | FALSE | FALSE | FALSE |
| TGF-β | LUAD | 13 | 10 | Full | TRUE | FALSE | FALSE |
| TGF-β | SARC | 8 | 100 | Diagonal | FALSE | TRUE | FALSE |
| TP53 | ACC | 8 | 10 | Diagonal | FALSE | TRUE | FALSE |
| TP53 | LGG | 12 | 10 | Diagonal | FALSE | TRUE | FALSE |
| TP53 | GBM | 14 | 10 | Diagonal | FALSE | TRUE | FALSE |
| TP53 | KIRC | 12 | 10 | Full | FALSE | FALSE | FALSE |
| TP53 | STAD | 15 | 10 | Diagonal | FALSE | TRUE | FALSE |
| TP53 | UCS | 11 | 10 | Full | TRUE | FALSE | FALSE |
| Wnt | BLCA | 16 | 10 | Full | TRUE | FALSE | TRUE |
| Wnt | LGG | 16 | 10 | Full | TRUE | FALSE | TRUE |
| Wnt | BRCA | 21 | 10 | Diagonal | FALSE | TRUE | FALSE |
| Wnt | HRWT | 10 | 10 | Diagonal | FALSE | FALSE | TRUE |
| Wnt | KIRC | 12 | 10 | Full | TRUE | TRUE | TRUE |
| Wnt | KIRP | 13 | 100 | Full | TRUE | FALSE | FALSE |
| Wnt | LUAD | 15 | 10 | Full | TRUE | FALSE | FALSE |
| Wnt | SKCM | 16 | 10 | Diagonal | FALSE | FALSE | TRUE |
| Wnt | THYM | 10 | 10 | Diagonal | FALSE | TRUE | FALSE |
| Wnt | THCA | 18 | 100 | Diagonal | FALSE | TRUE | FALSE |
| Wnt | UCEC | 22 | 10 | Diagonal | FALSE | TRUE | FALSE |
| Wnt | UVM | 12 | 10 | Diagonal | FALSE | FALSE | TRUE |

Perplexity: the perplexity used for maximizing tSNE clusters for each cancer type. Learning Rate: The learning rate used for the tSNE. Covariance type: the type of covariance matrix used for fitting the GMM. For “Diagonal” covariance matrices, only the diagonal entries were non-zero, and the principle axes of the fitted Gaussians were parallel to the X,Y, and Z axes. For full covariance matrices, any entry could be non-zero, and the principle axes of the fitted Gaussians could be oriented in any direction. Shared Covariance: in cases where “TRUE”, each fitted Gaussian had the same covariance matrix. When “FALSE”, every fitted Gaussian had a unique covariance matrix. Perturb Input: where TRUE, the tSNE data were randomly perturbed by a maximum of 5% of the radius of the sphere enclosing all of the tSNE data prior to clustering. Perturb Output: where TRUE, the tSNE scatter-plots displayed in the figures have the aforementioned perturbation applied.

### B. REFERENCES

Audic Y, Hartley R S. Post-transcriptional regulation in cancer. Biol Cell. 2004; 96:479-98.
Bradner J E, Hnisz D, Young R A. Transcriptional Addiction in Cancer. Cell. 2017; 168:629-643.
Breiman, L. Random forests. Machine Learning. 2001; 45:5-32, 2001.
Broom B M, Ryan M C, Brown R E, et al. A galaxy implementation of next-generation clustered heatmaps for interactive exploration of molecular profiling data. Cancer Res. 2017; 77:e23-e26.
Buj R, Aird K M. Deoxyribonucleotide triphosphate metabolism in cancer and metabolic disease. Front Endocrinol (Lausanne). 2018; 9:177.
Burczynski M E, Oestreicher J L, Cahilly M J, et al. Clinical pharmacogenomics and transcriptional profiling in early phase oncology clinical trials. Curr Mol Med. 2005; 5:83-102.
Cardoso F, van′t Veer L J, Bogaerts J, et al. 70-gene signature as an aid to treatment decisions in early-stage breast cancer. N Engl J Med. 2016; 375:717-29.
Cejovic J, Radenkovic J, Mladenovic V, et al. Using semantic web technologies to enable cancer genomics discovery at petabyte scale. Cancer Inform. 2018 Sep. 28; 17: 1176935118774787.
Cooper L A, Demicco E G, Saltz J H, et al. PanCancer insights from The Cancer Genome Atlas: the pathologist's perspective. J Pathol. 2018; 244:512-524.
Dang L, Yen K, Attar E C. IDH mutations in cancer and progress toward development of targeted therapeutics. Ann Oncol. 2016; 27:599-608.
Dolezal J M, Dash A P, Prochownik E V. Diagnostic and prognostic implications of ribosomal protein transcript expression patterns in human cancers. BMC Cancer. 2018; 18:275.
Frye M, Harada B T, Behm M, et al. RNA modifications modulate gene expression during development. Science. 2018. 361; 1346-1349.
Galvani E, Peters G J, Giovannetti E. Thymidylate synthase inhibitors for non-small cell lung cancer. Expert Opin Investig Drugs. 2011; 20:1343-56.
Golub T R, Slonim D K, Tamayo P, et al. Molecular classification of cancer: class discovery and class prediction by gene expression monitoring. Science. 1999; 286:531-7.
Hanahan D, Weinberg R A. Hallmarks of cancer: the next generation. Cell. 2011.144; 646-74.
Ho T K. The random subspace method for constructing decision forests. IEEE Transactions on Pattern Analysis and Machine Intelligence. 1998: 20: 832-844.
Icard P, Lincet H. A global view of the biochemical pathways involved in the regulation of the metabolism of cancer cells. Biochim Biophys Acta. 2012; 1826:423-33.
Kalkat M, De Melo J, Hickman K A, et al. MYC Deregulation in Primary Human Cancers. Genes (Basel). 2017; 8. pii: E151.
Kim H, Park J, Wang J I, et al. Recent advances in proteomic profiling of pancreatic ductal adenocarcinoma and the road ahead. Expert Rev Proteomics, 2017; 14:963-971.
Knijnenburg T A, Wang L, Zimmermann M T, et al. Genomic and molecular landscape of DNA damage repair deficiency across the cancer genome atlas. Cell Rep. 2018; 23:239-254.
Kulkarni S, Dolezal J M, Wang H, et al. Ribosomopathy-like properties of murine and human cancers.
Levine A J, Puzio-Kuter A M. The control of the metabolic switch in cancers by oncogenes and tumor suppressor genes. Science. 2010 Dec. 3; 330(6009):1340-4.
Liu Q, Yu Z, Xiang Y, et al. Prognostic and predictive significance of thymidylate synthase protein expression in non-small cell lung cancer: a systematic review and meta-analysis. Cancer Biomark. 2015; 15:65-78.
Moreno-Sanchez R, Marin-Hernandez A, Saavedra E, et al. Who controls the ATP supply in cancer cells? Biochemistry lessons to understand cancer energy metabolism. Int J Biochem Cell Biol. 2014 May; 50:10-23.
Muller P A, Vousden K H. p53 mutations in cancer. Nat Cell Biol. 2013; 15:2-8.
Nesbit C E, Tersak J M, Prochownik E V. MYC oncogenes and human neoplastic disease. Oncogene. 1999 May 13; 18(19):3004-16.
Nikiforova M N. Mercurio S, Wald A I, et al. Analytical performance of the ThyroSeq v3 genomic classifier for cancer diagnosis in thyroid nodules. Cancer. 2018; 124:1682-1690.
Pelletier J, Thomas G, Volarević S. Ribosome biogenesis in cancer: new players and therapeutic avenues. Nat Rev Cancer. 2018; 18:51-63.
PLoS One. 2017; 12:e0182705.
Porter J R, Fisher B E, Batchelor E. p53 pulses diversify target gene expression dynamics in an mRNA half-life-dependent manner and delineate co-regulated target gene subnetworks. Cell Syst. 2016; 2:272-82.
Riganti C, Gazzano E, Polimeni M, et al. The pentose phosphate pathway: an anti-oxidant defense and a crossroad in tumor cell fate. Free Radic Biol Med. 2012 Aug. 1; 53(3):421-36.
Ross J. mRNA stability in mammalian cells. Microbiol Rev. 1995; 59:423-50.
Sanchez-Vega F, Mina M, Armenia J, et al. Oncogenic signaling pathways in the cancer genome atlas. Cell. 2018; 173:321-337.
Soutourina J. Transcription regulation by the Mediator complex. Nat Rev Mol Cell Biol. 2018; 19:262-274.
van de Vijver M J, He Y D, van't Veer L J, et al. A gene-expression signature as a predictor of survival in breast cancer. N Engl J Med. 2002; 347:1999-2009.
van der Maaten LJPH. Visualizing high-dimensional data using t-SNE. J Mach Learn Res. 2008; 9:2579-605.
Vogelstein B, Papadopoulos N, Velculescu V E, et al. Cancer genome landscapes. Science. 2013; 339:1546-58.
Wang H, Dolezal J M, Kulkami S, et al. Myc and ChREBP transcription factors cooperatively regulate normal and neoplastic hepatocyte proliferation in mice. J Biol Chem. 2018; 293:14740-14757.
Wong R W J, Ngoc PCT, Leong W Z, et al. Enhancer profiling identifies critical cancer genes and characterizes cell identity in adult T-cell leukemia. Blood. 2017; 130:2326-2338

1. A method for diagnosing, monitoring the progress of, and/or providing a prognosis of a cancer in a subject, said method comprising
a) receiving RNA expression data for a sample of tumor;
b) determining a global cancer pathway transcript (CPT) expression profile for the sample based on the RNA expression data for one or more cancer-related pathways; and
c) providing a diagnosis, prognosis, or treatment recommendation based on the global CPT expression profile;
wherein a change in one or more cancer pathway transcripts relative to a control indicates an increase in survivability of the subject for the cancer.

2. The method of claim 1, wherein the one or more cancer-related pathways is selected from the group consisting of Cell cycle, Notch, Purine biosynthesis, TP53, Hippo, TCA cycle, Wnt, PI3K, Pyrimidine Biosynthesis, TGF-β, Myc, and Pentose Phosphate Pathway (PPP).

3. The method of claim 2, wherein the one or more cancer-related pathways comprises cell cycle and the cancer pathway transcript comprises one or more of CDKN1A, CCND2, CDKN1B, CCND1, CDK4, CCND3, CDKN2C, CCNE1, CDK5, E2F3, CDK2, CDKN2A, RB1, E2F1, or CDKN2B.

4. The method of claim 2, wherein the one or more cancer-related pathways comprises the Wnt pathway and the cancer pathway transcript comprises one or more of ZNFR3, WIF1, TLE1, TLE2, TLE3, TLE4, TCF7L1, TCF7L2, SFRP1, SFRP2, SFRP4, SFRP5, RNF43, LRP5, GSK3B, DKK4, DKK3, DKK2, DKK1, CTNNB1, AXIN1, AXIN2, APC, or AMER1.

5. The method of claim 2, wherein the one or more cancer-related pathways comprises the TP53 pathway and the cancer pathway transcript comprises one or more of TP53, CHEK2, MDM4, RPS6KA3, MDM2, or ATM.

6. The method of claim 2, wherein the one or more cancer-related pathways comprises the TGF-β pathway and the cancer pathway transcript comprises one or more of TGFBR2, TGFBR1, ACVR1B, ACVR2A, SMAD2, SMAD3, or SMAD4.

7. The method of claim 2, wherein the one or more cancer-related pathways comprises the Notch pathway and the cancer pathway transcript comprises one or more of NOV, DNER, HDAC1, HES1, HES2, HES5, HES4, HES5, HEY1, CREBBP, CNTN6, NOTCH2, NOTCH1, NCOR1, FBXW7, HEYL, NOTCH4, NCOR2, NES2, NOTCH3, PSEN2, KDM5A, EP300, KAT2B, SPEN, JAG2, HEY2, THBS2, CUL1, MAML3, or ARRDC1.

8. The method of claim 2, wherein the one or more cancer-related pathways comprises the PI3K pathway and the cancer pathway transcript comprises one or more of PTEN, PIK3CB, AKT3, PPP2R1A, PIK3R1, RICTOR, RHEB, TSC2, PIK3CA, MTOR, AKT2, STK11, AKT1, TSC1, RPTOR, PIK3R2, INPP4B, or PIK3R3.

9. The method of claim 2, wherein the one or more cancer-related pathways comprises the Hippo pathway and the cancer pathway transcript comprises one or more of YAP1, WWTR1, TEAD2, STK4, STK3, SAV1, LATS1, LATS2, MOB1A, MOB1B, PTPN14, NF2, WWC1, TAOK1, TAOK2, TAOK3, CRB1, CRB2, CRB3, FAT1, FAT2, FAT3, FAT4, DCHS1, DCHS2, CSNK1E, or CSNK1D.

10. The method of claim 2, wherein the one or more cancer-related pathways comprises the Myc pathway and the cancer pathway transcript comprises one or more of MXD4, MLXIPL, MAX, MXI1, MYC, N-MYC, MXD1, MXD2, MXD3, MLX, MNT, MYCL, MLXIP, MYCN, or MGA.

11. The method of claim 2, wherein the one or more cancer-related pathways comprises the purine biosynthesis pathway and the cancer pathway transcript comprises one or more of PPAT, GART, PFAS, PAICS, ADSL, ATIC, ADSSL1, ADSS, AK1, AK2, AK3, AK4, AK5, AK7, GMPS, GUK1, RRM1, RRM2, NME1, NME2, NME3, NME4, NME5, NME6, or NME7.

12. The method of claim 2, wherein the one or more cancer-related pathways comprises the pyrimidine biosynthesis pathway and the cancer pathway transcript comprises one or more of NME4, NME3, RRM1, CMPK1, NME5, CAD, DUT, ENPP3, CMPK2, NTPCR, RRM2, CTPS1, NME6, NME2, DHODH, ITPA, TYMS, NME7, NME1, UMPS, DTYMK, ENPP1, or CPTS2.

13. The method of claim 2, wherein the one or more cancer-related pathways comprises the TCA pathway and the cancer pathway transcript comprises one or more of CS, IDH1, IDH2, SDHD, OGDH, IDH3A, SUCLA2, IDH3B, SDHA, OGDHL, SUCLG1, FH, ACO2, SUCLG2, MDH1, SDHB, ACO1, MDH1B, IDH3G, MDH2, or SDHC.

14. The method of claim 2, wherein the one or more cancer-related pathways comprises the PPP pathway and the cancer pathway transcript comprises one or more of PGD, H6PD, TALDO1, PGLS, TKT, RPIA, RPE, G6PD, TKTL1, TKTL2, or RPEL1.

15. The method of claim 1, wherein the cancer is selected from the group consisting of Acute myeloid leukemia (AML), Adrenocortical carcinoma (ACC), Bladder urothelial carcinoma (BLCA), Brain lower grade Glioma (BLGG), Breast invasive carcinoma (BRIC), triple negative breast cancer (TNBC), luminal A breast cancer, cervical squamous cell carcinoma and endocervical adenocarcinoma (CESC), Cholangiocarcinoma (CHOL), Glioblastoma multiform (GBM), Head and neck squamous cell carcinoma (HNSC), High risk Wilms tumor (HRWT), Kidney chromophobe (KICH), Clear cell renal cancer (KIRC), Kidney renal papillary cell carcinoma (KURP), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromacytoma/paraganglioneuroma (PCPG), Rectal adeno-carcinoma (READ), Sarcoma (SARC), Metastatic skin cutaneous melanoma (Metastatic SKCM), Stomach adenocarcinoma (STAD), Thymoma (THYM), Thyroid cancer (THYC), Uterine carcinosarcoma (UCSC), Uterine corpus endometrial carcinoma (UCEC), and Uveal melanoma (UVM).

16. The method of claim 15, wherein the cancer is not colon adenocarcinoma (COAD), esophageal cancer (ESOP), diffuse large B-cell lymphoma (DLBC), prostate cancer (PRAD), or testicular germ cell tumor (TGCT).

17. The method of claim 1, wherein the cancer comprises AML and the cancer related pathways comprise one or more of cell cycle, PI3K, Hippo, Purine Biosynthesis, and TCA; wherein the cancer comprises ACC and the cancer related pathways comprise one or more of cell cycle, TP53, TGF-β, Notch, Myc, Pyrimidine Biosynthesis, and TCA; wherein the cancer comprises BLCA and the cancer related pathways comprise one or more of TGF-β, Notch, Myc, Purine Biosynthesis, and TCA; wherein the cancer comprises BLGG and the cancer related pathways comprise one or more of cell cycle, TP53, TGF-β, PI3K, Hippo, Myc, Purine biosynthesis, and PPP; wherein the cancer related pathways comprise one or more of PI3K, Myc, Purine biosynthesis, and Hippo; wherein the cancer comprises BRIC and the cancer related pathways comprise one or more of cell cycle, TP53, Myc, Purine Biosynthesis, and Pyrimidine Biosynthesis; wherein the cancer comprises CESC and the cancer related pathways comprise one or more of cell cycle, Myc, and Purine Biosynthesis; wherein the cancer comprises CHOL and the cancer related pathways comprise one or more of Notch and Myc; wherein the cancer comprises GBM and the cancer related pathways comprises TP53; wherein the cancer comprises HNSC and the cancer related pathways comprise one or more of cell cycle, and Myc; wherein the cancer comprises HRWT and the cancer related pathways comprise one or more of Wnt, TGF-β, Notch, PI3K, and Myc; wherein the cancer comprises KICH and the cancer related pathways comprise one or more of cell cycle, Wnt, PI3K, Purine Biosynthesis, and Pyrimidine Biosynthesis; wherein the cancer comprises KIRC and the cancer related pathways comprise one or more of cell cycle, Wnt, TP53, TGF-β, Hippo, Myc, Purine Biosynthesis, and TCA; wherein the cancer comprises KIRC and the cancer related pathways comprise one or more of Wnt, Pyrimidine Biosynthesis, Myc, and TCA; wherein the cancer comprises KURP and the cancer related pathways comprise one or more of cell cycle, PI3K, Hippo, Purine Biosynthesis, Pyrimidine Biosynthesis, TCA, and PPP; wherein the cancer comprises LIHC and the cancer related pathways comprise one or more of Wnt, Purine Biosynthesis, TCA, and PPP; wherein the cancer comprises LUAD and the cancer related pathways comprise one or more of Wnt, PI3K, and Myc; wherein the cancer comprises LUSC and the cancer related pathways comprise one or more of cell cycle, Wnt, Hippo, and Purine Biosynthesis; wherein the cancer comprises MESO and the cancer related pathways comprise one or more of cell cycle, TGF-β, Notch, PI3K, Hippo, Purine Biosynthesis, Pyrimidine biosynthesis, and PPP; wherein the cancer comprises OV and the cancer related pathways comprises cell cycle; wherein the cancer comprises PAAD and the cancer related pathways comprise one or more of cell cycle, Myc, and Purine Biosynthesis; wherein the cancer comprises PCPG and the cancer related pathways comprises Wnt; wherein the cancer comprises READ and the cancer related pathways comprises cell cycle; wherein the cancer comprises SARC and the cancer related pathways comprise one or more of TGF-β, Myc, Purine Biosynthesis, Pyrimidine biosynthesis, and PPP; wherein the cancer comprises metastatic SKCM and the cancer related pathways comprise one or more of Wnt, Notch, and Hippo; wherein the cancer comprises STAD and the cancer related pathways comprise one or more of TGF-β and Hippo; wherein the cancer comprises THYM and the cancer related pathways comprise one or more of cell cycle, Wnt, TP53, Hippo, Purine Biosynthesis, Pyrimidine biosynthesis, and PPP; wherein the cancer comprises THYC and the cancer related pathways comprise one or more of cell cycle, PI3K, and TCA; wherein the cancer comprises UCSC and the cancer related pathways comprises TP53; wherein the cancer comprises UCEC and the cancer related pathways comprise one or more of cell cycle, Wnt, Notch, Purine Biosynthesis, and Pyrimidine biosynthesis; wherein the cancer comprises UVM and the cancer related pathways comprise one or more of cell cycle, Wnt, TCA, and PPP; wherein the cancer comprises breast cancer and the cancer related pathways comprise one or more of Wnt and Myc; wherein the cancer comprises TNBC and the cancer related pathways comprise one or more of Wnt and Myc; or wherein the cancer comprises luminal A breast cancer and the cancer related pathways comprise one or more of Myc.

18-50. (canceled)

51. The method of claim 1, further comprising:
receiving the sample of tumor;
extracting RNA from the sample;
isolating a plurality of CPTs from the extracted RNA; and
obtaining the RNA expression data from the isolated CPTs.

52. (canceled)

53. (canceled)

54. The method of claim 1, further comprising:
a) receiving respective RNA expression data and respective clinical information for each of a plurality of tumors from a database;
b) determining respective global CPT expression profiles for the tumors in the database based on the respective RNA expression data;
c) identifying recurring patterns of CPT expression among the tumors in the database; and
d) comparing the recurring patterns of CPT expression with the respective clinical parameters.

55. The method of claim 54, wherein identifying recurring patterns of CPT expression among tumors in the database further comprises applying a machine learning model that analyzes linear and non-linear relationships among the respective relative expression for each of the plurality of CPTs.

56. (canceled)
